SEQUENCE LISTING (60 sequences)

SEQ ID NO: 1 - protein, 115 aa, homo sapiens
IPTEIPTSALVKETLALLSTHRTLLIANETLRIPVPVHKNHQLCTEEIFQGIGTLESQTVQGGTVERLFKNLSLIKKYIDGQKKKCGEERRRVNQFLDYLQEFLGVMNTEWIIES

SEQ ID NO: 2 - DNA, 45 bp, Artificial sequence
  Tetanus toxoid P2 epitope
cagtacatcaaagctaactccaaattcatcggcatcaccgaactg

SEQ ID NO: 3 - protein, 15 aa, Artificial sequence
  Tetanus toxoid P2 epitope
QYIKANSKFIGITEL

SEQ ID NO: 4 - DNA, 63 bp, Artificial sequence
  Tetanus toxoid P30 epitope
ttcaacaacttcaccgtttccttctggctgcgcgttccaaaagtttccgcttcccacctggaa

SEQ ID NO: 5 - protein, 21 aa, Artificial sequence
  Tetanus toxoid P30 epitope
FNNFTVSFWLRVPKVSASHLE

SEQ ID NO: 6 - DNA, 39 bp, Artificial sequence
  Pan DR binding peptide (PADRE)
gccaagttcgtggccgcttggaccctgaaggccgcagct

SEQ ID NO: 7 - protein, 13 aa, Artificial sequence
  Pan DR binding peptide (PADRE)
AKFVAAWTLKAAA

SEQ ID NO: 8 - DNA, 858 bp, Artificial sequence
  2 human IL5 monomers joined by P30 and P2 epitopes
atgaggatgcttctgcatttgagtttgctggctcttggagctgcctacgtgtatgccatccccacagaaattcccacaagtgcattggtgaaagagaccttggcactgctttctactcatcgaactctgctgatagccaatgagactctgaggattcctgttcctgtacataaaaatcaccaactgtgcactgaagaaatctttcagggaataggcacactggagagtcaaactgtgcaagggggtactgtggaaagactattcaaaaacttgtccttaataaagaaatacattgacggccaaaaaaaaaagtgtggagaagaaagacggagagtaaaccaattcctagactacctgcaagagtttcttggtgtaatgaacaccgagtggataatagaaagtttcaacaacttcaccgtgagcttctggctgcgcgtgcctaaggtgagcgccagccacctggagcagtacatcaaggccaactccaagttcatcggcatcaccgagctgatccccacagaaattcccacaagtgcattggtgaaagagaccttggcactgctttctactcatcgaactctgctgatagccaatgagactctgaggattcctgttcctgtacataaaaatcaccaactgtgcactgaagaaatctttcagggaataggcacactggagagtcaaactgtgcaagggggtactgtggaaagactattcaaaaacttgtccttaataaagaaatacattgacggccaaaaaaaaaagtgtggagaagaaagacggagagtaaaccaattcctagactacctgcaagagtttcttggtgtaatgaacaccgagtggataatagaaagttga

SEQ ID NO: 9 - protein, 285 aa, Artificial sequence
  2 human IL5 monomers joined by P30 and P2 epitopes
MRMLLHLSLLALGAAYVYAIPTEIPTSALVKETLALLSTHRTLLIANETLRIPVPVHKNHQLCTEEIFQGIGTLESQTVQGGTVERLFKNLSLIKKYIDGQKKKCGEERRRVNQFLDYLQEFLGVMNTEWIIESFNNFTVSFWLRVPKVSASHLEQYIKANSKFIGITELIPTEIPTSALVKETLALLSTHRTLLIANETLRIPVPVHKNHQLCTEEIFQGIGTLESQTVQGGTVERLFKNLSLIKKYIDGQKKKCGEERRRVNQFLDYLQEFLGVMNTEWIIES

SEQ ID NO: 10 - DNA, 858 bp, Artificial sequence
  2 human IL5 monomers joined by P2 and P30 epitopes
atgaggatgcttctgcatttgagtttgctggctcttggagctgcctacgtgtatgccatccccacagaaattcccacaagtgcattggtgaaagagaccttggcactgctttctactcatcgaactctgctgatagccaatgagactctgaggattcctgttcctgtacataaaaatcaccaactgtgcactgaagaaatctttcagggaataggcacactggagagtcaaactgtgcaagggggtactgtggaaagactattcaaaaacttgtccttaataaagaaatacattgacggccaaaaaaaaaagtgtggagaagaaagacggagagtaaaccaattcctagactacctgcaagagtttcttggtgtaatgaacaccgagtggataatagaaagtcagtacatcaaggccaactccaagttcatcggcatcaccgagctgttcaacaacttcaccgtgagcttctggctgcgcgtgcctaaggtgagcgccagccacctggagatccccacagaaattcccacaagtgcattggtgaaagagaccttggcactgctttctactcatcgaactctgctgatagccaatgagactctgaggattcctgttcctgtacataaaaatcaccaactgtgcactgaagaaatctttcagggaataggcacactggagagtcaaactgtgcaagggggtactgtggaaagactattcaaaaacttgtccttaataaagaaatacattgacggccaaaaaaaaaagtgtggagaagaaagacggagagtaaaccaattcctagactacctgcaagagtttcttggtgtaatgaacaccgagtggataatagaaagttga

SEQ ID NO: 11 - protein, 285 aa, Artificial sequence [sequence omitted]

SEQ ID NO: 12 - DNA, 864 bp, Artificial sequence
  Two human IL5 monomers joined by diglycine linker and including terminally positioned P30 and P2 epitopes
atgaggatgcttctgcatttgagtttgctggctcttggagctgcctacgtgtatgccatccccacagaattcaacaacttcaccgtgagcttctggctgcgcgtgcctaaggtgagcgccagccacctggagattcccacaagtgcattggtgaaagagaccttggcactgctttctactcatcgaactctgctgatagccaatgagactctgaggattcctgttcctgtacataaaaatcaccaactgtgcactgaagaaatctttcagggaataggcacactggagagtcaaactgtgcaagggggtactgtggaaagactattcaaaaacttgtccttaataaagaaatacattgacggccaaaaaaaaaagtgtggagaagaaagacggagagtaaaccaattcctagactacctgcaagagtttcttggtgtaatgaacaccgagtggataatagaaagtggcggtatccccacagaaattcccacaagtgcattggtgaaagagaccttggcactgctttctactcatcgaactctgctgatagccaatgagactctgaggattcctgttcctgtacataaaaatcaccaactgtgcactgaagaaatctttcagggaataggcacactggagagtcaaactgtgcaagggggtactgtggaaagactattcaaaaacttgtccttaataaagaaatacattgacggccaaaaaaaaaagtgtggagaagaaagacggagagtaaaccaattcctagactacctgcaagagtttcttggtgtaatgaacaccgagtggataatagaaagtcagtacatcaaggccaactccaagttcatcggcatcaccgagctgtga

SEQ ID NO: 13 - protein, 287 aa, Artificial sequence [sequence omitted]

SEQ ID NO: 14 - DNA, 864 bp, Artificial sequence
  Two human IL5 monomers joined by a di-glycine linker and including terminally positioned tetanus toxoid P2 and P30 epitopes
atgaggatgcttctgcatttgagtttgctggctcttggagctgcctacgtgtatgccatccccacagaacagtacatcaaggccaactccaagttcatcggcatcaccgagctgattcccacaagtgcattggtgaaagagaccttggcactgctttctactcatcgaactctgctgatagccaatgagactctgaggattcctgttcctgtacataaaaatcaccaactgtgcactgaagaaatctttcagggaataggcacactggagagtcaaactgtgcaagggggtactgtggaaagactattcaaaaacttgtccttaataaagaaatacattgacggccaaaaaaaaaagtgtggagaagaaagacggagagtaaaccaattcctagactacctgcaagagtttcttggtgtaatgaacaccgagtggataatagaaagtggcggtatccccacagaaattcccacaagtgcattggtgaaagagaccttggcactgctttctactcatcgaactctgctgatagccaatgagactctgaggattcctgttcctgtacataaaaatcaccaactgtgcactgaagaaatctttcagggaataggcacactggagagtcaaactgtgcaagggggtactgtggaaagactattcaaaaacttgtccttaataaagaaatacattgacggccaaaaaaaaaagtgtggagaagaaagacggagagtaaaccaattcctagactacctgcaagagtttcttggtgtaatgaacaccgagtggataatagaaagtttcaacaacttcaccgtgagcttctggctgcgcgtgcctaaggtgagcgccagccacctggagtga

SEQ ID NO: 15 - protein, 287 aa, Artificial sequence [sequence omitted]

SEQ ID NO: 16 - DNA, 477 bp, Artificial sequence
  Human wt TNF (codons optimised)
atggtgcgctcaagctcgcgcacgccgagtgacaaaccagtagctcatgttgtggccaaccctcaggcggaaggccagctccaatggttaaatcgtcgcgcgaacgccctgctggcgaacggcgtggaactgcgtgataaccagctggtggtccccagcgaggggctgtatctgatctattcacaggtgttgtttaagggtcagggttgtccgagcacccacgttctgctgacgcataccatttctcgtattgctgtatcttatcaaactaaagtcaatttactttcggcgatcaaatccccgtgccaacgtgagacccctgaaggagcggaagccaaaccttggtacgaaccgatctatctggggggcgtttttcagctcgaaaaaggtgatcggctgagcgccgaaattaatcgcccggactaccttgatttcgcagagtccggtcaggtctacttcggcattatcgcattgtaa

SEQ ID NO: 17 - protein, 158 aa, Artificial sequence
  Human wt TNF (codons optimised)
MVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 18 - protein, 170 aa, Artificial sequence
  hTNF with inserted PADRE
VRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAKFVAAWTLKAAAAEAKPWYEPIYLGGVFQLEKGDRLSAEINRPDYLDFAESGQVYFGIIAL

SEQ ID NO: 19 - DNA, 39 bp, Artificial sequence
  Pan DR binding peptide (PADRE)
gcgaagttcgttgcagcttggaccctgaaggccgctgca

SEQ ID NO: 20 - protein, 13 aa, Artificial sequence
  Pan DR binding peptide (PADRE)
AKFVAAWTLKAAA

SEQ ID NO: 21 - DNA, 1437 bp, Artificial sequence
  Monomeric mimic of trimeric human TNF
atggtgcgcagcagcagccgcacccccagcgacaagcccgtggcccacgtggtggccaacccccaggccgagggccaactgcagtggctgaaccgccgcgccaacgccctgctggcaaacggcgtggagctgcgcgacaaccagctggtggtgcccagcgagggcctgtacctgatctacagccaggtgctgttcaagggccagggctgccccagcacccacgtgctgctgacccacaccatcagccgcatcgccgtgagctaccagaccaaggtgaacctgctgagcgccatcaagagcccctgccagcgcgagacccccgagggcgccgaggccaagccctggtacgagcccatctacctcggcggcgtgttccagctggagaagggcgaccgcctgagcgccgagatcaaccgccccgactacctggacttcgccgagagcggccaggtgtacttcggcatcatcgccctgggtggcggagtccggtcctcctcccggacaccatccgacaaaccagtcgctcatgtcgtcgctaatccacaagctgaaggtcaacttcaatggcttaatcggcgggctaatgctcttcttgctaatggtgtcgaacttcgggacaatcaacttgtcgtcccatccgaaggtctttatcttatttattcccaagtcctttttaaaggtcaaggttgtccatccacacatgtccttcttacacatacaatttcccggattgctgtctcctatcaaacaaaagtcaatcttctttccgctattaaatccccatgtcaacgggaaacaccagaaggtgctgaagctaaaccttggtatgaaccaatttatcttggtggtgtctttcaacttgaaaaaggtgaccggctttccgctgaaattaatcggccagattatcttgactttgctgaatccggtcaagtctattttggtattattgctctgggcggtggggttcgttcttcttctcgtacgccgtctgataagccggttgcgcacgttgttgcgaacccgcaggcggaggggcaattgcagtggttgaatcgtcgtgcgaacgcgttgttggcgaatggggttgaattgcgtgataaccaattggttgttccgtctgaggggttgtacttgatatattctcaggttttgttcaaagggcaagggtgcccgtctacgcatgttttgttgacgcacacgatatctcgtatagcggtttcttaccagacgaaggttaatttgttgtctgcgataaaatctccgtgtcaacgtgaaacgccggaaggggcggaggcgaagccgtggtatgaaccgatatacttggggggggtttttcagttggaaaaaggggatcgtttgtctgcggagataaaccgtccggactatttggatttcgcggaatctgggcaagtttactttgggataatagcgctgtaa

SEQ ID NO: 22 - protein, 478 aa, Artificial sequence
  Monomeric mimic of trimeric human TNF
MVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGGVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGGVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 23 - protein, 170 aa, Artificial sequence
  hTNF with inserted PADRE
VRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPAKFVAAWTLKAAAEGAEAKPWYEPIYLGGVFQLEKGDRLSAEINRPDYLDFAESGQVYFGIIAL

SEQ ID NO: 24 - protein, 170 aa, Artificial sequence
  hTNF with inserted PADRE
VRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEAKFVAAWTLKAAAGAEAKPWYEPIYLGGVFQLEKGDRLSAEINRPDYLDFAESGQVYFGIIAL

SEQ ID NO: 25 - protein, 169 aa, Artificial sequence
  hTNF with 12 amino acids of PADRE inserted
VRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAKFVAAWTLKAAAEAKPWYEPIYLGGVFQLEKGDRLSAEINRPDYLDFAESGQVYFGIIAL

SEQ ID NO: 26 - protein, 167 aa, Artificial sequence
  hTNF with PADRE substituted in
VRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAKFVAAWTLKAAAKPWYEPIYLGGVFQLEKGDRLSAEINRPDYLDFAESGQVYFGIIAL

SEQ ID NO: 27 - protein, 165 aa, Artificial sequence
  hTNF with in-substituted PADRE
VRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPAKFVAAWTLKAAAKPWYEPIYLGGVFQLEKGDRLSAEINRPDYLDFAESGQVYFGIIAL

SEQ ID NO: 28 - protein, 173 aa, Artificial sequence
  hTNF with C-terminal tri-glycine linker and PADRE
VRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGGAKFVAAWTLKAAA

SEQ ID NO: 29 - protein, 170 aa, Artificial sequence
  hTNF with inserted PADRE and additional disulfide bridge
VRSSSRTPSDKPVAHVVANPQAEGQLQWLNRRANALLANGVELRDNQLVVPSEGLYLIYSQVLFKGCGCPSTHVLLTHTISRIAVSYQTKVNLLSAIKSPCQRETPEGAKFVAAWTLKAAAAECKPWYEPIYLGGVFQLEKGDRLSAEINRPDYLDFAESGQVYFGIIAL

SEQ ID NO: 30 - protein, 170 aa, Artificial sequence
  hTNF with inserted PADRE and additional disulphide bridge
VRSSSRTPSDKPVAHVVANPQAEGQLQWLNRRANALLANGVELRDNQLVVPSEGLYLIYSQVLFKGQGCPSTHVLLTHTISRIAVSYQTKVNLLSCIKSPCQRETPEGAKFVAAWTLKAAAAEAKPWYEPCYLGGVFQLEKGDRLSAEINRPDYLDFAESGQVYFGIIAL

SEQ ID NO: 31 - protein, 162 aa, Artificial sequence
  hTNF truncate with inserted PADRE
SDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAKFVAAWTLKAAAAEAKPWYEPIYLGGVFQLEKGDRLSAEINRPDYLDFAESGQVYFGIIAL

SEQ ID NO: 32 - protein, 169 aa, Artificial sequence
  hTNF with PADRE inserted
VRSSSRTPSDKPVAHVVAKFVAAWTLKAAANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 33 - protein, 165 aa, Artificial sequence
  hTNF with in-substituted PADRE
VRSSSRTPSDKPVAHVVAKFVAAWTLKAAAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 34 - protein, 170 aa, Artificial sequence
  hTNF with added artificial stalk region and inserted PADRE
MAKFVAAWTLKAAARSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 35 - protein, 170 aa, Artificial sequence
  hTNF with inserted PADRE and single stabilising mutation
VRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAKFVAAWTLKAAAAEAKPWYEPIYLGGVFQLEKGDRLSAEINRPDYLDFAESGQVYFGIIAF

SEQ ID NO: 36 - protein, 170 aa, Artificial sequence
  hTNF with inserted PADRE and one single mutation
VRSSSRTPSDKPVAHVVANPQAEGQLQWLNRRANALLANGVELRDNQLFVPSEGLYLIYSQVLFKGQGCPSTHVLLTHTISRIAVSYQTKVNLLSAIKSPCQRETPEGAKFVAAWTLKAAAAEAKPWYEPIYLGGVFQLEKGDRLSAEINRPDYLDFAESGQVYFGIIAL

SEQ ID NO: 37 - protein, 174 aa, Artificial sequence
  hTNF with inserted glycine-linked PADRE
VRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGGGAKFVAAWTLKAAAGGAEAKPWYEPIYLGGVFQLEKGDRLSAEINRPDYLDFAESGQVYFGIIAL

SEQ ID NO: 38 - protein, 167 aa, Artificial sequence
  hTNF with in-substituted PADRE
VRSSSRTPSDKPVAHVVANPQAEGQLQWLNRRANALLANGVELRDNQLVVPSEGLYLIYSQVLFKGQGCPSTHVLLTHTISRIAKFVAAWTLKAAAYQTKVNLLSAIKSPCQRETPEGAEAKPWYEPIYLGGVFQLEKGDRLSAEINRPDYLDFAESGQVYFGIIAL

SEQ ID NO: 39 - protein, 157 aa, Artificial sequence
  hTNF with in-substituted PADRE
VRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLAKFVAAWTLKAAAESGQVYFGIIAL

SEQ ID NO: 40 - protein, 160 aa, Artificial sequence
  hTNF with in-substituted PADRE
VRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEAKFVAAWTLKAAAESGQVYFGIIAL

SEQ ID NO: 41 - protein, 157 aa, Artificial sequence
  hTNF with insubstituted PADRE
VRSSSRTPSDKPVAHVVANPQAEGQLQWLNRRANALLANGVELRDNQLVVPSEGLYLIYSQVLAKFVAAWTLKAAATHTISRIAVSYQTKVNLLSAIKSPCQRETPEGAEAKPWYEPIYLGGVFQLEKGDRLSAEINRPDYLDFAESGQVYFGIIAL

SEQ ID NO: 42 - protein, 157 aa, Artificial sequence
  hTNF with in-substituted PADRE
VRSSSRTPSDKPVAHVVANPQAEGQLQWLNRRANALLANGVELRDNQLVVPSEGLYLIYSQVLFKGQGCPSAKFVAAWTLKAAAVSYQTKVNLLSAIKSPCQRETPEGAEAKPWYEPIYLGGVFQLEKGDRLSAEINRPDYLDFAESGQVYFGIIAL

SEQ ID NO: 43 - protein, 157 aa, Artificial sequence
  hTNF with insubstituted PADRE
VRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLAKFVAAWTLKAAADYLDFAESGQVYFGIIAL

SEQ ID NO: 44 - protein, 176 aa, Artificial sequence
  hTNF with inserted peptide and duplication of 6 amino acids
VRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAKFVAAWTLKAAARETPEGAEAKPWYEPIYLGGVFQLEKGDRLSAEINRPDYLDFAESGQVYFGIIAL

SEQ ID NO: 45 - protein, 174 aa, Artificial sequence
  hTNF with inserted PADRE and duplication of 4 amino acids
VRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAKFVAAWTLKAAATPEGAEAKPWYEPIYLGGVFQLEKGDRLSAEINRPDYLDFAESGQVYFGIIAL

SEQ ID NO: 46 - protein, 194 aa, Artificial sequence
  hTNF with inserted tetanus toxoid P2 and P30 epitopes
MVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGQYIKANSKFIGITELFNNFTVSFWLRVPKVSASHLEAEAKPWYEPIYLGGVFQLEKGDRLSAEINRPDYLDFAESGQVYFGIIAL

SEQ ID NO: 47 - protein, 194 aa, Artificial sequence
  hTNF with inserted tetanus toxoid P2 and P30 epitopes
MVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGFNNFTVSFWLRVPKVSASHLEQYIKANSKFIGITELAEAKPWYEPIYLGGVFQLEKGDRLSAEINRPDYLDFAESGQVYFGIIAL

SEQ ID NO: 48 - DNA, 1545 bp, Artificial sequence
  3 hTNF sequences joined by glycince linkers and tetanus toxoid P2 and P30 epitopes
atggtgcgcagcagcagccgcacccccagcgacaagcccgtggcccacgtggtggccaacccccaggccgagggccaactgcagtggctgaaccgccgcgccaacgccctgctggcaaacggcgtggagctgcgcgacaaccagctggtggtgcccagcgagggcctgtacctgatctacagccaggtgctgttcaagggccagggctgccccagcacccacgtgctgctgacccacaccatcagccgcatcgccgtgagctaccagaccaaggtgaacctgctgagcgccatcaagagcccctgccagcgcgagacccccgagggcgccgaggccaagccctggtacgagcccatctacctcggcggcgtgttccagctggagaagggcgaccgcctgagcgccgagatcaaccgccccgactacctggacttcgccgagagcggccaggtgtacttcggcatcatcgccctgggtggcggacagtacatcaaagctaactccaaattcatcggcatcaccgaactggtccggtcctcctcccggacaccatccgacaaaccagtcgctcatgtcgtcgctaatccacaagctgaaggtcaacttcaatggcttaatcggcgggctaatgctcttcttgctaatggtgtcgaacttcgggacaatcaacttgtcgtcccatccgaaggtctttatcttatttattcccaagtcctttttaaaggtcaaggttgtccatccacacatgtccttcttacacatacaatttcccggattgctgtctcctatcaaacaaaagtcaatcttctttccgctattaaatccccatgtcaacgggaaacaccagaaggtgctgaagctaaaccttggtatgaaccaatttatcttggtggtgtctttcaacttgaaaaaggtgaccggctttccgctgaaattaatcggccagattatcttgactttgctgaatccggtcaagtctattttggtattattgctctgggcggtgggttcaacaacttcaccgtttccttctggctgcgcgttccaaaagtttccgcttcccacctggaagttcgttcttcttctcgtacgccgtctgataagccggttgcgcacgttgttgcgaacccgcaggcggaggggcaattgcagtggttgaatcgtcgtgcgaacgcgttgttggcgaatggggttgaattgcgtgataaccaattggttgttccgtctgaggggttgtacttgatatattctcaggttttgttcaaagggcaagggtgcccgtctacgcatgttttgttgacgcacacgatatctcgtatagcggtttcttaccagacgaaggttaatttgttgtctgcgataaaatctccgtgtcaacgtgaaacgccggaaggggcggaggcgaagccgtggtatgaaccgatatacttggggggggtttttcagttggaaaaaggggatcgtttgtctgcggagataaaccgtccggactatttggatttcgcggaatctgggcaagtttactttgggataatagcgctgtaa

SEQ ID NO: 49 - protein, 514 aa, Artificial sequence
  3 hTNF sequences joined by glycince linkers and tetanus toxoid P2 and P30 epitopes
MVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGGQYIKANSKFIGITELVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGGFNNFTVSFWLRVPKVSASHLEVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 50 - DNA, 1545 bp, Artificial sequence
  3 hTNF monomers joined by tri-glycine linkers and tetanus toxoid P2 and P30 epitopes
atggtgcgcagcagcagccgcacccccagcgacaagcccgtggcccacgtggtggccaacccccaggccgagggccaactgcagtggctgaaccgccgcgccaacgccctgctggcaaacggcgtggagctgcgcgacaaccagctggtggtgcccagcgagggcctgtacctgatctacagccaggtgctgttcaagggccagggctgccccagcacccacgtgctgctgacccacaccatcagccgcatcgccgtgagctaccagaccaaggtgaacctgctgagcgccatcaagagcccctgccagcgcgagacccccgagggcgccgaggccaagccctggtacgagcccatctacctcggcggcgtgttccagctggagaagggcgaccgcctgagcgccgagatcaaccgccccgactacctggacttcgccgagagcggccaggtgtacttcggcatcatcgccctgggtggcggattcaacaacttcaccgtttccttctggctgcgcgttccaaaagtttccgcttcccacctggaagtccggtcctcctcccggacaccatccgacaaaccagtcgctcatgtcgtcgctaatccacaagctgaaggtcaacttcaatggcttaatcggcgggctaatgctcttcttgctaatggtgtcgaacttcgggacaatcaacttgtcgtcccatccgaaggtctttatcttatttattcccaagtcctttttaaaggtcaaggttgtccatccacacatgtccttcttacacatacaatttcccggattgctgtctcctatcaaacaaaagtcaatcttctttccgctattaaatccccatgtcaacgggaaacaccagaaggtgctgaagctaaaccttggtatgaaccaatttatcttggtggtgtctttcaacttgaaaaaggtgaccggctttccgctgaaattaatcggccagattatcttgactttgctgaatccggtcaagtctattttggtattattgctctgggcggtgggcagtacatcaaagctaactccaaattcatcggcatcaccgaactggttcgttcttcttctcgtacgccgtctgataagccggttgcgcacgttgttgcgaacccgcaggcggaggggcaattgcagtggttgaatcgtcgtgcgaacgcgttgttggcgaatggggttgaattgcgtgataaccaattggttgttccgtctgaggggttgtacttgatatattctcaggttttgttcaaagggcaagggtgcccgtctacgcatgttttgttgacgcacacgatatctcgtatagcggtttcttaccagacgaaggttaatttgttgtctgcgataaaatctccgtgtcaacgtgaaacgccggaaggggcggaggcgaagccgtggtatgaaccgatatacttggggggggtttttcagttggaaaaaggggatcgtttgtctgcggagataaaccgtccggactatttggatttcgcggaatctgggcaagtttactttgggataatagcgctgtaa

SEQ ID NO: 51 - protein, 514 aa, Artificial sequence
  3 hTNF monomers joined by tri-glycine linkers and tetanus toxoid P2 and P30 epitopes
MVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGGFNNFTVSFWLRVPKVSASHLEVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGGQYIKANSKFIGITELVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 52 - DNA, 1554 bp, Artificial sequence
  3 hTNF monomers joined by tri-glycine linkers and with P2 and P30 epitopes introduced
atggtgcgcagcagcagccgcacccccagcgacaagcccgtggcccacgtggtggccaacccccaggccgagggccaactgcagtggctgaaccgccgcgccaacgccctgctggcaaacggcgtggagctgcgcgacaaccagctggtggtgcccagcgagggcctgtacctgatctacagccaggtgctgttcaagggccagggctgccccagcacccacgtgctgctgacccacaccatcagccgcatcgccgtgagctaccagaccaaggtgaacctgctgagcgccatcaagagcccctgccagcgcgagacccccgagggcgccgaggccaagccctggtacgagcccatctacctcggcggcgtgttccagctggagaagggcgaccgcctgagcgccgagatcaaccgccccgactacctggacttcgccgagagcggccaggtgtacttcggcatcatcgccctgggtggcggagtccggtcctcctcccggacaccatccgacaaaccagtcgctcatgtcgtcgctaatccacaagctgaaggtcaacttcaatggcttaatcggcgggctaatgctcttcttgctaatggtgtcgaacttcgggacaatcaacttgtcgtcccatccgaaggtctttatcttatttattcccaagtcctttttaaaggtcaaggttgtccatccacacatgtccttcttacacatacaatttcccggattgctgtctcctatcaaacaaaagtcaatcttctttccgctattaaatccccatgtcaacgggaaacaccagaaggtgctgaagctaaaccttggtatgaaccaatttatcttggtggtgtctttcaacttgaaaaaggtgaccggctttccgctgaaattaatcggccagattatcttgactttgctgaatccggtcaagtctattttggtattattgctctgggcggtgggcagtacatcaaagctaactccaaattcatcggcatcaccgaactggttcgttcttcttctcgtacgccgtctgataagccggttgcgcacgttgttgcgaacccgcaggcggaggggcaattgcagtggttgaatcgtcgtgcgaacgcgttgttggcgaatggggttgaattgcgtgataaccaattggttgttccgtctgaggggttgtacttgatatattctcaggttttgttcaaagggcaagggtgcccgtctacgcatgttttgttgacgcacacgatatctcgtatagcggtttcttaccagacgaaggttaatttgttgtctgcgataaaatctccgtgtcaacgtgaaacgccggaaggggcggaggcgaagccgtggtatgaaccgatatacttggggggggtttttcagttggaaaaaggggatcgtttgtctgcggagataaaccgtccggactatttggatttcgcggaatctgggcaagtttactttgggataatagcgctgggtggcggattcaacaacttcaccgtttccttctggctgcgcgttccaaaagtttccgcttcccacctggaataa

SEQ ID NO: 53 - protein, 517 aa, Artificial sequence
  3 hTNF monomers joined by tri-glycine linkers and with P2 and P30 epitopes introduced
MVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGGVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGGQYIKANSKFIGITELVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGGFNNFTVSFWLRVPKVSASHLE

SEQ ID NO: 54 - DNA, 1485 bp, Artificial sequence
  3 hTNF joined by tri-glycine linkers and PADRE added C-terminally
atggtgcgcagcagcagccgcacccccagcgacaagcccgtggcccacgtggtggccaacccccaggccgagggccaactgcagtggctgaaccgccgcgccaacgccctgctggcaaacggcgtggagctgcgcgacaaccagctggtggtgcccagcgagggcctgtacctgatctacagccaggtgctgttcaagggccagggctgccccagcacccacgtgctgctgacccacaccatcagccgcatcgccgtgagctaccagaccaaggtgaacctgctgagcgccatcaagagcccctgccagcgcgagacccccgagggcgccgaggccaagccctggtacgagcccatctacctcggcggcgtgttccagctggagaagggcgaccgcctgagcgccgagatcaaccgccccgactacctggacttcgccgagagcggccaggtgtacttcggcatcatcgccctgggtggcggagtccggtcctcctcccggacaccatccgacaaaccagtcgctcatgtcgtcgctaatccacaagctgaaggtcaacttcaatggcttaatcggcgggctaatgctcttcttgctaatggtgtcgaacttcgggacaatcaacttgtcgtcccatccgaaggtctttatcttatttattcccaagtcctttttaaaggtcaaggttgtccatccacacatgtccttcttacacatacaatttcccggattgctgtctcctatcaaacaaaagtcaatcttctttccgctattaaatccccatgtcaacgggaaacaccagaaggtgctgaagctaaaccttggtatgaaccaatttatcttggtggtgtctttcaacttgaaaaaggtgaccggctttccgctgaaattaatcggccagattatcttgactttgctgaatccggtcaagtctattttggtattattgctctgggcggtggggttcgttcttcttctcgtacgccgtctgataagccggttgcgcacgttgttgcgaacccgcaggcggaggggcaattgcagtggttgaatcgtcgtgcgaacgcgttgttggcgaatggggttgaattgcgtgataaccaattggttgttccgtctgaggggttgtacttgatatattctcaggttttgttcaaagggcaagggtgcccgtctacgcatgttttgttgacgcacacgatatctcgtatagcggtttcttaccagacgaaggttaatttgttgtctgcgataaaatctccgtgtcaacgtgaaacgccggaaggggcggaggcgaagccgtggtatgaaccgatatacttggggggggtttttcagttggaaaaaggggatcgtttgtctgcggagataaaccgtccggactatttggatttcgcggaatctgggcaagtttactttgggataatagcgctgggagggggtgccaagttcgtggccgcttggaccctgaaggccgcagcttaa

SEQ ID NO: 55 - protein, 494 aa, Artificial sequence
  3 hTNF joined by tri-glycine linkers and PADRE added C-terminally
MVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGGVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGGVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGGAKFVAAWTLKAAA

SEQ ID NO: 56 - DNA, 1476 bp, Artificial sequence
  3 hTNF joined by tri-glycine linkers and with PADRE in the C-terminus
atggtgcgcagcagcagccgcacccccagcgacaagcccgtggcccacgtggtggccaacccccaggccgagggccaactgcagtggctgaaccgccgcgccaacgccctgctggcaaacggcgtggagctgcgcgacaaccagctggtggtgcccagcgagggcctgtacctgatctacagccaggtgctgttcaagggccagggctgccccagcacccacgtgctgctgacccacaccatcagccgcatcgccgtgagctaccagaccaaggtgaacctgctgagcgccatcaagagcccctgccagcgcgagacccccgagggcgccgaggccaagccctggtacgagcccatctacctcggcggcgtgttccagctggagaagggcgaccgcctgagcgccgagatcaaccgccccgactacctggacttcgccgagagcggccaggtgtacttcggcatcatcgccctgggtggcggagtccggtcctcctcccggacaccatccgacaaaccagtcgctcatgtcgtcgctaatccacaagctgaaggtcaacttcaatggcttaatcggcgggctaatgctcttcttgctaatggtgtcgaacttcgggacaatcaacttgtcgtcccatccgaaggtctttatcttatttattcccaagtcctttttaaaggtcaaggttgtccatccacacatgtccttcttacacatacaatttcccggattgctgtctcctatcaaacaaaagtcaatcttctttccgctattaaatccccatgtcaacgggaaacaccagaaggtgctgaagctaaaccttggtatgaaccaatttatcttggtggtgtctttcaacttgaaaaaggtgaccggctttccgctgaaattaatcggccagattatcttgactttgctgaatccggtcaagtctattttggtattattgctctgggcggtggggttcgttcttcttctcgtacgccgtctgataagccggttgcgcacgttgttgcgaacccgcaggcggaggggcaattgcagtggttgaatcgtcgtgcgaacgcgttgttggcgaatggggttgaattgcgtgataaccaattggttgttccgtctgaggggttgtacttgatatattctcaggttttgttcaaagggcaagggtgcccgtctacgcatgttttgttgacgcacacgatatctcgtatagcggtttcttaccagacgaaggttaatttgttgtctgcgataaaatctccgtgtcaacgtgaaacgccggaaggggcggaggcgaagccgtggtatgaaccgatatacttggggggggtttttcagttggaaaaaggggatcgtttgtctgcggagataaaccgtccggactatttggatttcgcggaatctgggcaagtttactttgggataatagcgctggccaagttcgtggccgcttggaccctgaaggccgcagcttaa

SEQ ID NO: 57 - protein, 491 aa, Artificial sequence
  3 hTNF joined by tri-glycine linkers and with PADRE in the C-terminus
MVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGGVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGGVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALAKFVAAWTLKAAA

SEQ ID NO: 58 - DNA, 1545 bp, Artificial sequence
  3 hTNF joined by glycine linkers and P2 and P30 introduced
atggtgcgcagcagcagccgcacccccagcgacaagcccgtggcccacgtggtggccaacccccaggccgagggccaactgcagtggctgaaccgccgcgccaacgccctgctggcaaacggcgtggagctgcgcgacaaccagctggtggtgcccagcgagggcctgtacctgatctacagccaggtgctgttcaagggccagggctgccccagcacccacgtgctgctgacccacaccatcagccgcatcgccgtgagctaccagaccaaggtgaacctgctgagcgccatcaagagcccctgccagcgcgagacccccgagggcgccgaggccaagccctggtacgagcccatctacctcggcggcgtgttccagctggagaagggcgaccgcctgagcgccgagatcaaccgccccgactacctggacttcgccgagagcggccaggtgtacttcggcatcatcgccctgggtggcggagtccggtcctcctcccggacaccatccgacaaaccagtcgctcatgtcgtcgctaatccacaagctgaaggtcaacttcaatggcttaatcggcgggctaatgctcttcttgctaatggtgtcgaacttcgggacaatcaacttgtcgtcccatccgaaggtctttatcttatttattcccaagtcctttttaaaggtcaaggttgtccatccacacatgtccttcttacacatacaatttcccggattgctgtctcctatcaaacaaaagtcaatcttctttccgctattaaatccccatgtcaacgggaaacaccagaaggtgctgaagctaaaccttggtatgaaccaatttatcttggtggtgtctttcaacttgaaaaaggtgaccggctttccgctgaaattaatcggccagattatcttgactttgctgaatccggtcaagtctattttggtattattgctctgggcggtgggcagtacatcaaagctaactccaaattcatcggcatcaccgaactggttcgttcttcttctcgtacgccgtctgataagccggttgcgcacgttgttgcgaacccgcaggcggaggggcaattgcagtggttgaatcgtcgtgcgaacgcgttgttggcgaatggggttgaattgcgtgataaccaattggttgttccgtctgaggggttgtacttgatatattctcaggttttgttcaaagggcaagggtgcccgtctacgcatgttttgttgacgcacacgatatctcgtatagcggtttcttaccagacgaaggttaatttgttgtctgcgataaaatctccgtgtcaacgtgaaacgccggaaggggcggaggcgaagccgtggtatgaaccgatatacttggggggggtttttcagttggaaaaaggggatcgtttgtctgcggagataaaccgtccggactatttggatttcgcggaatctgggcaagtttactttgggataatagcgctgttcaacaacttcaccgtttccttctggctgcgcgttccaaaagtttccgcttcccacctggaataa

SEQ ID NO: 59 - protein, 514 aa, Artificial sequence
  3 hTNF joined by glycine linkers and P2 and P30 introduced
MVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGGVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALGGGQYIKANSKFIGITELVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALFNNFTVSFWLRVPKVSASHLE

SEQ ID NO: 60 - DNA, 2773 bp, Artificial sequence
  p2ZOp2F expression vector for insect cells
ggatcatgatgataaacaatgtatggtgctaatgttgcttcaacaacaattctgttgaactgtgttttcatgtttgccaacaagcacctttatactcggtggcctccccaccaccaacttttttgcactgcaaaaaaacacgcttttgcacgcgggcccatacatagtacaaactctacgtttcgtagactattttacataaatagtctacaccgttgtatacgctccaaatacactaccacacattgaacctttttgcagtgcaaaaaagtacgtgtcggcagtcacgtaggccggccttatcgggtcgcgtcctgtcacgtacgaatcacattatcggaccggacgagtgttgtcttatcgtgacaggacgccagcttcctgtgttgctaaccgcagccggacgcaactccttatcggaacaggacgcgcctccatatcagccgcgcgttatctcatgcgcgtgaccggacacgaggcgcccgtcccgcttatcgcgcctataaatacagcccgcaacgatctggtaaacacagttgaacagcatctgttcgaatttaaagcttggtaccctcgagctcagctgaattctggatcctctagaccggtcatatgcggccgcggatcgatcgatatctgactaaatcttagtttgtattgtcatgttttaatacaatatgttatgtttaaatatgtttttaataaattttataaaataatttcaacttttattgtaacaacattgtccatttacacactcctttcaagcgcgtgggactcgatgctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgatgataaacaatgtatggtgctaatgttgcttcaacaacaattctgttgaactgtgttttcatgtttgccaacaagcacctttatactcggtggcctccccaccaccaacttttttgcactgcaaaaaaacacgcttttgcacgcgggcccatacatagtacaaactctacgtttcgtagactattttacataaatagtctacaccgttgtatacgctccaaatacactaccacacattgaacctttttgcagtgcaaaaaagtacgtgtcggcagtcacgtaggccggccttatcgggtcgcgtcctgtcacgtacgaatcacattatcggaccggacgagtgttgtcttatcgtgacaggacgccagcttcctgtgttgctaaccgcagccggacgcaactccttatcggaacaggacgcgcctccatatcagccgcgcgttatctcatgcgcgtgaccggacacgaggcgcccgtcccgcttatcgcgcctataaatacagcccgcaacgatctggtaaacacagttgaacagcatctgttcgaattaattcggatctctgcagcacgtgttgacaattaatcatcggcatagtatatcggcatagtataatacgactcactataggagggccaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgaccgacgccgaccaacaccgccggtccgacggcggcccacgggtcccaggggggtcgacctcgaaacttgtttattgcagcttataatggttacaaataaagcaatagcatcacaaatttcacaaataaagcatttttttcactgcattctagttgtggtttgtccaaactcatcaatgtatcttatcatgtct

(19) United States
(12) Patent Application Publication
(10) Pub. No.: US 2005/0180947 A1
(43) Pub. Date: Aug. 18, 2005

(54) Novel application of vaccination against TNF-alpha

(76) Inventors: Hans Rudolf Pedersen, Valby (DK); Bjarke Ebert, Valby (DK); Louise Henriette Pedersen, Valby (DK); Peter Birk Rasmussen, Horsholm (DK)

Correspondence Address: FROMMER LAWRENCE & HAUG, 745 FIFTH AVENUE- 10TH FL., NEW YORK, NY, 10151, US

(21) Appl. No.: 10/939,107

(22) Filed: Sep. 10, 2004

Related U.S. Application Data
(63) Continuation-in-part of application No. PCT/DK03/00147, filed on Mar. 11, 2003.
(60) Provisional application No. 60/363,128, filed on Mar. 11, 2002.

(30) Foreign Application Priority Data
Mar. 11, 2002 (DK) PA 2002 00368

(51) Int. Cl. A61K 38/19; A61K 48/00

(52) U.S. Cl. 424/85.1; 514/44

(57) ABSTRACT

The present invention relates to novel medical applications of down-regulation of tumour necrosis factor α (TNF-α) activity, especially novel applications of active immunization against TNF-a in order to reduce or alleviate pain. In particular, the present invention discloses novel methods for treating or ameliorating neuropathic pain.

### REFERENCE TO RELATED APPLICATIONS

This application is a continuation-in-part of international patent application PCT/DK03/00147 filed Mar. 11, 2003 and published as WO 03/07591 on Sep. 18, 2003, which claims priority from Danish Patent Application PA 2002 00368 filed Mar. 11, 2002, and U.S. Provisional Application 50/363,128 filed Mar. 11, 2002. Reference is also made to jointly-owned international application number PCT/DK02/00764 which was published as WO 03/042244, the contents of which, including the sequence listing, are incorporated herein by reference.

Indeed, each of these applications, and each application and patent mentioned in this document, and each document cited or referenced in each of the above applications and patents, including during the prosecution of each of the applications and patents (“application cited documents”) and any manufacturer's instructions or catalogues for any products cited or mentioned in each of the applications and patents and in any of the application cited documents, are hereby incorporated herein by reference. Furthermore, all documents cited in this text, and all documents cited or referenced in documents cited in this text, and any manufacturer's instructions or catalogues for any products cited or mentioned in this text, are hereby incorporated herein by reference.

It is noted that in this disclosure, terms such as “comprises”, “comprised”, “comprising”, “contains”, “containing” and the like can have the meaning attributed to them in U.S. Patent law; e.g., they can mean “includes”, “included”, “including” and the like. Terms such as “consisting essentially of” and “consists essentially of” have the meaning attributed to them in U.S. Patent law, e.g., they allow for the inclusion of additional ingredients or steps that do not detract from the novel or basic characteristics of the invention, i.e., they exclude additional unrecited ingredients or steps that detract from novel or basic characteristics of the invention, and they exclude ingredients or steps of the prior art, such as documents in the art that are cited herein or are incorporated by reference herein, especially as it is a goal of this document to define embodiments that are patentable, e.g., novel, nonobvious, inventive, over the prior art, e.g., over documents cited herein or incorporated by reference herein. And, the terms “consists of” and “consisting of” have the meaning ascribed to them in U.S. Patent law; namely, that these terms are closed ended.

### FIELD OF THE INVENTION

The present invention relates to novel medical applications of down-regulation of tumour necrosis factor α (TNF-α) activity, especially novel applications of active immunization against TNF-α in order to reduce or alleviate pain.

### BACKGROUND OF THE INVENTION

Vaccines against autologous antigens have traditionally been prepared by “immunogenizing” the relevant self-protein, e.g. by chemical coupling (“conjugation”) to a large foreign and immunogenic carrier protein (cf. U.S. Pat. No. 4,161,519) or by preparation of fusion constructs between the autologous protein and the foreign carrier protein (cf. WO 86/07383). In such constructs, the carrier part of the immunogenic molecule is responsible for the provision of epitopes for T-helper lymphocytes (“T_{H} epitopes”) that render possible the breaking of autotolerance.

Later research has proven that although such strategies may indeed provide for the breaking of tolerance against autologous proteins, a number of problems are encountered. Most important is the fact that the immune response that is induced over time will be dominated by the antibodies directed against the carrier portion of the immunogen whereas the reactivity against the autologous protein often declines, an effect that is particularly pronounced when the carrier has previously served as an immunogen—this phenomenon is known as carrier suppression (cf. e.g. Kaliyaperumal et al. 1995., Eur. J. Immunol 25, 3375-3380). However, when using therapeutic vaccination it is usually necessary to re-immunize several times per year and to maintain this treatment for a number of years and this also results in a situation where the immune response against the carrier portion will be increasingly dominant on the expense of the immune response against the autologous molecule.

Further problems involved when using hapten-carrier technology for breaking autotolerance is the negative steric effects exerted by carrier on the autologous protein part in such constructs: The number of accessible B-cell epitopes that resemble the conformational patterns seen in the native autologous protein is often reduced due to simple shielding or masking of epitopes or due to conformational changes induced in the self-part of the immunogen. Finally, it is very often difficult to characterize a hapten-carrier molecule in sufficient detail.

WO 95/05849 provided for a refinement of the above-mentioned hapten-carrier strategies. It was demonstrated that self-proteins wherein is in-substituted as little as one single foreign T_{H} epitope are capable of breaking tolerance towards the autologous protein. Focus was put on the preservation of tertiary structure of the autologous protein in order to ensure that a maximum number of autologous B-cell epitopes would be preserved in the immunogen in spite of the introduction of the foreign T_{H} element. This strategy has generally proven extremely successful inasmuch as the antibodies induced are broad-spectred as well as of high affinity and that the immune response has an earlier onset and a higher titer than that seen when immunizing with a traditional carrier construct.

WO 00/20027 provided for an expansion of the above principle. It was found that introduction of single T_{H} epitopes in the coding sequence for self-proteins could induce cytotoxic T-lymphocytes (CTLs) that reacts specifically with cells expressing the self-protein. The technology of WO 00/20027 also provided for combined therapy, where both antibodies and CTLs are induced—in these embodiments, the immunogens would still be required to preserved a substantial fraction of B-cell epitopes.

WO 95/05849 (relating to a generally applicable technology) and WO 98/46642 (relating to vaccination of humans) disclose vaccine technology that is suitable for down-regulating the activity of TNF-α (tumour necrosis factor α), a cytokine involved in the pathology of several diseases such as type I diabetes, rheumatoid arthritis, and inflammatory bowel disease. Both disclosures teach preservation of the tertiary structure of monomer TNF-α when this molecule confronts the immune system.

Neuropathic Pain

Normally the capacity to experience pain has a protective role. It helps one to realize imminent danger of tissue damage, or in the case of actual damage, establishes a profound but reversible pain hypersensitivity in the inflamed and surrounding tissue. The pain has a physiological purpose because it supports wound repair, in terms of preventing any contact with the damaged part until healing has occurred. Prolonged chronic pain on the other hand, is a pain without any physiological purpose and must therefore be regarded as pathological (Millan, 1999).

Aetiology and Symptoms of Neuropathic Pain

Chronic pain typically results from damage to the nervous system—the peripheral nerve, the dorsal root ganglion, dorsal root, or the central nervous system. The pathological response to such damage is termed neuropathic pain (Woolf et al.; 1999). The aetiology of neuropathic pain is very wide, ranging from peripheral or cranial nerve trauma, postherpetic neuralgia, HIV-associated neuralgia, neoplasia, diabetic neuropathy to stroke (Nicholson, 2000). Despite this variety in aetiology, neuropathic pain conditions share certain clinical characteristics, such as spontaneous, continous pain, burning sensations, paroxysmal pain (shooting, lancinating pain), evoked pain to various mechanical or thermal stimuli, such as allodynia and hyperalgesia, and sensory loss in the painful area (Attal et al., 1999).

Mechanisms of Peripheral Neuropathic Pain—Peripheral

Primary afferent input to the dorsal horn is, under normal physiologic conditions, mediated by three main classes of cutaneous primary afferent neurons: C, Aδ and Aβ fibres. C fibres are thin, unmyelinated and slowly conducting. Aδ fibres are medium sized, myelinated and of intermediate velocity. Aβ fibres are large, myelinated and fast conducting. Upon exposure to the skin of a noxious stimuli, Aδ fibres elicit a sharp and rapid first phase of pain, followed by a more “dull” pain evoked by the C fibres. These two classes of neurons are referred to as nociceptors. Aβ fibres (mechanoreceptors) only respond to innocuous mechanical stimuli such as touch, vibration or pressure. All three classes of fibres can transmit non-nociceptive information (Millan, 1999).

Several clinical observations suggest that the initial mechanism of pain after a peripheral nerve injury may be initiated by functional alterations of primary afferent nociceptors. The major mechanisms identified, for the generation of abnormal activity in primary afferents, are ectopic activity, fibre interactions, sensitization of nociceptors and catecholamine hypersensitivity (Attal et al., 1999).

Ectopic Discharges

When a peripheral nerve is lesioned, the damaged axons of both myelinated and unmyelinated fibres start to sprout. These sprouts develop an abnormal spontaneous electrical activity and also enhance the sensitivity of various mechanical thermal and chemical stimuli (Scadding, 1981). Since the abnormal activity does not originate from the nociceptors, these discharges have been termed ectopic. Ectopic discharges have been demonstrated in models of partial nerve injury (Kajander et al., 1992) and in the experimental neuroma (Wall, 1974). Ectopic discharges may also arise from local patches of demyelination and somatas in the dorsal root ganglion (Attal et al., 1999).

This type of abnormal activity is basically believed to arise through an accumulation of sodium channels, in the axon at the neuroma site and along the axon. There are two types of sodium channels on the sensory neurons. A type that is sensitive to tetrodotoxin, found on all sensory neurons and a type that is insensitive to tetrodotoxin, which is only found on the nociceptor sensory neurons. Generation of action potentials and thereby neuronal firing is dependent on these voltage gated sodium channels. After peripheral nerve injury, expression of both types of sodium channels is highly upregulated. This is believed to result in lowered action potential thresholds leading to hyperexcitability and ectopic discharge, all contributing to ongoing pain (Waxman et al., 2000).

Fiber Interactions—Ephapses and Cross-Exitations

Normally individual afferents conduct impulses essentially independent from one another. But under pathological conditions, such as nerve injury, abnormal electrical connections can occur between adjacent demyelinated axons. These are referred to as ephapses. “Ephaptic cross talk” may result in the transfer of nerve impulses from one axon to another. Cross talk between low-threshold A fibres and high-threshold C fibres develops in the dorsal root ganglion and may represent a way for the non-nociceptive afferents to activate the nociceptors (Amir et al., 2000). A similar event referred to as “crossed afterdischarge” has also been demonstrated in animals. Here repetitive synchronous activity in primary afferents induces autonomous firing in neighbours. This is a very early event after nerve injury, it involves a large portion of afferents and it does not depend on structural alterations (Lisney et al., 1987).

Nociceptor Sensitization

In the presence of inflammation, nociceptors acquire new characteristics and are said to be sensitized. This sensitization in nociceptors is characterized by a spontaneous discharge, a lowered threshold to activation stimuli, and an abnormal discharge to suprathreshold stimulation (noxious stimulus causes more pain than normal, a condition called hyperalgesia) (Besson et al., 1987). One of the primary causes for this sensitization is believed to be inflammation. Following peripheral nerve injury, the inflammatory cells are activated leading to vasodilation and extravasation of plasma proteins, accompanied by release of chemical mediators. This long line of chemical mediators include serotonin, bradykinin, substance P, histamine, prostaglandins, purines, cytokines, leucotrienes, nerve growth factor and neuropeptides. The end result is a chemical sensitization of high-threshold nociceptors to transmit low-intensity painful stimuli (Nicholson, 2000).

Involvement of the Sympathetic Nervous System

Neurophatic pain may also be sympathetically maintained. After nerve injury, injured and nearby uninjured axons begin to express α-adrenoreceptors. The α-adrenoreceptors leave the axons sensitive to catecholamine stimulation, both from the circulating catecholamines and from norepineprine released from postganglionic sympathetic terminals (Woolf et al., 1999). In addition sympathetic neurons sprout around dorsal root ganglion cells, forming “basket-like” structures. This may constitute a mechanism in which sympathetic activity initiates activity in sensory fibres (McLachlan et al., 1993). These abnormal interactions of sympathetic fibres have also been proposed as a mechanism for the generation of ectopic impulses (Kim et al., 1997).

Mechanisms of Peripheral Neuropathic Pain—Central

A range of changes is also seen in the central nervous system. These changes can be narrowed down to three major types of modifications, alteration of pain modulatory control systems, structural modifications and central sensitization (Attal et al., 1999).

Alteration of Pain Modulatory Control Systems

Neurons in the dorsal horn of the spinal cord receive input from primary afferents. Dorsal horn neurons then proces and transfer information about peripheral stimuli to the brain. Their firing is not only determined by excitatory input, but also by the inhibitory inputs they receive locally from the spinal cord and descending from the brain. An increased inhibition of dorsal root neurons results in reduced activity and acts as a spinal “gate” (Woolf et al., 1999).

There are currently no data on modifications of inhibitory descending systems, but several studies have suggested local reduction in inhibition at the spinal cord level. A decrease in the dorsal root potential has been observed after nerve section or constriction, indicating a reduction of presynaptic inhibitions (Laird et al., 1993; Wall et al., 1981). A decrease in postsynaptic inhibition of wide dynamic range dorsal horn neurons (WDR) by primary afferent Aβ fibres after peripheral nerve section has also been observed (Woolf et al., 1982). This decrease in postsynaptic inhibition of WDR neurons is thought to arise through a loss of Aβ fibre stimulation of inhibitory interneurons and a reduced functionality of the inhibitory interneurons on the WDR neurons (Millan, 1999).

GABA and glycine act as inhibitory neurotransmitters in the dorsal horn, and a decrease of GABA and glycine levels have been observed in the spinal dorsal horn after peripheral nerve injury (Castro-Lopez et al., 1993). Also GABA receptors and opioid receptors, which exist both presynaptically on primary sensory neurons and postsynaptically on dorsal horn neurons, are downregulated. All together these changes in inhibitory control systems lead to increased excitability of dorsal horn neurons, in manner of increased response to both excitatory and spontaneously firing of dorsal horn neurons (Woolf et al., 1999).

Structural Modifications

A phenomena of Aβ fibre sprouting into the superficial layers of the doral horn, after a peripheral nerve injury, represents a mechanism for non-noxious stimuli to generate a noxious response. Normally the Aβ fibres terminate in all of the laminas of the dorsal horn except for lamina II₀, which are only innervated by C and Aδ fibres. After a peripheral nerve injury, atrophy of C fibre termination in lamina II₀ occurs and Aβ fibre terminals start to sprout into this lamina. Receiving information of non-noxious stimuli in a lamina that normally only processes painful stimuli may lead to misinterpretation of stimuli by the nervous system (Millan, 1999). It has been suggested that growth factors, neurotrophins or possibly neuropeptides released by injured C fibres may play a role in this central reorganization (Attal et al., 1999).

Central Sensitization

C fibres release glutamate as their neurotransmitter. The spinal cord neurons that receive input from C fibres express three subtypes of glutaminergic receptors. Namely the NMDA and the AMPA/kainate receptors (ionotropic) and the metabotropic receptors (G-protein coupled). Normally glutamate released from C fibres induces depolarization of dorsal horn neurons after binding to non-NMDA receptors. Under pathological conditions the prolonged activation of C fibres evokes excessive release of glutamate that activates the NMDA receptors (Bennett, 2000).

Under physiological conditions, the NMDA receptor channel is blocked at resting potential in a voltage-dependent manner by binding of Mg++ inside the ion channel. Activation of the NMDA receptors is induced by removal of this block. This subsequently induces a massive influx of calcium within the cell, which provokes a cascade of intracellular events leading to long-lasting modification of the properties of the dorsal horn neurons (Attal et al., 1999).

Following activation of the NMDA receptor, these modifications in the dorsal horn neuron induce a change in the sensitivity of the postsynaptic cell causing it to respond more strongly to all of its inputs (including inputs from Aβ fibres). This effect is called central sensitization. It is believed that C-nociceptor sensitization caused by tissue inflammation or by spontaneously discharge, due to nerve injury, can produce prominent and long-lasting central sensitization (Bennett, 2000).

One of the intracellular events is an upregulation of phospholipase C levels that leads to production of prostaglandins. A proposed mechanism for the spread of central sensitization is that diffusable prostaglandins spread and thereby increase the excitability of adjacent neurons and expand the receptive field size (MacFarlane et al., 1997). The protein products of proto-oncogenes c-fos and c-jun may also be important in the prolonged sensitization process. A persistent increase in fos expression has been shown following sciatic nerve transsection (Chi et al., 1993).

Neuropeptides such as substance P and calcitonin-gene-related peptide (CGRP) also seem to be involved in the central sensitization. They are normally expressed by Aδ and C fibres and are strongly implicated in the sensory transmission between nociceptors and the central nervous system. After peripheral nerve injury, nociceptor expression of these neuropeptides is downregulated. Instead Aβ fibres begins to express these neuropeptides (a phenotypic switch) and low threshold stimuli may cause release of substance P in the dorsal horn neurons, generating a state of central hyperexcitability (Miki et al., 1998).

Pharmacologic and Clinical Approaches to Neuropathic Pain

Currently, there are no truly effective treatments against development of neuropathic pain or against neuropathic pain. Taking just a glance at the complex mechanisms, peripheral or central, outlined in the sections above, this may not seem so strange. These mechanisms do not occur in a domino-effect order, and a patient may exhibit all from a wide range of symptoms down to just a single one. Suitable animal models for chronic and inflammatory pain have only been known in little over a decade. And even though there has been a substantial amount of research in the field and many of the possible mechanisms have been elucidated, there is still very little knowledge about the effect of neuropathic pain at the supraspinal level.

Hyperalgesia and Allodynia

In neuropathic pain states, the two key features are hyperalgesia and allodynia. The term hyperalgesia refers to an exaggerated response to painful stimuli and allodynia refers to pain evoked by normally non-painful stimuli. Hyperalgesia and allodynia are both descriptions of clinical symptoms, and do not imply a mechanism. Several distinct mechanisms account for the manifestation of the symptoms. Interpretation of research results, regarding these symptoms, should therefore rely on considerations of the different mechanistical pathways and not just on the presence/absence of these symptoms.

Cutaneous tissue damage is associated with two principal zones of pain, primary hyperalgesia and secondary hyperalgesia. The zone of primary hyperalgesia comprises the region of tissue damage itself, whereas the zone of secondary hyperalgesia refers to the surrounding undamaged zone (Millan, 1999).

Primary Hyperalgesia

Primary hyperalgesia is characterized by spontaneous pain and increased sensitivity to heat (thermal hyperalgesia), mechanical and chemical stimuli. It may involve a contribution of processes integrated in the central nervous system, but can predominantly be explained by changes at the peripheral nociceptor level (Millan, 1999). The increased responsiveness to heat stimuli appears to involve an enhanced sensitivity of individual peripheral nociceptors. The class of nociceptors involved depends on the skin type, whether it is hairy or non-hairy (glabrous). In hairy skin, C-fibres are mainly involved whereas in non-hairy skin, Aδ fibres are predominantly involved (Treede et al., 1992).

The processes underlying the activation and sensitization of primary afferent nociceptor terminals are highly complex and involve substances derived from damaged tissue, immune competent cells, the vasculature, sympathetic terminals and from the nociceptors themselves. These substances include bradykinin, serotonine, prostaglandins, protons, cytokines and Nerve Growth Factor. The alteration of primary afferent nociceptor function may ultimatively be mediated by alterations of the ion channels controlling their activity (Millan, 1999).

Secondary Hyperalgesia and Allodynia

Secondary hyperalgesia displays an increase in sensitivity to mechanical stimuli, but not to heat stimuli. The key feature of secondary hyperalgesia is mechanical allodynia and the mechanisms predominantly involved are central. There are two forms of mechanical allodynia, namely dynamic hyperalgesia (also named dynamic allodynia) and punctate (static) allodynia. The first form is evoked by normally innocuous, mechanical stimulation, such as light touch or brushing of the skin. This type of allodynia is primarily mediated by Aβ fibres. Punctate allodynia is elicited by non-noxious, localized, mechanical stimuli, such as application of von Frey hairs. Both forms require the induction of central sensitization for their manifestation, but punctate allodynia develops more rapidly, is more persistent and occupies a larger tissue region than its dynamic counterpart (Millan, 1999). The precise mechanisms underlying punctate allodynia are yet not known. Proposed theories involve both C fibres (Cervero et al., 1994) and Aδ fibres (Ali et al., 1996). One of the latest studies (Ziegler et al., 1999) conclude secondary hyperalgesia, due to punctate stimuli, to be induced by nociceptive C fibre discharge, but to be mediated through nociceptive Aδ fibres.

Cold Allodynia

Cold allodynia refers to the induction of pain by normally non-noxious cold stimuli. This abnormal responsiveness to cold is seen in regions of secondary hyperalgesia, especially in areas innervated by damaged nerves. Cold allodynia can be distinguished from the reduction in pain that can be provided by cooling the area of primary hyperalgesia (Millan, 1999). Cold allodynia appears to be mediated by C fibres interacting with sensitized wide dynamic range neurons in the dorsal horn (Cervero et al., 1994).

Animal Models of Neuropathic Pain

During the last few years, pain due to peripheral nerve injury has been the subject of fundamental basic and clinical research. This has lead to extensive progress in the understanding of the pathophysiological processes underlying neuropathic pain. The improved knowledge of neuropathic pain is largely due to the development of animal models of peripheral, traumatic, metabolic and toxic nerve injuries.

One of the earliest attempts to produce an animal model of chronic pain was the model of experimental neuroma (Wall et al., 1974). This model was based on a complete nerve transsection, and even though it produced spontaneous pain, it could not mimic the clinical observations of hyperalgesia and allodynia seen in man. In the search for models able to produce the same disorders as those seen in man, Bennett and Xie developed the Chronic Constriction Injury (CCI) model (Bennett and Xie, 1988). More soon followed this model and today a wide variety of models exist.

The focus in the following sections will be on models addressing peripheral nerve injury. However, there are other models, including Streptozotocin-induced diabetic neuropathy (Wuarin-Bierman et al., 1987), Vincristine-induced neuropathy (Aley et al., 1996) used for the study of chemotherapy induced neuropathy, experimental neuritis of the rat sciatic nerve (Eliav et al., 1999) used for study of the inflammatory role of neuropathic pain, and Spinal nerve ligation (SNL) used for the study of neuropathic pain disorders (Kim et al., 1992).

Chronic Constriction Injury

This model (CCI), presented by Bennett and Xie in 1988, is still one of the most used models for studies of neuropathic pain. Mainly because it is reliable and easy to reproduce. The model builds on a constriction of the rat sciatic nerve, just proximal to the trifurcation of the nerve. Four loosely tied ligatures of chromic gut are placed with 1 mm spacing, around the nerve, comprising a total of 7 mm of the nerve. The compression of the nerve is thought to be a consequence of endoneurial edema. The endoneurial edema might result from decreased epineural blood flow and venous stasis (Sommer et al., 1993). Edema causes an elevation of endoneurial fluid pressure, which in combination with the decreased blood flow furthers degeneration of the nerve (Myers et al., 1991).

Post mortem procedures reveal severe demyelination of the injured region. Sham operations can be conducted contralaterally, and because pain symptoms develop unilaterally, the animal can serve as its own control. Hyperalgesia measured as noxious heat is seen from the second day post surgery, and reaches a maximum 8-10 days post surgery (Bennett and Xie, 1988).

The initial studies failed to find evidence of mechanical allodynia, but later studies with von Frey hairs showed substantial punctate allodynia from the second day post surgery, increasing to a maximum within few days (Bennett, 1998). Other observed symptoms include cold allodynia, hind paw guarding, abnormalities of skin temperature and advanced abnormal claw growth on the side of the nerve damage (Bennett and Xie, 1988). Interestingly, the feature of abnormal nail growth is also seen in human causalgia, and patients often let the nails grow overlong, explaining that trimming them is painful (Bennett, 1998).

Thermal hyperalgesia and mechanical allodynia can be observed for more than two months after surgery (Bennett and Xie, 1988), making the CCI model very useful for long term studies, or even for multiple small studies.

There are however some drawbacks to this model. The original study reported autotomy in 70% of the animals, seen as gnawing of claw tips, down to the root of the claw, causing the claw to bleed. The autotomy was most commonly seen within the first ten days post surgery (Bennett and Xie, 1988). This seems however not to be a widely reported problem today, a guess, from this author would be that refinement of ligature placement has developed over the years.

A more recently addressed drawback, is that the CCI model introduces foreign material into the wound. The release of chemicals from the chromic gut ligatures causes a local inflammatory reaction. This makes distinction between the neuropathic and the inflammatory component difficult (Lindenlaub and Sommer, 2000). Some authors have even suggested that the chemicals released from ligatures is the main factor causing hyperalgesia in CCI (Maves et al., 1993; Clatworthy et al., 1995). However, seen in the light of a new model, circumstantial evidence rejects this theory (Lindenlaub and Sommer, 2000).

Partial Sciatic Nerve Ligation

The model of partial sciatic nerve ligation (PSL) was developed by Seltzer, Dubner and Shir (Seltzer et al., 1990). This model has also been widely used for studies of neuropathic pain. The model builds on a tight partial constriction of the rat sciatic nerve, just distal to the branch of the posterior biceps semitendinosus (PBST) nerve. An 8-0 silicon-treated silk suture is inserted into the nerve and tightly ligated, so that the dorsal ⅓ to ½ of the nerve is trapped in the ligature.

The site for the ligation, just distal to the PBST nerve is very important for the obtaining of reliable results, which makes the model a bit more difficult to perform than the CCI model. The initial study (Seltzer et al., 1990) showed that ligature insertion several mm down (from the PBST nerve), to a point proximal of the trifurcation, produced unpredictable and highly variable data. Schmalbruch has shown that the sciatic becomes fasciculated into its main branches just distal to a small fat pad a few mm from the PBST branch point (Schmalbruch, 1986). Therefore, the location of the sciatic injury has to be between the PBST descendence from the sciatic and this small fat pad (Seltzer et al., 1990).

Hyperalgesia measured as noxious heat and mechanical allodynia measured with von Frey hairs can be seen as early as 1 day post surgery. Other observed symptoms include signs of spontaneous pain, an exaggerated response to suprathreshold heat stimuli, hind paw guarding, and exaggerated response to noxious mechanical stimuli (pin prick test). Cold allodynia and autotomy were not observed (Seltzer et al., 1990).

One interesting feature of the PSL is that some of the pain symptoms develop bilaterally. A marked contra lateral reduction in threshold to both heat stimuli, pin prick test, and mechanical allodynia has been observed (Seltzer et al., 1990). This bilateral tendency of course excludes the use of animals as their own controls. It also implies that the PSL model depends critically on sympathetic outflow. This has been supported by further studies, where sympathectomy to some extend alleviates pain behaviour (Kim et al., 1997; Shir and Seltzer, 1991).

The duration of hyperalgesia and allodynia was reported to be at least 54 days in the original study (Seltzer et al., 1990). However, in the studies reported herein, both hyperalgesia and allodynia responses were less pronounced two weeks post surgery. This may though be due to imperfect surgical skills of the experimenters. The model is in general thought to be less severe than the CCI model, and the rapid onset of both hyperalgesia and allodynia makes it very useful for short-term studies.

Some of the drawbacks to the PSL model are the same as those seen in the CCI model. Even though the inflammatory component of the PSL model is less pronounced than in the CCI model, the introduction of foreign material into the wound still causes a local inflammatory reaction. This makes it difficult to distinct between the neuropathic and inflammatory component (Lindenlaub and Sommer, 2000). Another drawback to the PSL model is that damaged and undamaged primary afferents are mixed in the nerve. This makes studies of the changes in dorsal root ganglion difficult (Walker et al., 1999).

Partial Sciatic Nerve Transsection

A fairly new model of peripheral nerve injury has been presented by Lindenlaub and Sommer (Lindenlaub and Sommer, 2000). The model of partial sciatic nerve transsection (PST) was first described by Ma and Bisby (Ma and Bisby, 1998). They did, however, not investigate the model for pain related behaviour, but used the model for a study of peptide expression in the dorsal root ganglion neurons. The standardization of the model, and the characterization of pain related behaviour were conducted by Lindenlaub and Sommer.

The model builds, as the name implies, on a partial transsection of the rat sciatic nerve. The nerve is exposed at a mid-thigh level, and a 7-0 prolene ligature is inserted through the midpoint of the nerve, just cranially to the branch running to musculus biceps femoris. Half of the nerves diameter is transected in a ventrocranial direction up to the ligature, the ligature is thereafter removed. Post mortem procedures showed endoneurial edema, massive degeneration of myelinated fibre and an increase of the endoneurial cells in the sciatic nerve. Epineurial inflammation is not observed (Lindenlaub and Sommer, 2000).

The PST model shows thermal hyperalgesia and mechanical allodynia, due to punctate stimuli, from the second day post surgery. These symptoms can be observed until post surgery 41 and 48 days, respectively, and the magnitude in threshold responses is comparable to those seen in the CCI. The interindividual time course of animals is however very variating. Other observed symptoms include hind paw guarding. Autotomy is not observed (Lindenlaub and Sommer, 2000).

Apart from the interindividual variation, which may cause comparative problems in analysis of data, this model seems very promising, mainly because it represents a pure nerve injury, without the introduction of foreign material into the wound. It comprises a way to study the neuropathic pain component without additional epineural inflammation.

Lindenlaub and Sommer suggest that the model could be used for investigation of the pathogenic role of cytokines and other inflammatory molecules in the induction of neuropathic pain (Lindenlaub and Sommer, 2000).

Cytokines in Neuropathic Pain

Cytokines are a heterogeneous group of polypetides that were originally described to mediate and regulate activation of the immune system and inflammatory responses. They are expressed in numerous tissues, including the peripheral and central nervous systems. Cytokines have an extremely wide range of actions and have therefore been termed pleiotropic. When subdividing cytokines into groups, one differentiates between pro-inflammatory cytokines and anti-inflammatory cytokines, according to their action on immune cells.

Pro-Inflammatory Cytokines and Their Effects on the Immune System

The immune system's defence against foreign pathogens and foreign self can be divided into following responses. The first response is called the innate (non-specific) immune response, and is mainly mediated through phagocytosis/endo-cytosis and inflammation. The second response is called the adaptive (specific) immune response, and this response can be further subdivided into the cell-mediated and the humoral responses. These responses are mainly mediated by T-cells and B-cells respectively, the later expressing and producing antibodies against the foreign pathogen/self (Janeway et al., 1999).

Local Effects of Pro-Inflammatory Cytokines

Following an infection, surface receptors on the phagocytes (neutrophils and macrophages) bind the bacterial molecules. This triggers the phagocytes to engulf the bacteria and induces the secretion of biologically active molecules by these phagocytes. Among these molecules are the cytokines, and their effects, in response to bacterial components, are commonly known as inflammation (Janeway et al., 1999).

The term inflammation is defined as heat, pain, redness and swelling, and reflects the effects of cytokines on the local blood vessels. The cytokines induce vasodilation, which leads to increased local blood flow, and they also increase the permeability of the blood vessels, allowing fluid and proteins to pass into the infected tissue. Circulating neutrophils and macrophages also migrate from the blood vessels to the site of infection, furthering the release of inflammatory mediators. The accumulation of the fluid and cells at the site of inflammation causes the redness, heat, swelling and pain (Janeway et al., 1999).

Systemic Effects of Pro-Inflammatory Cytokines

The above-described effects of pro-inflammatory cytokines are local, but the cytokines released by phagocytes also have many systemic effects, that contribute to the host defence. Some of the cytokines act as endogenous pyrogens, causing fever. Fever is an important part of the host defence, since most pathogens grow better at lower temperatures and adaptive immune responses are more intense at raised temperatures (Janeway et al., 1999).

Another systemic effect of the cytokines is the induction of the acute-phase response. This response involves the production and secretion of acute-phase proteins from the liver into the blood stream. These proteins are generated within a day or two following infection, and have functional properties as antibodies, binding to a broad range of bacteria. They do however lack the specificity of antibodies, since they have no structural diversity (Janeway et al., 1999).

A final important effect of the cytokines is to induce leukocytosis, an increase in the circulating neutrophils, and to promote the maturation of dendritic cells into antigen-presenting cells. These cells are crucial for the initiation of the adaptive immune response. All together the pro-inflammatory cytokines contribute to the control of infection while the adaptive immune response develops (Janeway et al., 1999).

Tumour Necrosis Factor α—General Aspects

TNF-α is a 17-26 kDa protein consisting of 185 amino acids, and it is synthesized as a precursor protein of 212 amino acids. It is found as both soluble and membrane-bound forms, the active form usually being a homotrimer (Janeway et al., 1999). TNF-α exerts its action mainly through the two TNF-receptors, TNF receptor I (renamed CD120a) and TNF receptor II (renamed CD120b). The majority of the TNF-α effects are transmitted through CD120a, whereas the CD120b receptor is inducible and preferentially reacts with membrane bound TNF-α. (Tartaglia et al., 1992, Vandenabeele et al., 1995, Grell et al., 1995).

Besides the actions of TNF-α mentioned in the sections above, TNF-α also plays an important role in the containment of a local infection. TNF-α induces blood clotting in the local small vessels, occluding them, thereby cutting off the blood flow. This prevents pathogens from spreading into the bloodstream, infecting other parts of the body. If however infection escapes into the bloodstream, a phenomena known as sepsis, TNF-α is released systemically causing vasodilation and loss of plasma volume, leading to shock. Septic shock is also triggered by TNF-α, leading to generation of clots in the small vessels and the massive consumption of clotting proteins. The lack of normal perfusion of the liver, heart, lungs and kidneys quickly leads to failure of these vital organs, and the consequences are often fatal (Janeway et al., 1999).

Brain Derived TNF-α Mediates Neuropathic Pain

It is known that TNF-α acts in the development of persistent pain, but TNF-α also appears to be a crucial factor in the conscious perception of pain. It has been shown that following surgery of the Chronic Constriction Injury (CCI) model on male Spraque-Dawley rats, bioactive levels of TNF-α in the locus coeruleus (LC) and in the hippocampus were assayed. This revealed a significant increase of TNF-α levels in the LC 4 days post surgery. Furthermore, at 8 days post surgery, a significant increase was seen in both LC and hippocampus. The increase in TNF-α levels in the hippocampus occurs concomitant with the time course of symptom development in this model. Constitutive expression of TNF-α in the LC was also shown in control groups, and by day 14 after surgery, the TNF-α levels in the LC and hippocampus of the CCI group had returned to this baseline constitutive expression. The return to baseline levels interestingly occurred at the same time as symptoms of neuropathic pain resolved (Covey et al., 2000).

Another study using continuous intracerebroventricular 15 (i.c.v.) administration of recombinant racine α (rr-TNF-α) showed a similar involvement of TNF-α in the mediation of pain (Ignatowski et al., 1999). I.c.v. microinfusion of rr-TNF-α on CCI rats displayed an enhancement of hyperalgesia, and in unoperated rats i.c.v. microinfusion of rr-TNF-α even induced hyperalgesia. Bioassays were used to determine TNF-α levels in the lumbar spinal cord, in the systemic circulation and in the hippocampus. These showed no changes in TNF-α levels in the lumbar spinal cord or the systemic circulation following i.c.v. infusion, but increased TNF-α levels in the hippocampus were observed. This strongly indicates that the action of TNF-α occurs at brain centres that modulate pain perception.

To further test this effect of TNF-α on pain related behaviour, mono- and polyclonal antibodies against TNF-α have been i.c.v. infused following CCI. The antibodies were infused from 4 and 6 days post-CCI, and hyperalgesia was measured as reduction in withdrawal responses to thermal stimuli. Polyclonal antibody infusion starting at day 4 completely abolished the hyperalgesic response by day 6, with hypoalgesic responses days 8 to 12, thereafter showing significant differences in thresholds from control CCI groups until day 16. Infusion of polyclonal antibody starting day 6 showed no effect, indicating a strong time-dependency for the TFN-α effect in the brain. This finding could be due to an induction of the cytokine cascade e.g. TFN-α induced or enhanced release of IL-1. Monoclonal antibody infusions were less effective (Ignatowski et al., 1999).

Reduced NE Release from Hippocampal Slices Following CCI

Both the LC and the hippocampus have been associated with pain perception and processing (Zhang et al., 1997, Khanna, 1997), and a number of observations support the role of the central noradrenergic nervous system in pain perception. The LC is the largest noradrenergic nucleus in the brain (Swanson and Hartman, 1975), and the noradrenergic neuronal cell bodies in this area represent the primary source of NE in the CNS. NE is also a principal neurotransmitter associated with the modulation of nociception and analgesia (Proudfit, 1998). The hippocampus is a region rich in noradrenergic nerve terminals and receives its NE innervation exclusively from the LC axons projecting their nerve terminals into the hippocampus (Grant and Redmond, 1981, Khanna and Sinclair, 1989). Electrical field stimulation of the hippocampus significantly increases latency in the tail-flick test (Prado and Roberts, 1985), and electrical stimulation of the LC has been demonstrated to produce analgesia (Margalit and Segal, 1979).

α₂-adrenergic autoreceptors, located at noradrenergic axon terminals in the brain, appear to be the main regulators of NE release into the synaptic cleft. Stimulation of these receptors by NE mediates a feedback inhibition of NE release from noradrenergic terminals (Dixon et al., 1979). TNF-α has also been shown to regulate NE release in the rat isolated median eminence, an area with no α₂-adrenergic receptors (Elenkov et al., 1992), in cultured sympathetic neurons (Soliven and Albert, 1992) and in isolated hippocampal slices (Ignatowski and Spengler, 1994).

The involvement of α₂-adrenergic receptors in the CNS during pain processing can be shown by spinal administration of the α₂-adrenergic agonist Clonidine prior to CCI (Yamamoto and Nozaki-Taguchi, 1996). This study showed a 3 days delay in onset of thermal hyperestesia compared to control groups, suggesting a reduction of sympathetic outflow as a mechanism for the delay in hyperesthesia.

When the effect of CCI on NE release was examined by electrical stimulation of hippocampal slices, a significant decrease in NE release was revealed (Ignatowski et al., 1999, Covey et al., 2000). The slices were obtained 8 days post-CCI, and stimulated at both 1 and 4 Hz. The finding that NE release was significantly reduced both at 1 and 4 hz during persistent pain supports the theory of an increased α₂-adrenergic autoreceptor mediated inhibition of NE release during chronic pain. The electrical field stimulation of NE release is frequency dependent, and higher frequencies were expected to further increase NE release due to a higher expression of NE promoting regulatory mechanisms. This observation was further supported by the effect of CCI on the idazoxan (an α₂-adrenergic receptor antagonist) concentration-effect curve. At 8 days post-CCI, the curve was shifted upwards, showing a significantly increased potentiation of NE release. Similar results of reduced NE release could be shown by i.c.v. infusion of rr TNF-α. The changes of NE release were seen at the same time as development of hyperalgesia, and at the same time as raised TNF-α levels could be detected in the hippocampus. This study shows that both during chronic pain and microinfusion of TNF-α into the brain, noradrenergic activity is altered resulting in a decreased NE release in the brain (Covey et al., 2000).

Another interesting feature was the effect of CCI on the TNF-α concentration-effect curves. Even though a significant increase in the inhibition of NE release could be observed 8 days post-CCI, the effect during blockade of the α₂-adrenergic receptor by idazoxan only showed inhibition of NE release similar to controls. This observation suggests an interactive adaption between presynaptic sensitivity to TNF-α and the α₂-adrenergic autoreceptor. The presynaptic sensitivity to TNF-α being negatively conditioned in response to an increased inhibitory effect of α₂-adrenergic autoreceptors, and the net increase of TNF-α mediated inhibition being a result of increased α₂-adrenergic autoreceptor inhibition (Covey et al., 2000).

An interactive relationship between α₂-adrenergic receptor response and TNF-α

Covey and colleagues (Covey et al., 2000) hypothesize that the increase in TNF-α production which occurs in the brain, following peripheral changes after nerve injury, causes a remodelling of α₂-adrenergic receptor regulation of NE release in the CNS, leading to a decrease in NE release that ultimately contributes to the development of central sensitisation and neuropathic pain. The decreased activation of the α₂-adrenergic receptor, due to a decreased NE release, during neuropathic pain would culminate in further increases in TNF-α levels, thereby creating a self-re-inforcing cycle maintaining neuropathic pain states. This theory is strongly supported both by the studies outlined above and by the following studies below.

In electrically stimulated hippocampal slices, TNF-α inhibited NE release in a concentration dependent manner. This effect was potentiated by addition of the α₂-adrenergic antagonist idazoxan. In the absence of exogenous TNF-α, the NE release was significantly increased by idazoxan (Ignatowski and Spengler, 1994). This could imply a compensatory role of TNF-α, enhancing the inhibition of NE release when α₂-adrenergic autoreceptors are blocked, involving α₂-adrenergic receptors in the noradrenergic responsiveness to TNF-α.

Clonidine, an α₂-adrenergic receptor agonist, could significantly decrease electrical stimulated NE release in hippocampal slices (Ignatowski et al., 1996). Further assessment of α₂-adrenergic receptor responsiveness following 1 day of clonidine administration showed a significant decrease in the fractional release of NE, when idazoxan was added to electrical stimulated hippocampal slices, compared to controls with only idazoxan. Assayed TNF-α levels in hippocampus, following 1 day clonidine administration, showed a significant decrease, whereas TNF-α levels in the LC showed an increase as compared to controls (Ignatowski et al., 1996).

The TFN-α inhibition of NE release in electrical stimulated hippocampal slices after 1 day of clonidine administration showed a complete reverse of the concentration-effect curve. Instead of inhibition, TFN-α potentiated NE release. This potentiation could be reversed by addition of idazoxan, but still with less inhibition than that observed for control slices. This shows that α₂-adrenergic receptors are not only involved in the response to TNF-α, but they are also involved in the change of TFN-α responsiveness following acute clonidine administration. This switch from inhibition to potentiation is interesting because clonidine is known to have an acute additive effect with antidepressants, and the switch could represent a clinically important mechanism for the actions of these drugs (Ignatowski et al., 1996). However, the effect is transient. By 14 days of clonidine administration, the TNF-α concentration-effect curve was not shifted towards potentiation of NE release, even though a decrease in the sensitivity of NE inhibition was observed. The addition of idazoxan to the TNF-α concentration-effect curve following 14 days of clonidine administration showed an inhibitory effect on NE release similar to controls treated with idazoxan. This suggests that the α₂-adrenergic autoreceptor is fully functional following chronic clonidine administration, and that the observed reduction in function, following 1 day of clonidine administration, is an acute and transient subsensitization. Bioactive TNF-α levels assayed in the LC and in the hippocampus were significantly decreased in both areas following chronic clonidine administration (Ignatowski et al., 1996).

State of the Art, Conclusion

In conclusion, active immunization and the concept of vaccination against TNF-α in order to provide a treatment of alleviation of symptoms in patients is thus well-documented and the use of anti-TNF-α vaccination has also been demonstrated as an effective means for combating various chronic inflammatory diseases. Furthermore, it is today not possible to effectively treat a wide variety of pain conditions that are discussed above.

### OBJECT OF THE INVENTION

It is an object of the invention to provide a novel approach to the treatment of various pain conditions, i.a. all those pain conditions and pain related symptoms that are described above. It is a further object of the invention to provide useful molecules and pharmaceutical compositions for this particular purpose.

### SUMMARY OF THE INVENTION

A series of experiments performed in rats have demonstrated that vaccination against TNF-α is an effective means of reducing pain. The molecule utilised was a variant of a murine TNF-α monomer wherein is introduced a strong foreign T_{H} epitope that can drive the immune response in rats. It was demonstrated that this molecule induced a strong anti-TNF-α antibody response in the vaccinated rats, and it was demonstrated that this vaccination was effective in reducing pain symptoms in the vaccinated rats when compared to a placebo. Notably, the anti-TNF-α vaccination protected against TNF-α induced pain in the CNS in one animal model, thus demonstrating that peripheral vaccination against TNF-α gave rise to therapeutically effective antibodies in the CNS of the vaccinated animals. This is especially surprising in view of the fact that peripheral administration of a known anti-TNF-α antibody could not be shown to induce any significant effects on the pain perception in the same model.

Hence, the present invention provides for a completely novel approach in the treatment of pain conditions, an approach that entails active vaccination against TFN-α.

Therefore, in its broadest scope the present invention relates to a method for reducing pain or increasing the threshold for nociception in an individual in need thereof, the method comprising administering an effective amount of an agent capable of inducing an active immune response that targets said indivual's autologous tumour necrosis α (TNFα). The pain in question can be any of the pain conditions or related symptoms that are detailed above in the “Background of the Invention” section.

### DETAILED DESCRIPTION OF THE INVENTION

Definitions

In the following, a number of terms used in the present specification and claims will be defined and explained in detail in order to clarify the metes and bounds of the invention.

The terms “T-lymphocyte” and “T-cell” will be used inter-changeably for lymphocytes of thymic origin that are responsible for various cell mediated immune responses as well as for helper activity in the humeral immune response. Likewise, the terms “B-lymphocyte” and “B-cell” will be used inter-changeably for antibody-producing lymphocytes.

A “TNF-α polypeptide” (a “TFN-α monomer”) is herein intended to denote polypeptides having the amino acid sequence of tumour necrosis a monomeric units derived from humans as well as other mammals—TNF-α is a homodimer or a homotrimer. Both unglycosylated forms of TNF-α that are prepared in prokaryotic systems (or that are natively unglycosylated such as human TNF-α) are included within the boundaries of the term as are forms having varying glycosylation patterns (e.g. murine TNF-α is glycosylated in vivo) due to the use of e.g. yeasts or other non-mammalian eukaryotic expression systems. It should, however, be noted that when using the term “a TNF-α polypeptide” it is intended that the polypeptide in question is normally non-immunogenic when presented to the animal to be treated. In other words, the TNF-α polypeptide is a self-protein or is a xeno-analogue of such a self-protein which will not normally give rise to an immune response against TNF-α of the animal in question.

“A substantial fragment” of TNF-α is intended to mean a part of monomeric TNF-α that constitutes at least enough of the amino acid sequence of TNF-α so as to form a domain that folds up in substantially the same 3D conformation as can be found in the native TNF-α.

A “TNF-α variant” is an TNF-α polypeptide which has been either subjected to changes in its primary structure and/or that is associated with elements from other molecular species. Such a change can e.g. be in the form of fusion of an TNF-α polypeptide to a suitable fusion partner (i.e. a change in primary structure exclusively involving C- and/or N-terminal additions of amino acid residues) and/or it can be in the form of insertions and/or deletions and/or substitutions in the TNF-α polypeptide's amino acid sequence. Also encompassed by the term are derivatized TFN-α molecules, cf. the discussion below of modifications of TNF-α.

“An immunogenic TFN-α variant” is herein meant to designate a TNF-α variant that includes substantial parts of the sequence information found in monomeric TNF-α while at the same time being immunogenic in the host where the TNF-α is an autologous protein. That is, such a variant includes at least one foreign T_{H} epitope that is capable of driving an immune response that cross-reacts with the self-TNF-α.

When using the abbreviation “TNF-α” herein, this is intended as a reference to the amino acid sequence of mature, wildtype TNF-α (also denoted “TNFm” and “TNFwt” herein). Mature human TNF-α is denoted hTNF-α, hTNF-αm or hTNF-αwt, and murine mature hTNF-α is denoted mTNF-α, mTNF-αm, or mTNF-αwt. In cases where a DNA construct includes information encoding a leader sequence or other material, this will normally be clear from the context.

The term “polypeptide” is in the present context intended to mean both short peptides of from 2 to 10 amino acid residues, oligopeptides of from 11 to 100 amino acid residues, and poly-peptides of more than 100 amino acid residues. Furthermore, the term also include monomeric proteins, i.e. functional biomolecules consisting of one single polypeptide (whereas the broader term “protein also encompasses proteins comprising at least two polypeptide chains; when proteins comprise at least two polypeptides these are in the present context termed “multimers” or a term is used that indicates the number of monomeric units. Multimers may form complexes, be covalently linked, or may be non-covalently linked. Multimers may be consitituted of identical or non-identical single polypeptides—such multimers are term homomultimers or heteromultimers, respectively. The polypeptide(s) in a protein can be glycosylated and/or lipidated and/or comprise prosthetic groups.

The term “subsequence” means any consecutive stretch of at least 3 amino acids or, when relevant, of at least 3 nucleotides, derived directly from a naturally occurring TNFα amino acid sequence or nucleic acid sequence, respectively.

The term “animal” is in the present context in general intended to denote an animal species (preferably mammalian), such as Homo sapiens, Canis domesticus, etc. and not just one single animal. However, the term also denotes a population of such an animal species, since it is important that the individuals immunized according to the method of the invention all harbour substantially the same TNF-α allowing for immunization of the animals with the same immunogen(s). If, for instance, genetic variants of TFN-α exists in different human population it may be necessary to use different immunogens in these different populations in order to be able to break the autotolerance towards TNF-α in each population. It will be clear to the skilled person that an animal in the present context is a living being which has an immune system. It is preferred that the animal is a vertebrate, such as a mammal.

By the term “down-regulation” is herein meant reduction in the living organism of the biological activity of the TNF-α (e.g. by interference with the interaction between TNF-α and biologically important binding partners for this molecule). The down-regulation can be obtained by means of several mechanisms: Of these, simple interference with the active site in TNF-α by antibody binding is the most simple (and, the focus of WO 98/46642) and also the most important. However, it is also within the scope of the present invention that the antibody binding results in removal of the multimeric protein by scavenger cells (such as macrophages and other phagocytic cells)—the latter is, however, not believed to be an effective mechanism for down-regulation of TFN-α activity, since the serum concentration of TNF-α is extremely minute, whereas the local concentration at the site of action can be quite considerable.

The expression “effecting presentation to the immune system” (and similar terms such as “engaging the immune system”) is intended to denote that the animal's immune system is subjected to an immunogenic challenge in a controlled manner. As will appear from the disclosure below, such challenge of the immune system can be effected in a number of ways of which the most important are vaccination with polypeptide containing “pharmaccines” (i.e. a vaccine which is administered to treat or ameliorate ongoing disease) or nucleic acid “pharmaccine” vaccination. The important result to achieve is that immune competent cells in the animal are confronted with the antigen in an immunologically effective manner, whereas the precise mode of achieving this result is of less importance to the inventive idea underlying the present invention.

The term “immunogenically effective amount” has its usual meaning in the art, i.e. an amount of an immunogen which is capable of inducing an immune response which significantly engages pathogenic agents which share immunological features with the immunogen.

When using the expression that the TFN-α has been “modified” is herein meant a chemical modification of the polypeptide which constitutes the backbone of TNF-α. Such a modification can e.g. be derivatization (e.g. alkylation, acylation, esterification etc.) of certain amino acid residues in the TNF-α sequence, but as will be appreciated from the disclosure below, the preferred modifications comprise changes of (or additions to) the primary structure of the TNF-α amino acid sequence.

When discussing “autotolerance towards TFN-α it is understood that since TNF-α is a self-protein in the population to be vaccinated, normal individuals in the population do not mount an immune response against it; it cannot be excluded, though, that occasional individuals in an animal population might be able to produce antibodies against the native TNF-α, e.g. as part of an autoimmune disorder. At any rate, an animal species will normally only be autotolerant towards its own TNF-α, but it cannot be excluded that analogues derived from other animal species or from a population having a different phenotype would also be tolerated by said animal.

A “foreign T-cell epitope” (or: “foreign T-lymphocyte epitope”) is a peptide which is able to bind to an MHC molecule and which stimulates T-cells in an animal species—an alternate term is therefore. Preferred foreign T-cell epitopes in the invention are “promiscuous” (or “universal” or “broad-range”) epitopes, i.e. epitopes which bind to a substantial fraction of a particular class of MHC molecules in an animal species or population. Only a very limited number of such promiscuous T-cell epitopes are known, and they will be discussed in detail below. It should be noted that in order for the immunogens which are used according to the present invention to be effective in as large a fraction of an animal population as possible, it may be necessary to 1) insert several foreign T-cell epitopes in the same TNF-α variant or 2) prepare several TNF-α variants wherein each variant has a different promiscuous epitope inserted. It should be noted also that the concept of foreign T-cell epitopes also encompasses use of cryptic T-cell epitopes, i.e. epitopes which are derived from a self-protein and which only exerts immunogenic behaviour when existing in isolated form without being part of the self-protein in question—the latter approach is, however, relatively dangerous, cf. the discussion below that focuses on the dangers relating to accidental induction of uncontrollable autoimmunity.

A “foreign T helper lymphocyte epitope” (a foreign T_{H} epitope) is a foreign T cell epitope which binds an MHC Class II molecule and can be presented on the surface of an antigen presenting cell (APC) bound to the MHC Class II molecule. In the present specification and claims, a T_{H} epitope may also be denoted “an MHC Class II molecule binding peptide or amino acid sequence”.

An “MHC Class II binding amino acid sequence that is heterologous to TNF-α” is therefore an MHC Class II binding peptide that does not exist in TNF-α. Such a peptide will, if it is also truly foreign to the animal species harbouring TNF-α protein, be a foreign T_{H} epitope.

A “functional part” of a (bio)molecule is in the present context intended to mean the part of the molecule which is responsible for at least one of the biochemical or physiological effects exerted by the molecule. It is well-known in the art that TNF-α and other effector molecules have an active site which is responsible for the effects exerted by the molecule in question. Other parts of the molecule may serve a stabilizing or solubility enhancing purpose and can therefore be left out if these purposes are not of relevance in the context of a certain embodiment of the present invention. However, according to the present invention, it is preferred to utilise as much of the TNF-α as possible—this is because WO 98/46642 has demonstrated that only it is possible to induce neutralizing antibodies if one avoids specific regions in the TNF-α monomer.

The term “adjuvant” has its usual meaning in the art of vaccine technology, i.e. a substance or a composition of matter which is 1) not in itself capable of mounting a specific immune response against the immunogen of the vaccine, but which is 2) nevertheless capable of enhancing the immune response against the immunogen. Or, in other words, vaccination with the adjuvant alone does not provide an immune response against the immunogen, vaccination with the immunogen may or may not give rise to an immune response against the immunogen, but the combination of vaccination with immunogen and adjuvant induces an immune response against the immunogen which is stronger than that induced by the immunogen alone.

“Targeting” of a molecule is in the present context intended to denote the situation where a molecule upon introduction in the animal will appear preferentially in certain tissue(s) or will be preferentially associated with certain cells or cell types. The effect can be accomplished in a number of ways including formulation of the molecule in composition facilitating targeting or by introduction in the molecule of groups which facilitate targeting. These issues will be discussed in detail below.

“Stimulation of the immune system” means that a substance or composition of matter exhibits a general, non-specific immunostimulatory effect. A number of adjuvants and putative adjuvants (such as certain cytokines) share the ability to stimulate the immune system. The result of using an immunostimulating agent is an increased “alertness” of the immune system meaning that simultaneous or subsequent immunization with an immunogen induces a significantly more effective immune response compared to isolated use of the immunogen.

Preferred Embodiments

The general concept of the present invention is to prevent, treat or alleviate pain by actively inducing immunity against TNF-α. Hence, any agent that is capable of inducing a specific immune response against autologous TNF-α is in principle useful as an immunogenic agent in the present invention.

It is therefore within the scope of the present invention to utilise any agents that will provide for induction of active immunity against self-TNF-α in an individual and in essence such an agent can be an immunogenic TNF-α variant, a nucleic acid fragment encoding an immunogenic TNF-α variant, and a non-pathogenic bacterium or virus that harbours an a nucleic acid fragment encoding an immunogenic TFN-α variant.

Therefore, even though the most preferred mode of the invention entails vaccinating with a protein vaccine that is prepared according to the principles set forth in DK PA 2001 01702 and in the corresponding international patent application PCT/DK02/00764 that claims the priority thereof, the precise implementation of how to break tolerance against TNF-α is not crucial as such—in order to alleviate pain, it is, according to the present invention, crucial to induce an effective immune response against autologous TNF-α.

In general, most self-proteins such as TNF-α are tolerated by the mature immune system, but there are various ways of breaking this self-tolerance.

One way is to simply admix TNF-α or an immunologically effective fragment thereof with a very strong adjuvant (cf. discussion of adjuvants below). However, since the immune response is driven by T_{H} cells that recognize T_{H} epitopes in the vaccine molecule, there is a high risk that the immune response that would ensue from such a strategy would be driven by T_{H} cells that recognise T_{H} epitopes found in the autologous TNF-α. This can in turn result in an uncontrolled autoimmune response that does not terminate simply because immunisations are stopped, and therefore this particular mode of the invention is not regarded as a preferred embodiment. Even more problematic is the fact that TNF-α has highly toxic effects, and therefore the administration of immunogenically effective amounts of non-detoxified TNF-α would most likely put the vaccinated individual's life at risk (unless a formulation is used that ensures that the vaccine composition is kept locally in the tissue of introduction).

Hence, the method of the invention is preferably put into practice by engaging the immune system with variants of TFN-α that are 1) non-toxic and/or 2) that include at least one foreign T_{H} epitope that can drive the immune response. Both these features provides for a safe vaccine product, i.e. feature 1 reduces the risk of acute toxic effects and feature 2 reduces/eliminates the risk of inducing an uncontrollable autoimmune condition (previous work with applicant's immunisation technology has repeatedly shown that when using a strong foreign T_{H} epitope as part of the immunogen, the immune response induced can be “turned off” again by simply discontinuing the immunization scheme).

The following discussion will therefore focus on how autologous TNF-α can be modified so as to, when engaging the immune system, induce antibodies cross-reactive with native TNF-α in the vaccinated individual. After that, the discussions will focus on the means by which TNF-α variants are brought to engage the immune system, and finally a discussion of various useful production tools is given.

Modification of TNF-α

Known methods for breaking self-tolerance entail traditional hapten carrier conjugate technology, fusion protein technology as well as approaches where single T_{H} epitopes are utilised as part of the immunogen. What is common for all these known approaches is the provision of T_{H} epitopes that will be recognized by the vaccinated individual.

Traditional chemical coupling of a large carrier molecule (cf. the discussion of such carriers below and the utilisation thereof) to TFN-α or a fragment thereof is therefore a possibility—the TNF-α may in this case both be a monomer TNF-α subunit or di- or trimeric TFN-α molecules.

A more recent version of the traditional conjugate carrier technology is preparation of fusion constructs where one fusion partner would be TFN-α or a fragment thereof and where the other fusion partner would be any suitable carrier polypeptide (tetanus toxoid, diphtheria toxoid, KLH etc. cf. below).

Finally, applicant's own technology for breaking of self-tolerance can in general be described as a “minimum disturbance” approach, where the T cell help is induced by including (the minimal) necessary T_{H} epitopic elements in the immunogen while at the same time aiming at preserving as many B-cell epitopes as at all possible. This approach is the focus of WO 95/05849 and WO 98/46642 (which especially focuses on preparation of vaccine agents capable of inducing neutralizing anti-TNFα antibodies); a nucleic acid vaccination version of this type of technology is also described in applicant's own WO 00/20027. In brief, applicant's technology entails introduction of one single or very few foreign, promiscuous T_{H} epitopes in the amino acid sequence of the vaccine antigen—the introduction is made in regions of the antigen that provide the least degree of disturbance of the 3D structure of the molecule.

Moreover, it is of course also possible to identify single interesting B-cell epitopes in TNF-α and couple these (by means of fusion or traditional chemical coupling methods) to single epitopic peptides or to larger protein carriers.

The present applicant is also the assignee of an international patent application that relates to a different approach, where coupling of a mixture of peptides (self-derived as well as foreign peptides, e.g. T_{H} epitopes) are coupled to an activated polyhydroxypolymer (e.g. tresylated dextran). The conjugates thus obtained are also interesting embodiments for use in the present methods of pain-treatment. This particular technology is the subject matter of WO 02/066056.

In general, the disclosures of WO 95/05849, WO 98/46642, WO 00/20027, DK PA 2001 01702 (and the corresponding, priority claiming PCT/DK02/00764) and WO 02/066056, insofar as these relate to generally applicable technology or insofar as these relate to immunization against autologous TNF-α (especially those technologies that relate to induction of neutralising anti-TNF-α antibodies), are all incorporated by reference in this document. That is, all teachings that can be found concerning immunization against self-proteins in general and against TNF-α in particular are highly relevant with a view to work the present invention and all these technologies and specifically disclosed vaccine variants are considered to be highly interesting and important embodiments of the present invention.

Preferred TNF-α Based Constructs

It is advantageous if the immunogenic variant useful in the invention displays, a substantial fraction of B-cell epitopes found in self-TNF-α. A substantial fraction of B-cell epitopes is herein intended to mean a fraction of B-cell epitopes that antigenically characterizes TNF-α versus other proteins. It is preferred that the substantial fragments displays essentially all B-cell epitopes found native TFN-α—of course and in accordance with the principles set forth herein, introduction of minor changes in the monomer sequence may be necessary. For instance an amino acid sequence derived from TNF-α may be modified by means of amino acid insertion, substitution, deletion or addition so as to reduce toxicity of the variant as compared to the native TNF-α and/or so as to introduce the MHC Class II binding amino acid sequence, if it is undesired to have that sequence positioned in a fusion partner or a conjugation partner.

An especially preferred embodiment provides for the use of an immunogenic TNF-α variant, wherein each of the substantial fractions comprises essentially the complete amino acid sequence of each monomeric TNF-α unit, either as a continuous sequence or as a sequence including inserts. That is, only insignificant parts of the monomeric unit's sequence are left out of the variant, e.g. in cases where such a sequence does not contribute to tertiary structure of the monomeric unit or quaternary structure of the multimeric protein. However, this embodiment allows for substitution or insertion of the monomer, as long as the 3D structure of the multimeric TFN-α protein is maintained. Hence, it is especially advantageous if the immunogenic variant is one, wherein amino acid sequences of all monomeric TNF-α units are represented in the variant, and it is particularly advantageous if the variant includes the complete amino acid sequences of (all) the TNF-α monomers constituting the TFN-α dimer or trimer, either as unbroken sequences or as sequences including inserts.

As will appear, it is therefore preferred that the 3-dimensional structure of the complete TNF-α is essentially preserved in the variant.

Maintenance of a substantial fraction of B-cell epitopes or even the 3-dimensional structure of TNF-α that is subjected to modification as described herein can be achieved in several ways. One is simply to prepare a polyclonal antiserum directed against monomeric, dimeric or trimeric TNF-α (e.g. an antiserum prepared in a rabbit) and thereafter use this antiserum as a test reagent (e.g. in a competitive ELISA) against the variants which are produced. Modified versions (variants) which react to the same extent with the antiserum as does TNF-α must be regarded as having the same 3D structure as the TNF-α molecule whereas variants exhibiting a limited (but still significant and specific) reactivity with such an antiserum are regarded as having maintained a substantial fraction of the original B-cell epitopes.

Alternatively, a selection of monoclonal antibodies reactive with distinct epitopes on the monomeric, dimeric or trimeric TNF-α can be prepared and used as a test panel. This approach has the advantage of allowing 1) an epitope mapping of TNF-α and 2) a mapping of the epitopes which are maintained in the variants prepared.

Of course, a third approach would be to resolve the 3-dimensional structure of TNF-α (cf. above) and compare this to the resolved three-dimensional structure of the variants prepared. Three-dimensional structure can be resolved by the aid of X-ray diffraction studies and NMR-spectroscopy. Further information relating to the 3D structure can to some extent be obtained from circular dichroism studies which have the advantage of merely requiring the polypeptide in pure form (whereas X-ray diffraction requires the provision of crystallized polypeptide and NMR requires the provision of isotopic variants of the polypeptide) in order to provide useful information about the 3D structure of a given molecule. However, ultimately X-ray diffraction and/or NMR are necessary to obtain conclusive data since circular dichroism can only provide indirect evidence of correct 3-dimensional structure via information of secondary structure elements.

The immunogenic TNF-α variant used in the invention may include a peptide linker that includes or contributes to the presence in the variant of at least one MHC Class II binding amino acid sequence that is heterologous to the TFN-α protein. This is particularly useful in those cases where it is undesired to alter the amino acid sequence corresponding to monomeric units in TNF-α. Alternatively, the peptide linker may be free of and not contributing to the presence of an MHC Class II binding amino acid sequence in the animal species from where the TNF-α protein is derived; this can conveniently be done in cases where it is necessary to utilise a very short linker or where it is advantageous to e.g. detoxify a potentially toxic TFN-α variant by introducing the MHC Class II binding element in an active site.

It is preferred that the MHC Class II binding amino acid sequence binds a majority of MHC Class II molecules from the animal species from where the multimeric protein has been derived, i.e. that the MHC Class II binding amino acid sequence is universal or promiscuous.

It is of course important that this sequence serves its purpose as a T cell epitope in the species for which the immunogen is intended to serve as a vaccine constituent. There exists a number of naturally occurring “promiscuous” T-cell epitopes which are active in a large proportion of individuals of an animal species or an animal population and these are preferably introduced in the vaccine, thereby reducing the need for a very large number of different variants in the same vaccine. Hence, the at least one MHC Class II binding amino acid sequence is preferably selected from a natural T-cell epitope and an artificial MHC-II binding peptide sequence. Especially preferred sequences are a natural T-cell epitope is selected from a Tetanus toxoid epitope such as P2 (SEQ ID NO: 2) or P30 (SEQ ID NO: 3), a diphtheria toxoid epitope, an influenza virus hemagluttinin epitope, and a P. falciparum CS epitope.

Over the years a number of other promiscuous T-cell epitopes have been identified. Especially peptides capable of binding a large proportion of HLA-DR molecules encoded by the different HLA-DR alleles have been identified and these are all possible T-cell epitopes to be introduced in the TFN-α variants used according to the present invention. Cf. also the epitopes discussed in the following references which are hereby all incorporated by reference herein: WO 98/23635 (Frazer I H et al., assigned to The University of Queensland); Southwood S et. al, 1998, J. Immunol. 160: 3363-3373; Sinigaglia F et al., 1988, Nature 336: 778-780; Chicz R M et al., 1993, J. Exp. Med 178: 27-47; Hammer J et al., 1993, Cell 74: 197-203; and Falk K et al., 1994, Immunogenetics 39: 230-242. The latter reference also deals with HLA-DQ and -DP ligands. All epitopes listed in these 5 references are relevant as candidate natural epitopes to be used in TFN-α variants employed in the present invention, as are epitopes that share common motifs with these.

Alternatively, the epitope can be any artificial T-cell epitope which is capable of binding a large proportion of MHC Class II molecules. In this context the pan DR epitope peptides (“PADRE”) described in WO 95/07707 and in the corresponding paper Alexander J et al., 1994, Immunity 1: 751-761 (both disclosures are incorporated by reference herein) are interesting candidates for epitopes to be used according to the present invention. It should be noted that the most effective PADRE peptides disclosed in these papers carry D-amino acids in the C- and N-termini in order to improve stability when administered. However, the present invention primarily aims at incorporating the relevant epitopes as part of the variant which should then subsequently be broken down enzymatically inside the lysosomal compartment of APCs to allow subsequent presentation in the context of an MHC-II molecule, and therefore it is not expedient to incorporate D-amino acids in the epitopes used in the present invention.

One especially preferred PADRE peptide is the one having the amino acid sequence AKFVAAWTLKAAA (SEQ ID NO: 4) or an immunologically effective subsequence thereof. This, and other epitopes having the same lack of MHC restriction are preferred T-cell epitopes, which should be present in the variants used in the inventive method. Such super-promiscuous epitopes will allow for the simplest embodiments of the invention wherein only one single TNF-α variant is presented to the vaccinated animal's immune system.

Preferred embodiments of the invention include modification by introducing at least one foreign immunodominant T_{H} epitope. It will be understood that the question of immune dominance of a T_{H} epitope depends on the animal species in question. As used herein, the term “immunodominance” simply refers to epitopes which in the vaccinated individual gives rise to a significant immune response, but it is a well-known fact that a T_{H} epitope which is immunodominant in one individual is not necessarily immunodominant in another individual of the same species, even though it may be capable of binding MHC-II molecules in the latter individual.

As mentioned above, the introduction of a foreign T-cell epitope can be accomplished by introduction of at least one amino acid insertion, addition, deletion, or substitution. Of course, the normal situation will be the introduction of more than one change in the amino acid sequence (e.g. insertion of or substitution by a complete T-cell epitope) but the important goal to reach is that the variant, when processed by an antigen presenting cell (APC), will give rise to such a T-cell epitope being presented in context of an MCH Class II molecule on the surface of the APC. Thus, if the amino acid sequence of the monomeric TNF-α unit in appropriate positions comprises a number of amino acid residues which can also be found in a foreign T_{H} epitope then the introduction of a foreign T_{H} epitope can be accomplished by providing the remaining amino acids of the foreign epitope by means of amino acid insertion, addition, deletion and substitution. In other words, it is not necessary to introduce a complete T_{H} epitope by insertion or substitution.

According to the present invention, the TFN-α variant may also form part of larger molecule wherein it is coupled to at least one functional moiety, the presence of which does not interfer negatively to a significant degree with the antibody-accessability of the variant. The nature of such moieties (which may be fused to the TNF-α variant) are set forth in the claims and is discussed in detail for these moieties in WO 00/65058 which is hereby incorporated by reference herein.

It is preferred that the number of amino acid insertions, deletions, substitutions or additions is at least 2, such as 3, 4, 5, 6, 7, 8, 9, 10, 11, 12, 13, 14, 15, 16, 17, 18, 19, 20, 21, and 25 insertions, substitutions, additions or deletions. It is furthermore preferred that the number of amino acid insertions, substitutions, additions or deletions is not in excess of 150, such as at most 100, at most 90, at most 80, and at most 70. It is especially preferred that the number of substitutions, insertions, deletions, or additions does not exceed 60, and in particular the number should not exceed 50 or even 40. Most preferred is a number of not more than 30. With respect to amino acid additions, it should be noted that these, when the resulting construct is in the form of a fusion polypeptide, is often considerably higher than 150.

With respect to the issues of particular considerations concerning immunodominance and MHC restriction of T helper epitopes, reference is made to WO 00/65058 where a thorough description can be found.

According to the present invention, especially preferred TNF-α variants are those human TNF-α variants that are disclosed in WO 98/46642 (optionally variants where the P2 and P30 epitope inserts are exchanged with other promiscuous epitopes, cf. the above discussion of these).

In nature, human TNF-α (SEQ ID NO: 1 shows the monomer polypeptide's amino acid sequence) exists as both a dimer and a trimer. In applicant's Danish priority application DK PA 2001 01702 (and in the subsequent priority claiming international patent application PCT/DK02/00764) is disclosed a novel approach for providing useful immunogenic versions of autologous multimeric proteins and a number of variants of TNF-α are described in detail. All of these variants are particularly preferred embodiments of the present invention.

In the following is provided a summary of those types of TNF-α variants that are the subject of PCT/DK02/00764 and therefore are used in the most preferred embodiments of the present invention:

One set of useful TNF-α constructs comprises

1) two or three complete TFN-α monomers joined end-to-end by a peptide linker, wherein at least one peptide linker includes at least one MHC Class II binding amino acid sequence, and 2) two or three complete TNF-α monomers joined end-to-end by an inert peptide linker, wherein at least one of the monomers includes at least one foreign MHC Class II binding amino acid sequence or wherein at least one foreign MHC Class II binding amino acids sequence is fused to the N- or C-terminal monomer, optionally via an inert linker.

Particularly interesting are immunogenic TNFα molecules with high stability, since monomeric TNFα constructs tend to be relatively unstable, cf., however, the discussion below.

A gene encoding the 3 TNFα subunits linked together by epitopes and/or inert peptide linkers has been produced, cf. PCT/DK02/00764. The goal has been to generate variant TNF-α molecules with a conformation as close to the native TFN-α trimer as possible. The variants have been designed to efficiently elicit neutralizing antibodies against wtTNF-α. The most suitable TNFα variants are soluble and stable proteins in the absence of detergents or other kinds of additives that could disrupt the protein conformation.

By expressing the three monomers linked together as one single polypeptide chain using linkers and T_{H} epitopes, it is intended to prepare TNFα variants that are more stable than previous variant TNFα immunogens. This will allow preservation of the TNFα structure, by introduction of the necessary T_{H} epitopes outside of stabilizing hydrogen bonds, salt bridges or disulfide bridges.

From the X-ray crystal structure of TNFα it is seen that the first 5 residues of the N terminal are too flexible to allow a structure determination. The C-terminus, however, is located close to the middle of the monomer interface and is less flexible. The distance between the C alpha atoms of Arg-6 and Leu-157 is 10 Å, which is the distance of 3-4 amino acid residues. Therefore it seems to be possible to link the monomeric subunits directly together, but since the C-terminals are located at a delicate site, it will probably be advantageous to use flexible linkers, e.g. glycine linkers, for this connection.

Five variants have until now been designed utilising this “monomerized trimer” approach. The control TNF_T0(TNFα Trimer number 0, SEQ ID NO: 22 in PCT/DK02/00764) consists of the three monomers directly linked together by 2 separate glycine linkers (GlyGlyGly). TNF_T0 is designed so as to be as stable as the wild type trimeric protein. Of course, other inert flexible linkers known in the art of protein chemistry may be used instead of the above-mentioned glycine linkers, the important feature being that the flexible linker does not interfer adversely with the monomerized protein's capability of folding into a 3D structure that is similar to the 3D structure of physiologically active wtTNFα.

The TNF_T0 construct is expressed as a soluble protein in E. coli, and it has been used to prepare the exemplary construct TNF_T4 (SEQ ID NO: 57 in PCT/DK02/00764), which is a variant wherein the PADRE MHC Class II binding peptide (SEQ ID NO: 4) is introduced. In this construct, the ratio between monomeric units and foreign epitopes are thus 1 epitope per 3 monomers, instead of 1 epitope per monomer as is the case in prior art variants that relied on immunogenized monomeric proteins—this is also the case for SEQ ID NO: 55 in PCT/DK02/00764). This fact provides a potentially positive influence on the trimer stability. An offspring from this approach is the TNF_C2 variant (SEQ ID NO: 28 in PCT/DK02/00764, cf. below), which is a TNFα monomer with a PADRE epitope in the same position as in TNF_T4.

In parallel, the tetanus toxoid P2 and P30 epitopes (SEQ ID NOs: 2 and 3, respectively), have been used in the TNF_T1 and TNF_T2 variants (SEQ ID NOs: 49 and 51 in PCT/DK02/00764, respectively), containing one epitope in each linker region, and also in TNF_T3 (SEQ ID NO: 59 in PCT/DK02/00764) that contains one C-terminal epitope and one in the second linker region. Proteins are mostly folded from the N-terminal toward the C-terminal. The idea underlying TNF_T3 is that when the first two N-terminal domains fold up they will function as internal chaperones for the third domain (monomer), which is enclosed by epitopes.

It has been discovered that in addition to the technology described in detail above, where polymeric proteins are “monomerized”, TNF-α (and possibly many other multimeric proteins) allows for the production of monomers that 1) include at least one stabilising mutation and/or 2) include at least one non-TNF-α derived MHC Class II binding amino acid sequence, where these TNF-α monomer variants are capable of folding correctly into a tertiary structure that subsequently allows for the formation of dimeric and trimeric TNF-α proteins having a correct quarternary structure (as evidenced by these having receptor binding activity). Hence, in these constructs it has been possible to prepare variants of monomeric TNF-α that does not necessarily need to be produced as monomerized trimers because the changes introduced in the monomer sequences introduce so limited disruption of the monomer's tertiary structure that a di- or trimer can be formed. All such variant are expressible as soluble proteins from bacterial cells.

Hence, it is possible to prepare immunogenic TFN-α variants according to the following strategies that can be combined and which may further be combined with the already discussed “monomerization approach” (since these particular modifications all are non-destructive by nature):

The Flexible Loop Strategy of PCT/DK02/00764

Insertion of the PADRE epitope (SEQ ID NO: 4) into loop 3 in position Gly108-Ala109 is a promising approach to prepare TNF-α variants with a structure closely resembling the native TNF-α molecule. It has been deduced from the TNF-α crystal structure that a T_{H} epitope inserted directly into this position will not have any neighbouring amino acid residues in close proximity to interact with. Studies with TNF34 (SEQ ID NO: 18 in PCT/DK02/00764), the first PADRE construct made according to this approach, has shown that approximately 5% of the expressed protein TNF34 was soluble in E. coli and 95% of the TNF34 was expressed as inclusion bodies when the bacterial host cells were grown at 37° C. but after an adaptation of the fermentation process where the fermentation temperature is 25° C., the yields of soluble protein from the fermentation is close to 100%. Hence, optimization of growth conditions increases the yield of soluble protein.

A number of other constructs have been prepared (TNF35-TNF39, SEQ ID NOs, 23, 24, 25, 26, and 27 in PCT/DK02/00764), where all of these solely rely on introduction of SEQ ID NO: 4 into the flexible loop 3.

Stability Enhancing Mutations of PCT/DK02/00764

Introduction of T_{H} epitopes in the flexible loop 3 could potentially destabilize the structure of the TNF-α variant. However, this potential destabilization can be counteracted by stabilization of the structure through introduction of cysteines that will form a disulfide bridge. A cystine pair in two different positions have until now been introduced in variants TNF34-A and TNF34-B (SEQ ID NOs: 29 and 30 in PCT/DK02/00764). Also, the flexible N-terminal (the first 8 amino acids) that is known to reduce the strength of the receptor interaction has be deleted in parallel, hence the variant TNF34-C (SEQ ID NO: 31 in PCT/DK02/00764). The disulfide bridge is introduced in the monomer for stabilization of the epitope insertion site together with the naturally occurring disulfide bridge (Cys-67 Cys-101). This strategy would also stabilise both a TNFα monomer as such and a monomerized di- or trimer.

Other Conststructs of PCT/DK02/00764

Several different strategies have been employed in the design of variants that will be soluble expression products. TNFX1.1-2 (SEQ ID NOs: 32 and 33 in PCT/DK02/00764) are based on insertions of SEQ ID NO: 4 in the first loop of TNF-α, where the insertion site is located at an intron position. In TNFX2.1 (SEQ ID NO: 34 in PCT/DK02/00764) an artificial “stalk” region is created containing an insertion of SEQ ID NO: 4.

Mutations of TNF-α have revealed that large hydrophobic amino acid substitutions, pointing into the trimer interface, stabilize the trimer structure. TNFX3.1 and TNFX3.2 (SEQ ID NOs: 35 and 36 in PCT/DK02/00764) are proposals to stabilize the existing TNF34 variant. TNFX4.1 (SEQ ID NO: 37 in PCT/DK02/00764) uses di-glycine linkers to diminish structural constrains from the PADRE peptide on the overall TNF34 structure. TNFX5.1 (SEQ ID NO: 38 in PCT/DK02/00764) employs, as an insertion point, a loop structure found in the TNF family member BlyS. TNFX6.1-2, TNF7.1-2 and TNFX8.1 (SEQ ID NOs: 39, 40, 41, 42, and 43 in PCT/DK02/00764) are further variants. TNFX9.1 and TNFX9.2 (SEQ ID NOs: 44 and 45 in PCT/DK02/00764) are TNF34 variants that utilize identical overlapping TNFα sequences of 4-6 amino acids both pre and post the epitope. Finally, two variants (SEQ ID NOs: 46 and 47 in PCT/DK02/00764) are P2/P30 double variants in the same location as for the PADRE peptide in TNF34.

Further, from the crystal structure of TNFα it is observed that one stabilizing salt bridge is present within the TNFα monomer between the residues Lys-98 and Glu-116. The definition of a salt-bridge is an electrostatic interaction between side chain oxygens in Asp or Glu and positive charged atom side chain nitrogens in Arg, Lys or His with an interatomic distance less than 7.0 Ångstrom. By site directed substitition mutations of Lys-98 with Arg or His at this position in combination with substitutions of Glu 116 with Asp, an improvement of the stability for this salt bridge and thereby the stability of the trimer molecule could be attained. It is also possible to exchange these salt bridges with disulphide bridges, in a manner described above.

It has been observed that murine TNFα is considerably more stable than the human TNFα regarding to solubility and proteolysis. Improvement of TNFα variants includes making site directed mutants so as to mimic murine TNFα crystal structure to obtain more proteolytically stable TNFα product.

From the x-ray structures of human and murine TNFα it is seen that the centre of the trimer (in the middle of the three TNFα monomers) is held together due to hydrophobic forces, whereas the top and the bottom of the trimer is connected due to natural occurring salt bridges. Therefore, by screening these salt bridges for stronger connections, the stability of the TNFα trimer would also be improved.

Finally, the preliminary results obtained with the TNFα variants of the present invention have surprisingly demonstrated that the variants are physiologically active, at least in the sense that they bind the TNF-receptors. However, since TNFα is a toxic protein, it is desired to prepare safe variants that will not cause severe side effects in subjects immunised with a vaccine according to the invention. Therefore, it is also an important embodiment of the invention to include detoxifying mutations in the constructs if these upon testing in relevant toxicity models are demonstrated to be of potential danger for vaccinated individuals.

A number of point mutations are known in the art to detoxify TNFα or at least reduce toxicity to a large extent. These point mutations will, if necessary, be introduced into the variants of the present invention. Expecially preferred mutations are substitutions corresponding to mature TNFα of Tyr-87 with a Ser, of Asp-143 with Asn, and of Ala-145 with Arg. Further, all effective mutations mentioned in Loetscher, H., Stueber, D., Banner, D., Mackay, F. and Lesslauer, W. 1993 JBC 268 (35) 26350-7, are also interesting embodiments in the detoxifying embodiments of the present invention.

In summary, the following specific TNFα variants have been prepared according to the present invention:

| | Last aa | First aa | Amino acids | | |
|---|---|---|---|---|---|
| TNF Con- | before | after | deleted by | | Total |
| structs | epitope | epitope | insert | Mutations | length |
| TNF34 | 108 | 109 | — | | 170 |
| TNF35 | 106 | 107 | — | | 170 |
| TNF36 | 107 | 108 | — | | 170 |
| TNF37 | 108 | 110 | A | | 169 |
| TNF38 | 108 | 112 | AEA | | 167 |
| TNF39 | 106 | 112 | EGAEA | | 165 |
| TNFC2 | 170 | | — | GGG + PADRE added C- | 173 |
| | | | | terminally | |
| TNF34-A | 108 | 109 | — | Q67C, A111C | 170 |
| TNF34-B | 108 | 109 | — | A96C, I118C | 170 |
| TNF34-C | 108 | 109 | — | N-terminal VRSSSRTP | 162 |
| | | | | are deleted | |
| TNFX1.1 | 17 | 19 | A | | 169 |
| TNFX1.2 | 17 | 96 | ANPQA | | 165 |
| TNFX2.1 | 0 | 2 | V | PADRE added N- | 170 |
| | | | | terminally | |
| TNFX3.1 | 108 | 109 | — | L157F | 170 |
| TNFX3.2 | 108 | 109 | — | V49F | 170 |
| TNFX4.1 | 108 | 109 | — | Two glycines before | 174 |
| | | | | and after PADRE | |
| TNFX5.1 | 83 | 87 | AVS | | 167 |
| TNFX6.1 | 132 | 146 | SAEINRPDYLDFA | | 157 |
| TNFX6.2 | 135 | 146 | INRPDYLDFA | | 160 |
| TNFX7.1 | 63 | 77 | FKGQGCPSTHVLL | | 157 |
| TNFX7.2 | 71 | 85 | THVLLTHTISRIA | | 157 |
| TNFX8.1 | 126 | 140 | EKGDRLSAEINRP | | 157 |
| TNFX9.1 | 108 | 103 | — | The six amino acids | 176 |
| | | | | preceeding PADRE are | |
| | | | | duplicated after the | |
| | | | | epitope | |
| TNFX9.2 | 108 | 105 | — | The four amino acids | 174 |
| | | | | preceeding PADRE are | |
| | | | | duplicated after the | |
| | | | | epitope | |
| TNF34-P2-P30 | 108 | 109 | — | Both P2 and P30 | 194 |
| TNF34-P30-P2 | 108 | 109 | — | Both P30 and P2 | 194 |

The numbers used are from the N-terminal V in SEQ ID NO: 17 in PCT/DK02/00764 (that is, from amino acid no. 2 in SEQ ID NO: 17 in PCT/DK02/00764). Preceding the N-terminal Valine is in some sequences a Methionine used for translation start.

The most preferred protein constructs used in the method of the present invention are thus those represented by any one of SEQ ID NOs: 18, 23, 24, 25, 26, 27, 28, 29, 30, 31, 32, 33, 34, 35, 36, 37, 38, 39, 40, 41, 42, 43, 44, 45, 46, 47, 49, 51, 53, 55, 57, and 59 in PCT/DK02/00764, as well as any amino acid sequence derived therefrom that only include conservative amino acid changes or detoxifying amino acid changes thereof.

At any rate, it is an important embodiment that all of these TNFα variants discussed above are expressible as soluble proteins from bacterial cells such as E. coli.

The preferred vector is pET28b+ when the goal is expression from E. coli, p2Zop2F (SEQ ID NO: 60 in PCT/DK02/00764) is the vector used for insect cell expression, and pHP1 (or its commercially available “twin” pCI) is the vector used for expression in mammalian cells.

Hence, and in view of the above, an important part of the invention relates to the method of the invention where the immunogen is an immunogenic variant of human TNFα, wherein the variant includes at least one foreign MHC Class II binding amino acid sequence and further has the characteristic of being
a human TNFα monomer or a monomerized variant of TNFα, wherein has been inserted or in-substituted at least one foreign MHC Class II binding amino acid sequence into flexible loop 3, and/or
a human TNFα monomer or a monomerized variant of TNFα, wherein has been introduced at least one disulfide bridge that stabilises the TNFα monomer 3D structure, and/or
a human TNFα monomer or a monomerized variant of TNFα, wherein any one of amino acids 1, 2, 3, 4, 5, 6, 7, 8, and 9 in the amino terminus have been deleted, and/or
a human TNFα monomer or a monomerized variant of TNFα, wherein an inserted or in-substituted at least one foreign MHC Class II binding amino acid sequence into loop 1 in an intron position, and/or
a human TNFα monomer or a monomerized variant of TNFα, wherein at least one foreign MHC Class II binding amino acid sequence is introduced as part of an artificial stalk region in the N-terminus of human TNFα, and/or
a human TNFα monomer or a monomerized variant of TNFα, wherein at least one foreign MHC Class II binding amino acid sequence is introduced so as to stabilize the monomer structure by increasing the hydrophobicity of the trimeric interaction interface, and/or
a human TNFα monomer or a monomerized variant of TNFα, wherein at least one foreign MHC Class II binding amino acid sequence flanked by glycine residues is inserted or in-substituted in the TNFα amino acid sequence, and/or
a human TNFα monomer or a monomerized variant of TNFα, wherein at least one foreign MHC Class II binding amino acid sequence is inserted or in-substituted in the D-E loop, and/or
a human TNFα monomer or a monomerized variant of TNFα, wherein at least one foreign MHC Class II binding amino acid sequence is inserted or in-substituted between two identical subsequences of human TNFα, and/or
a human TNFα monomer or a monomerized variant of TNFα, wherein at least one salt bridge in human TNFα has been strengthened or substituted with a disulphide bridge, and/or
a human TNFα monomer or a monomerized variant of TNFα, wherein solubility and/or stability towards proteolysis is enhanced by introducing mutations that mimic murine TNFα crystalline structure, and/or
a human TNFα monomer or a monomerized variant of TNFα, wherein potential toxicity is reduced or abolished by introduction of at least one point mutation.

In general, it has been found that all of the best suited immunogenic variants for use in the invention are those that are soluble proteins already at the stage when they are produced and isolated in soluble form from their recombinant host cells.

Preferred Modes of Presenting Modified TNF-α to the Immune System.

Generally speaking, there are 3 main methodologies for inducing immune responses according to the present invention: Protein/peptide vaccination, DNA vaccination, and live vaccination (i.e. vaccination with genetically modified virus or microorganisms). These 3 general technologies will be discussed in detail in the following:

Protein/Polypeptide Vaccination and Formulation

When effecting presentation of the TNF-α variants to an animal's immune system by means of administration thereof to the animal, the formulation of the polypeptide follows the principles generally acknowledged in the art.

Preparation of vaccines which contain peptide sequences as active ingredients is generally well understood in the art, as exemplified by U.S. Pat. Nos. 4,608,251; 4,601,903; 4,599,231; 4,599,230; 4,596,792; and 4,578,770, all incorporated herein by reference. Typically, such vaccines are prepared as injectables either as liquid solutions or suspensions; solid forms suitable for solution in, or suspension in, liquid prior to injection may also be prepared. The preparation may also be emulsified. The active immunogenic ingredient is often mixed with excipients which are pharmaceutically acceptable and compatible with the active ingredient. Suitable excipients are, for example, water, saline, dextrose, glycerol, ethanol, or the like, and combinations thereof. In addition, if desired, the vaccine may contain minor amounts of auxiliary substances such as wetting or emulsifying agents, pH buffering agents, or adjuvants which enhance the effectiveness of the vaccines; cf. the detailed discussion of adjuvants below.

The vaccines are conventionally administered parenterally, by injection, for example, either subcutaneously, intracutaneously, subdermally or intramuscularly. Additional formulations which are suitable for other modes of administration include suppositories and, in some cases, oral, buccal, sublinqual, intraperitoneal, intravaginal, anal, epidural, spinal, and intracranial formulations. For suppositories, traditional binders and carriers may include, for example, polyalkalene glycols or triglycerides; such suppositories may be formed from mixtures containing the active ingredient in the range of 0.5% to 10%, preferably 1-2%. Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and contain 10-95% of active ingredient, preferably 25-70%. For oral formulations, cholera toxin is an interesting formulation partner (and also a possible conjugation partner).

The polypeptides may be formulated into the vaccine as neutral or salt forms. Pharmaceutically acceptable salts include acid addition salts (formed with the free amino groups of the peptide) and which are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The vaccines are administered in a manner compatible with the dosage formulation, and in such amount as will be therapeutically effective and immunogenic. The quantity to be administered depends on the subject to be treated, including, e.g., the capacity of the individual's immune system to mount an immune response, and the degree of protection desired. Suitable dosage ranges are of the order of several hundred micrograms active ingredient per vaccination with a preferred range from about 0.1 μg to 2,000 μg (even though higher amounts in the 1-10 mg range are contemplated), such as in the range from about 0.5 μg to 1,000 μg, preferably in the range from 1 μg to 500 μg and especially in the range from about 10 μg to 100 μg. Suitable regimens for initial administration and booster shots are also variable but are typified by an initial administration followed by subsequent inoculations or other administrations.

The manner of application may be varied widely. Any of the conventional methods for administration of a vaccine are applicable. These include oral application on a solid physiologically acceptable base or in a physiologically acceptable dispersion, parenterally, by injection or the like. The dosage of the vaccine will depend on the route of administration and will vary according to the age of the person to be vaccinated and the formulation of the antigen.

Some of the variants in the vaccine are sufficiently immunogenic in a vaccine, but for some of the others the immune response will be enhanced if the vaccine further comprises an adjuvant substance.

Various methods of achieving adjuvant effect for the vaccine are known. General principles and methods are detailed in “The Theory and Practical Application of Adjuvants”, 1995, Duncan E. S. Stewart-Tull (ed.), John Wiley & Sons Ltd, ISBN 0-471-95170-6, and also in “Vaccines: New Generation Immunological Adjuvants”, 1995, Gregoriadis G et al. (eds.), Plenum Press, New York, ISBN 0-306-45283-9, both of which are hereby incorporated by reference herein.

It is especially preferred to use an adjuvant which can be demonstrated to facilitate breaking of the autotolerance to autoantigens; in fact, this is essential in cases where unmodified TNF-α is used as the active ingredient in the autovaccine (as discussed above, this is not a preferred embodiment). Non-limiting examples of suitable adjuvants are selected from the group consisting of an immune targeting adjuvant; an immune modulating adjuvant such as a toxin, a cytokine, and a mycobacterial derivative; an oil formulation; a polymer; a micelle forming adjuvant; a saponin; an immunostimulating complex matrix (ISCOM matrix); a particle; DDA; aluminium adjuvants; DNA adjuvants; γ-inulin; and an encapsulating adjuvant. In general it should be noted that the disclosures above which relate to compounds and agents useful as first, second and third moieties in the variants also refer mutatis mutandis to their use in the adjuvant of a vaccine used in the invention.

The application of adjuvants include use of agents such as aluminium hydroxide or phosphate (alum), commonly used as 0.05 to 0.1 percent solution in buffered saline, admixture with synthetic polymers of sugars (e.g. Carbopol®) used as 0.25 percent solution, aggregation of the protein in the vaccine by heat treatment with temperatures ranging between 70° to 101° C. for 30 second to 2 minute periods respectively and also aggregation by means of cross-linking agents are possible. Aggregation by reactivation with pepsin treated antibodies (Fab fragments) to albumin, mixture with bacterial cells such as C. parvum or endotoxins or lipopolysaccharide components of gram-negative bacteria, emulsion in physiologically acceptable oil vehicles such as mannide mono-oleate (Aracel A) or emulsion with 20 percent solution of a perfluorocarbon (Fluosol-DA) used as a block substitute may also be employed. Admixture with oils such as squalene and IFA is also preferred.

According to the invention DDA (dimethyldioctadecylammonium bromide) is an interesting candidate for an adjuvant as is DNA and γ-inulin, but also Freund's complete and incomplete adjuvants as well as quillaja saponins and derivatives such as QuilA and QS21 are interesting as is RIBI. Further possibilities are monophosphoryl lipid A (MPL), the above mentioned C3 and C3d, and muramyl dipeptide (MDP). Also MF59 (marketed by Chiron Corporation) and other MF adjuvants are interesting candidates.

Liposome formulations are also known to confer adjuvant effects, and therefore liposome adjuvants are preferred according to the invention.

Also immunostimulating complex matrix type (ISCOM® matrix) adjuvants are preferred choices according to the invention, especially since it has been shown that this type of adjuvants are capable of up-regulating MHC Class II expression by APCs. An ISCOM® matrix consists of (optionally fractionated) saponins (triterpenoids) from Quillaja saponaria, cholesterol, and phospholipid. When admixed with the immunogenic protein, the resulting particulate formulation is what is known as an ISCOM particle where the saponin constitutes 60-70% w/w, the cholesterol and phospholipid 10-15% w/w, and the protein 10-15% w/w. Details relating to composition and use of immunostimulating complexes can e.g. be found in the above-mentioned text-books dealing with adjuvants, but also Morein B et al., 1995, Clin. Immunother. 3: 461-475 as well as Barr I G and Mitchell G F, 1996, Immunol. and Cell Biol. 74: 8-25 (both incorporated by reference herein) provide useful instructions for the preparation of complete immunostimulating complexes.

Another highly interesting (and thus, preferred) possibility of achieving adjuvant effect is to employ the technique described in Gosselin et al., 1992 (which is hereby incorporated by reference herein). In brief, the presentation of a relevant antigen such as an antigen of the present invention can be enhanced by conjugating the antigen to antibodies (or antigen binding antibody fragments) against the Fcγ receptors on monocytes/macrophages. Especially conjugates between antigen and anti-FcγRI have been demonstrated to enhance immunogenicity for the purposes of vaccination.

Other possibilities involve the use of the targeting and immune modulating substances (i.a. cytokines) mentioned in the claims as moieties for the protein constructs. In this connection, also synthetic inducers of cytokines like poly I:C are possibilities.

Suitable mycobacterial derivatives are selected from the group consisting of muramyl dipeptide, complete Freund's adjuvant, RIBI, and a diester of trehalose such as TDM and TDE.

Suitable immune targeting adjuvants are selected from the group consisting of CD40 ligand and CD40 antibodies or specifically binding fragments thereof (cf. the discussion above), mannose, a Fab fragment, and CTLA-4.

Suitable polymer adjuvants are selected from the group consisting of a carbohydrate such as dextran, PEG, starch, mannan, and mannose; plastic polymers; and latex such as latex beads.

Yet another interesting way of modulating an immune response is to include the immunogen (optionally together with adjuvants and pharmaceutically acceptable carriers and vehicles) in a “virtual lymph node” (VLN) (a proprietary medical device developed by ImmunoTherapy, Inc., 360 Lexington Avenue, New York, N.Y. 10017-6501). The VLN (a thin tubular device) mimics the structure and function of a lymph node. Insertion of a VLN under the skin creates a site of sterile inflammation with an upsurge of cytokines and chemokines. T- and B-cells as well as APCs rapidly respond to the danger signals, home to the inflamed site and accumulate inside the porous matrix of the VLN. It has been shown that the necessary antigen dose required to mount an immune response to an antigen is reduced when using the VLN and that immune protection conferred by vaccination using a VLN surpassed conventional immunization using Ribi as an adjuvant. The technology is i.a. described briefly in Gelber C et al., 1998, “Elicitation of Robust Cellular and Humoral Immune Responses to Small Amounts of Immunogens Using a Novel Medical Device Designated the Virtual Lymph Node”, in: “From the Laboratory to the Clinic, Book of Abstracts, Oct. 12^{th}-15^{th} 1998, Seascape Resort, Aptos, Calif.”. It is worth noting that this particular mode of the present invention possibly could be used without the need for a modified TNF-α, but merely utilising a strongly adjuvated TNF-α.

It is expected that the vaccine should be administered at least once a year, such as at least 1, 2, 3, 4, 5, 6, and 12 times a year. More specifically, 1-12 times per year is expected, such as 1, 2, 3, 4, 5, 6, 7, 8, 9, 10, 11, or 12 times a year to an individual in need thereof. It has previously been shown that the memory immunity induced by the use of the preferred autovaccines according to the invention is not permanent, and therefor the immune system needs to be periodically challenged with the variants. The immunization scheme can be discontinued at any time resulting in a decline in antibody titers after a relatively short while.

Due to genetic variation, different individuals may react with immune responses of varying strength to the same polypeptide. Therefore, the vaccine according to the invention may comprise several different polypeptides in order to increase the immune response, cf. also the discussion above concerning the choice of foreign T-cell epitope introductions. The vaccine may comprise two or more polypeptides, where all of the polypeptides are as defined above.

The vaccine may consequently comprise 3-20 different variants, such as 3-10 variants. However, normally the number of variants will be sought kept to a minimum such as 1 or 2 variants.

Nucleic Acid Vaccination

As a very important alternative to classic administration of a peptide-based vaccine, the technology of nucleic acid vaccination (also known as “nucleic acid immunisation”, “genetic immunisation”, and “gene immunisation”) offers a number of attractive features.

First, in contrast to the traditional vaccine approach, nucleic acid vaccination does not require resource consuming large-scale production of the immunogenic agent (e.g. in the form of industrial scale fermentation of microorganisms producing proteins). Furthermore, there is no need to device purification and refolding schemes for the immunogen. And finally, since nucleic acid vaccination relies on the biochemical apparatus of the vaccinated individual in order to produce the expression product of the nucleic acid introduced, the optimum posttranslational processing of the expression product is expected to occur; this is especially important in the case of autovaccination, since, as mentioned above, a significant fraction of the original B-cell epitopes of the polymer should be preserved in the modified molecule, and since B-cell epitopes in principle can be constituted by parts of any (bio)molecule (e.g. carbohydrate, lipid, protein etc.). Therefore, native glycosylation and lipidation patterns of the immunogen may very well be of importance for the overall immunogenicity and this is expected to be ensured by having the host producing the immunogen.

It should be noted that the enhanced expression levels reported for monomeric “multimer mimics” of IL5 in DK PA 2001 01702 render especially that type of TNFα multimer mimics interesting because this high expression level is very important for efficacy of DNA vaccination, as the in vivo expression level is one of the determining factors in the immunogenic efficacy of a DNA vaccine

Hence, a preferred embodiment of the invention comprises effecting presentation of TNF-α-variant of the invention to the immune system by introducing nucleic acid(s) encoding the variant into the animal's cells and thereby obtaining in vivo expression by the cells of the nucleic acid(s) introduced.

In this embodiment, the introduced nucleic acid is preferably DNA which can be in the form of naked DNA, DNA formulated with charged or uncharged lipids, DNA formulated in liposomes, DNA included in a viral vector, DNA formulated with a transfection-facilitating protein or polypeptide, DNA formulated with a targeting protein or polypeptide, DNA formulated with Calcium precipitating agents, DNA coupled to an inert carrier molecule, DNA encapsulated in a polymer, e.g. in PLGA (cf. the microencapsulation technology described in WO 98/31398) or in chitin or chitosan, and DNA formulated with an adjuvant. In this context it is noted that practically all considerations pertaining to the use of adjuvants in traditional vaccine formulation apply for the formulation of DNA vaccines. Hence, all disclosures herein which relate to use of adjuvants in the context of polypeptide based vaccines apply mutatis mutandis to their use in nucleic acid vaccination technology.

As for routes of administration and administration schemes of polypeptide based vaccines which have been detailed above, these are also applicable for the nucleic acid vaccines used in the invention and all discussions above pertaining to routes of administration and administration schemes for polypeptides apply mutatis mutandis to nucleic acids. To this should be added that nucleic acid vaccines can suitably be administered intraveneously and intraarterially. Furthermore, it is well-known in the art that nucleic acid vaccines can be administered by use of a so-called gene gun, and hence also this and equivalent modes of administration are regarded as part of the present invention. Finally, also the use of a VLN in the administration of nucleic acids has been reported to yield good results, and therefore this particular mode of administration is particularly preferred.

Furthermore, the nucleic acid(s) used as an immunization agent can contain regions encoding the moieties specified above, e.g. in the form of the immunomodulating substances described above such as the cytokines discussed as useful adjuvants. A preferred version of this embodiment encompasses having the coding region for the TNF-α variant and the coding region for the immunomodulator in different reading frames or at least under the control of different promoters. Thereby it is avoided that the variant or epitope is produced as a fusion partner to the immunomodulator. Alternatively, two distinct nucleotide fragments can be used, but this is less preferred because of the advantage of ensured co-expression when having both coding regions included in the same molecule.

Under normal circumstances, the nucleic acid is introduced in the form of a vector wherein expression is under control of a viral promoter. For more detailed discussions of vectors and DNA fragments according to the invention, cf. the discussion below. Also, detailed disclosures relating to the formulation and use of nucleic acid vaccines are available, cf. Donnelly J J et al, 1997, Annu. Rev. Immunol. 15: 617-648 and Donnelly J J et al., 1997, Life Sciences 60: 163-172. Both of these references are incorporated by reference herein.

Live Vaccines

A third alternative for effecting presentation of the TNF-α variants to the immune system is the use of live vaccine technology. In live vaccination, presentation to the immune system is effected by administering, to the animal, a non-pathogenic microorganism that has been transformed with a nucleic acid fragment encoding a TNF-α variant as described herein or with a vector incorporating such a nucleic acid fragment. The non-pathogenic microorganism can be any suitable attenuated bacterial strain (attenuated by means of passaging or by means of removal of pathogenic expression products by recombinant DNA technology), e.g. Mycobacterium bovis BCG., non-pathogenic Streptococcus spp., E. coli, Salmonella spp., Vibrio cholerae, Shigella, etc. Reviews dealing with preparation of state-of-the-art live vaccines can e.g. be found in Saliou P, 1995, Rev. Prat. 45: 1492-1496 and Walker P D, 1992, Vaccine 10: 977-990, both incorporated by reference herein. For details about the nucleic acid fragments and vectors used in such live vaccines, cf. the discussion below.

As an alternative to bacterial live vaccines, the nucleic acid fragment discussed below can be incorporated in a non-virulent viral vaccine vector such as a vaccinia strain or any other suitable pox virus.

Normally, the non-pathogenic microorganism or virus is administered only once to the animal, but in certain cases it may be necessary to administer the microorganism more than once in a lifetime in order to maintain protective immunity. It is even contemplated that immunization schemes as those detailed above for polypeptide vaccination will be useful when using live or virus vaccines.

Alternatively, live or virus vaccination is combined with previous or subsequent polypeptide and/or nucleic acid vaccination. For instance, it is possible to effect primary immunization with a live or virus vaccine followed by subsequent booster immunizations using the polypeptide or nucleic acid approach.

The microorganism or virus can be transformed with nucleic acid(s) containing regions encoding the moieties mentioned above, e.g. in the form of the immunomodulating substances described above such as the cytokines discussed as useful adjuvants. A preferred version of this embodiment encompasses having the coding region for the variant and the coding region for the immunomodulator in different reading frames or at least under the control of different promoters. Thereby it is avoided that the variant or epitopes are produced as fusion partners to the immunomodulator. Alternatively, two distinct nucleotide fragments can be used as transforming agents. Of course, having the adjuvating moieties in the same reading frame can provide, as an expression product, a TNF-α variant useful in the invention, and such an embodiment is especially preferred according to the present invention.

Combination Treatment

One especially preferred mode of carrying out the invention involves the use of nucleic acid vaccination as the first (primary) immunization, followed by secondary (booster) immunizations with a polypeptide based vaccine as described above.

Compositions Useful in the Invention

The invention also utilises immunogenic compositions comprising an immunogenically effective amount of a TNF-α variant described above, said composition further comprising a pharmaceutically and immunologically acceptable diluent and/or vehicle and/or carrier and/or excipient and optionally an adjuvant—in other words formulations of variants, essentially as described hereinabove. The choice of adjuvants, carriers, and vehicles is accordingly in line with what has been discussed above when referring to formulation of the variants for peptide vaccination.

The variants are prepared according to methods well-known in the art. Longer polypeptides are normally prepared by means of recombinant gene technology including introduction of a nucleic acid sequence encoding the variant into a suitable vector, transformation of a suitable host cell with the vector, expression of the nucleic acid sequence, recovery of the expression product from the host cells or their culture supernatant, and subsequent purification and optional further modification, e.g. refolding or derivatization.

Shorter peptides are preferably prepared by means of the well-known techniques of solid- or liquid-phase peptide synthesis. However, recent advances in this technology has rendered possible the production of full-length polypeptides and proteins by these means, and therefore it is also within the scope of the present invention to prepare the long constructs by synthetic means.

Nucleic Acid Fragments and Vectors Useful in the Invention

It will be appreciated from the above disclosure that modified polypeptides can be prepared by means of recombinant gene technology but also by means of chemical synthesis or semisynthesis; the latter two options are especially relevant when the modification of TNF-α consists of or comprises coupling to protein carriers (such as KLH, diphtheria toxoid, tetanus toxoid, and BSA) and non-proteinaceous molecules such as carbohydrate polymers and of course also when the modification comprises addition of side chains or side groups to an polymer-derived peptide chain.

For the purpose of recombinant gene technology, and of course also for the purpose of nucleic acid immunization, nucleic acid fragments encoding the variants are important chemical products. Hence, an important part of the invention pertains to the use of a nucleic acid fragment which encodes a TNF-α variant as described herein. The nucleic acid fragments of the invention are either DNA or RNA fragments.

The nucleic acid fragments useful in the invention will normally be inserted in suitable vectors to form cloning or expression vectors carrying the nucleic acid fragments; such novel vectors are also useful in the novel uses according to the invention. Details concerning the construction of these vectors will be discussed in context of transformed cells and microorganisms below. The vectors can, depending on purpose and type of application, be in the form of plasmids, phages, cosmids, mini-chromosomes, or virus, but also naked DNA, which is only expressed transiently in certain cells, is an important vector (and may be useful in DNA vaccination). Preferred cloning and expression vectors useful in the invention are capable of autonomous replication, thereby enabling high copy-numbers for the purposes of high-level expression or high-level replication for subsequent cloning.

The general outline of a vector useful in the invention comprises the following features in the 5′→3′ direction and in operable linkage: a promoter for driving expression of the nucleic acid fragment, optionally a nucleic acid sequence encoding a leader peptide enabling secretion (to the extracellular phase or, where applicable, into the periplasma) of or integration into the membrane of the polypeptide fragment, the nucleic acid fragment useful in the invention, and optionally a nucleic acid sequence encoding a terminator. When operating with expression vectors in producer strains or cell-lines it is for the purposes of genetic stability of the transformed cell preferred that the vector when introduced into a host cell is integrated in the host cell genome. In contrast, when working with vectors to be used for effecting in vivo expression in an animal (i.e. when using the vector in DNA vaccination) it is for security reasons preferred that the vector is not capable of being integrated in the host cell genome; typically, naked DNA or non-integrating viral vectors are used, the choices of which are well-known to the person skilled in the art.

The vectors useful in the invention are used to transform host cells to produce the TNF-α variants. Such transformed cells, which are also useful tools for practicing the invention, can be cultured cells or cell lines used for propagation of the nucleic acid fragments and vectors useful in the invention, or used for recombinant production of the modified TFN-α polypeptides. Alternatively, the transformed cells can be suitable live vaccine strains wherein the nucleic acid fragment (one single or multiple copies) have been inserted so as to effect secretion or integration into the bacterial membrane or cell-wall of the modified TNF-α.

Preferred transformed cells useful in the invention are microorganisms such as bacteria (such as the species Escherichia (e.g. E. coli), Bacillus [e.g. Bacillus subtilis], Salmonella, or Mycobacterium [preferably non-pathogenic, e.g. M. bovis BCG]), yeasts (such as Saccharomyces cerevisiae), and protozoans. Alternatively, the transformed cells are derived from a multicellular organism such as a fungus, an insect cell, a plant cell, or a mammalian cell. Most preferred are cells derived from a human being, cf. the discussion of cell lines and vectors below. Recent results have shown great promise in the use of a commercially available Drosophila melanogaster cell line (the Schneider 2 (S₂) cell line and vector system available from Invitrogen) for the recombinant production of TNF-α variants used in the invention, and therefore this expression system is particularly preferred, and therefore this type of system is also part of a preferred embodiment of the invention in general.

For the purposes of cloning and/or optimized expression it is preferred that the transformed cell is capable of replicating the nucleic acid fragment useful in the invention. Cells expressing the nucleic fragment are parts of preferred useful embodiments of the invention; they can be used for small-scale or large-scale preparation of the variant or, in the case of non-pathogenic bacteria, as vaccine constituents in a live vaccine.

When producing the TNF-α variants by means of transformed cells, it is convenient, although far from essential, that the expression product is either exported out into the culture medium or carried on the surface of the transformed cell, since both of these options facilitate subsequent purification of the expression product.

When an effective producer cell has been identified it is preferred, on the basis thereof, to establish a stable cell line which carries the vector useful in the invention and which expresses the nucleic acid fragment encoding the variant TNF-α. Preferably, this stable cell line secretes or carries the variant, thereby facilitating purification thereof.

In general, plasmid vectors containing replicon and control sequences that are derived from species compatible with the host cell are used in connection with the hosts. The vector ordinarily carries a replication site, as well as marking sequences which are capable of providing phenotypic selection in transformed cells. For example, E. coli is typically transformed using pBR322, a plasmid derived from an E. coli species (see, e.g., Bolivar et al., 1977). The pBR322 plasmid contains genes for ampicillin and tetracycline resistance and thus provides easy means for identifying transformed cells. The pBR plasmid, or other microbial plasmid or phage must also contain, or be modified to contain, promoters that can be used by the prokaryotic microorganism for expression.

Those promoters most commonly used in prokaryotic recombinant DNA construction include the B-lactamase (penicillinase) and lactose promoter systems (Chang et al., 1978; Itakura et al., 1977; Goeddel et al., 1979) and a tryptophan (trp) promoter system (Goeddel et al., 1979; EP-A-0 036 776), and also the T7 promoter system has proven very effective. While these are the most commonly used, other microbial promoters have been discovered and utilized, and details concerning their nucleotide sequences have been published, enabling a skilled worker to ligate them functionally with plasmid vectors (Siebwenlist et al., 1980). Certain genes from prokaryotes may be expressed efficiently in E. coli from their own promoter sequences, precluding the need for addition of another promoter by artificial means.

In addition to prokaryotes, eukaryotic microbes, such as yeast cultures may also be used, and here the promoter should be capable of driving expression. Saccharomyces cerevisiase, or common baker's yeast is the most commonly used among eukaryotic microorganisms, although a number of other strains are commonly available. For expression in Saccharomyces, the plasmid YRp7, for example, is commonly used (Stinchcomb et al., 1979; Kingsman et al., 1979; Tschemper et al., 1980). This plasmid already contains the trpl gene which provides a selection marker for a mutant strain of yeast lacking the ability to grow in tryptophan for example ATCC No. 44076 or PEP4-1 (Jones, 1977). The presence of the trpl lesion as a characteristic of the yeast host cell genome then provides an effective environment for detecting transformation by growth in the absence of tryptophan.

Suitable promoting sequences in yeast vectors include the promoters for 3-phosphoglycerate kinase (Hitzman et al., 1980) or other glycolytic enzymes (Hess et al., 1968; Holland et al., 1978), such as enolase, glyceraldehyde-3-phosphate dehydrogenase, hexokinase, pyruvate decarboxylase, phosphofructo-kinase, glucose-6-phosphate isomerase, 3-phosphoglycerate mutase, pyruvate kinase, triosephosphate isomerase, phosphoglucose isomerase, and glucokinase. In constructing suitable expression plasmids, the termination sequences associated with these genes are also ligated into the expression vector 3′ of the sequence desired to be expressed to provide polyadenylation of the mRNA and termination.

Other promoters, which have the additional advantage of transcription controlled by growth conditions are the promoter region for alcohol dehydrogenase 2, isocytochrome C, acid phosphatase, degradative enzymes associated with nitrogen metabolism, and the aforementioned glyceraldehyde-3-phosphate dehydrogenase, and enzymes responsible for maltose and galactose utilization. Any plasmid vector containing a yeast-compatible promoter, origin of replication and termination sequences is suitable.

In addition to microorganisms, cultures of cells derived from multicellular organisms may also be used as hosts. In principle, any such cell culture is workable, whether from vertebrate or invertebrate culture. However, interest has been greatest in vertebrate cells, and propagation of vertebrate in culture (tissue culture) has become a routine procedure in recent years (Tissue Culture, 1973). Examples of such useful host cell lines are VERO and HeLa cells, Chinese hamster ovary (CHO) cell lines, and W138, BHK, COS-7 293, Spodoptera frugiperda (SF) cells (commercially available as complete expression systems from i.a. Protein Sciences, 1000 Research Parkway, Meriden, Conn. 06450, U.S.A. and from Invitrogen), and MDCK cell lines. In the present invention, an especially preferred cell line the insect cell line S₂, available from Invitrogen, PO Box 2312, 9704 CH Groningen, The Netherlands.

Expression vectors for such cells ordinarily include (if necessary) an origin of replication, a promoter located in front of the gene to be expressed, along with any necessary ribosome binding sites, RNA splice sites, polyadenylation site, and transcriptional terminator sequences.

For use in mammalian cells, the control functions on the expression vectors are often provided by viral material. For example, commonly used promoters are derived from polyoma, Adenovirus 2, and most frequently Simian Virus 40 (SV40) or cytomegalovirus (CMV). The early and late promoters of SV40 virus are particularly useful because both are obtained easily from the virus as a fragment which also contains the SV40 viral origin of replication (Fiers et al., 1978). Smaller or larger SV40 fragments may also be used, provided there is included the approximately 250 bp sequence extending from the HindIII site toward the BglI site located in the viral origin of replication. Further, it is also possible, and often desirable, to utilize promoter or control sequences normally associated with the desired gene sequence, provided such control sequences are compatible with the host cell systems.

An origin of replication may be provided either by construction of the vector to include an exogenous origin, such as may be derived from SV40 or other viral (e.g., Polyoma, Adeno, VSV, BPV) or may be provided by the host cell chromosomal replication mechanism. If the vector is integrated into the host cell chromosome, the latter is often sufficient.

The present invention will now be illustrated by means of the following, non-limiting examples.

### PREAMBLE TO EXAMPLES

Animal Model

Male Sprauge-Dawley rats (200-600 g) were used in all the experiments. The rats were permitted to acclimate 1 week before use in the experiments. The rats were marked with a speed marker on their tails and normally kept in groups of 2-3 per cage pre- and postoperatively. They were housed in clear plastic cages with solid floors covered with sawdust in a normal day/night cycle. Water and food were supplied ad libitum.

Anaesthesia and Analgesia

For the partial sciatic nerve injury and the chronic constriction injury model, surgery was performed under general anaesthesia using 4-5% of either Isoflurane or Sevoflurane mixed with 70% nitrogen and 30% oxygen. For the induction, rats were placed in a box continuously perfusated with the gas mixture. Afterwards, anaesthesia was maintained by an open mask system. The rats were awake within 5 minutes after the anaesthesia was discontinued. No local analgesia was used. The rats were kept on a thermo pad during surgery to preserve body temperature, no thermometer was used since the surgical procedure only lasted for 15-20 min.

For the insertion of guide cannulas, brain infusion cannula and osmotic pumps, a mixture of Hypnorm-Dormicum water was used. Prepared as 1 part hypnorm mixed with 1 part sterile water, 1 part dormicum mixed with 1 part sterile water, the two solutions are then mixed together. It is important to keep the sequence of preparation otherwise the mixture will precipitate. The rats receive a dosage of 2 ml/kg+0.2 ml, which provides a deep anaesthesia for 2-3 hours and an analgesic effect of app. 50 min. The surgical procedure lasted for 40-50 min. A thermometer was used in combination with a thermo pad to prevent hypothermia. To prevent the rats from choking and to ease respiration, the tongue was gently pulled out with a forceps both during surgery and in the recovery period. Post surgery, the rats received 5-6 ml saline SC and Temgesic 0.15 ml/kg SC. The rats received also Temgesic for 3 days in their drinking water (16 ml/l).

Before any surgical procedure began, the lack of interdigital, palpebral and corneal reflexes was checked.

Surgical Procedures

Partial Sciatic Nerve Ligation.

The method of partial sciatic nerve injury is developed by Seltzer. Before surgery, the rats were shaved and washed with iodine solution (Iobac vet.) on the surgical area. The left sciatic nerve was exposed at high-thigh level by making a 2-3 cm incision in the skin. The skin was then freed from the connective tissue surrounding the incision in order to assure free movement of the leg after skin stapling. Using blunt dissection through the biceps femoris, the nerve was exposed and carefully freed from surrounding connective tissue at a site near the trochanter just below the point at which the posterior biceps semitendinosus nerve branches off the common sciatic nerve. Using a small pincette slid under the nerve, the nerve was gently lifted up and fixed. An 8/0 Ethilon suture was inserted into the nerve and tightly ligated so that the dorsal 1/3-1/2 of the nerve thickness was trapped in the ligature. The muscle was closed with 2-3 muscle sutures (4/0 vicryl), and the skin closed with 3-4 skin staples.

Chronic Constriction Injury.

The method of chronic constriction injury (CCI) is developed by Bennett and Xie{1}. The surgical procedure as described above was basically used. The incision was made mid-thigh and the nerve was exposed again by blunt dissection through biceps femoris. Proximal to the common sciatic nerve's trifurcation, the nerve was freed from surrounding connective tissue, lifted and fixed with a small pincette as described above. 4 ligatures (4.0 cat gut chromic suture, Ethicon) were tied loosely around the nerve with a single surgical knot and about 1 mm spacing. When the ligatures were in place, a microscope using 40× magnification was used during adjustment of the ligatures.

It is of crucial importance that the ligatures are tied in a manner so that they barely constrict the nerve. If the ligatures are made to tight, there will be a high risk of autotomy. On the other hand, if the ligatures are made to loose, the rats will not develop the neuropathic pain state. A good ligature will be moveable only at the top, if very gently pulled back and forth with a microsurgical forceps, while the bottom part will stay in place.

Once the ligatures were adjusted, the wound was closed as described above.

Insertion of Osmotic Minipumps and Brain Infusion Cannula

This procedure is described very thoroughly in the manufactures' instructions and specifications sheet for the Brain Infusion Kit (Alzet) which was used in these experiments. Stereotaxic coordinates for intracerebroventricular microinfusion into the right lateral cerebral ventricle were given with bregma used as the zero point as: anterior-posterior (A-P) −0.92 mm, lateral 1.6 mm, vertical 3.5 mm for trephining the skull prior to cannulation. For an overview of microinfusion with osmotic minipumps, see White et al., 1995.

The mini-osmotic pumps (model 2002, Alzet) with catheter tubes were filled according to the manufacturer's guidelines, then submerged in sterile 0.9% saline and kept on 37° C. water bath 1 day prior to surgery. This assured that the pumps were activated and that the catheter tubes were free of air at the insertion time. The pumps delivered 0.5 μl/hr for 14 days.

Before surgery, the rats scalps were shaved and washed with iodine solution (Iobac vet.). The rats were secured on a stereotaxic platform. A midline sagittal incision was made starting slightly behind the eyes and approximately 2.5 cm long exposing the scull. 2 small clamps were attached to each side of the incision to pull away the skin from the scull. 1 drop of Xylocain (2%) was dropped on the periosteal connective tissue before scraping of the tissue from the scull. The scull area exposed was scraped to stop bleeding and patted dry.

From the end of the incision to the upper back of the rat, a subcutaneous pocket was created using blunt dissection with a hemostat. The pocket was widened at the bottom to accommodate the pump, making just enough room for the pump to move, but not enough to let it slip down onto the rats flank. The osmotic pump was then inserted into the pocket and the catheter attached to the cannula after adjusting the length of the catheter, assuring free motion of the rats' neck and head.

With bregma as the reference point, the stereotaxic coordinates were used to determine the location for cannula placement. The location was marked and a small hole (1 mm diameter) was drilled in the scull. This hole was later receiving the guide cannula. 3 mm posterior to the first hole and 4 mm to the left and right of it, two more very small holes (0.5 mm diameter) were drilled. Stainless steel screws were secured into these holes with one or two turns. The screws act as an anchor for the dental cement to the scull.

Using the stereotaxic instrument, the cannula was then inserted through the skull to the correct depth, over a period of 2-3 min to minimize damage of the brain. Once in place, the scull was completely dried and dental cement was applied, covering the anchor screws and the entire implantation site up to the placement tab of the cannula. Care was taken not to make sharp edges on the cement surface. The dental cement was allowed to dry for 5 min, the cannula placement tab was released from the stereotaxic instrument and cut off. The wound was then sutured with interrupted horizontal mattress suture (4-0 Vicryl). Xylocain ointment (5%) was applied on the wound surface.

Insertion of Guide Cannulas and Injection Technique

For the insertion of guide cannulas, the same method and coordinates as described above were used. After insertion of the cannula and closure of the wound, a top piece was inserted into the cannula to secure it from clotting. After a recovery period of 5-10 days, the animals were ready to use.

When giving an icv injection, the top piece was removed and a very thin cannula, connected to a 25 μl Hamilton syringe by a 20 cm catheter, was inserted into the guide cannula. The rat was then placed back into its cage and 5 μl of injection fluid was given over 2 min. The cannula was then removed and the top piece reinserted. This is a very god method for icv injection, because it only requires a minimum of restraint causing less stress for the rat.

Vaccination

Vaccination Protocol

For the vaccination with an immunogenic TNF-α (variant, rats aged 6 weeks at the primary immunization were used. Each vaccination dosage contained 50 μg of modified murine monomeric TNF-α protein (TNF-α 106, SEQ ID NO: 5) and 10 μg of Quil-A adjuvant dissolved in isotonic saline for a total volume of 100 μl. The control group received 10 μg of Quil-A adjuvant dissolved in isotonic saline (Sigma) for a total volume of 100 μl. The injections were given subcutaneously at the base of the tail, which is a very spongiform tissue, assuring a slow systemical release. Following the first immunization, the rats were boosted with the same dosage every second week until the end of the experiments. Experiments began after four immunizations, when sufficiently high titers were achieved.

Blood Sampling

Blood samples obtained from the orbital plexus were obtained after the first four immunizations. The rats were anaesthetized in a box perfusated with 5-6% of Sevoflurane mixed with 70% nitrogen and 30% oxygen. The anaesthetized rat was removed from the box and stasis was obtained by gently grasping the scruff. The conjuctiva and underlying tissues of the canthus of the eye were perforated with a standard hematocrit tube. The tube was pushed through the tissues while it was gently rotated until the plexus was reached. 0.5 to 1 ml of blood was collected. The stasis was released before the tube was removed. The rats eye was wiped and closed before the rat was put back into its cage. The blood sampling can be done within 1 minute, leaving the rat anaesthetized during the whole procedure. The next day, the rat's eye was checked for irregularities. If swelling occurred, the rat was euthanasized.

Blood samples were left to coagulate for 1 hour before centrifugating at 3600 rpm for 10 minutes. Serum was extracted and refrigerated until used.

Immunocytochemistry for NF-κB

Preparation and Storage of Tissue

The rat was anaesthetized with Avertin (20 mg/ml) given a dosage of 200 mg/kg. Before any surgical procedure began, the lack of interdigital, palpebral and corneal reflexes was checked. The thorax of the rat was opened and through an incision in the bottom of the left ventricle, a catheter was inserted into the aorta. The perfusation was started at a rate of 20 ml/min, first for 2 minutes with KPBS pH 7.4 (containing 3 ml of Heparin per liter in order to prevent blood from clotting) to drain the rat for blood, then for 10 minutes with 0.4% paraformaldehyde to fixate the rat.

The brains were post fixed in the same fixative. 1 day before sectioning, the brains were cryoprotected in 30% sucrose solution in KPBS. The brains were frozen and cut in sections at 40 mm. The sections were stored in the plastic tubes, containing KPBS, in which the reaction was taken place (free floating reaction).

Procedure

Immunocytochemistry was conducted in accordance with the ABC-P METHOD PMC (Avidin Biotin Bridge Method with Peroxidase Substrate).
1. Rinse of sections: 3×10 minutes in 50 mM KPBS (pH 7.2 -7.4).
2. Blocking of endogenous perioxidase: Incubation of sections for 10 minutes in 1% H₂O₂ in KPBS.
3. Blocking step: Incubation of sections for 20 minutes in 5.0% Normal serum (porcine) (NS) in KPBS+0.3% Triton-X-100 (TX)+1.0% Bovine Serum Albumine (BSA, SIGMA).
4. Incubation of sections with polyclonal antibody NFκB p65 (Santa Cruz) diluted (1/250) in KPBS+0.3% TX+1.0% BSA at 4° C. in covered plastic tubes for 48 hours.
5. Rinse of sections: 3×10 minutes in KPBS+0.1% TX+0.25% BSA.
6. Incubation with the 2⁰ antibody goat anti-rabit IgG E353 (Dakopatts) for 60 min diluted (1/500) in KPBS+0.3% TX+1.0% BSA.
7. Rinse of sections: 2×10 minutes in KPBS+0.1% TX+0.25% BSA.
8. Rinse: 1×10 minutes in KPBS.
9. Incubation in Avidin:Biotin:Peroxidase Complex, (Vector kit, ABC Elitekit Vector Pre 6100): 1 drop A+1 drop B in 25 ml KPBS. Mixed 30 minutes before use, for 60 minutes.
10. Rinse: 2×10 minutes in KPBS.
11. Rinse: 10 minutes in TRIS Buffer.
12. Development of sections in Chromagen Solution for 10 minutes (0.04% diamiobenzidine tetrahydrchloride(DAB)+0.003% H₂O₂ in Tris buffer).
13. The sections were rinsed 2×10 minutes in distilled water and finally mounted onto glass slides.
   Measurement of Hyperalgesia.

Plantar test (Ugo Basile) was used in this study to asses the nociceptive response.

The rats were placed in the clear plastic chamber (18 cm×29 cm×12.5 cm) allowed to acclimate to the new environment for about 5 min. Before testing. During this time period the rat was showing exploratory behaviour e.g. standing on its hind leg. When the rat had been acclimated, it stood quietly with occasional bouts of grooming. The radiant heat score beneath the class floor was pointed at the planter hind paw with help from the white sight marks engraved on the top of the I.R vessel.

The infrared (IR) source was then started and the withdrawal latency recorded.

When the rat felt pain and withdrew its paw, the sudden drop in radiation led to a automatic stop of the IR source and the time counter and the withdrawal latency could be determined to the nearest 0.1 sec.

The withdrawal latency was every time tested 5 times with intervals of 5 min. The average of the 5 measurements were then used as the withdrawal latency of the specific rat.

Rats, which were undergoing PSNL, were only tested on the operated left leg. 3 consecutive days before the operation, the withdrawal latency of the left leg was measured using the average of the 3 days as the basic withdrawal latency. The rat was then tested postoperatively for more days.

The difference score, expressed as percent reduction from the basic level to the postoperative level measurement, was used as an index for hyperalgesia:

$$hyperalgesia ( difference score ) = \frac{basic level - pom}{basic level} \times 100 \%$$

(pom: postoperative measurement)

Animals, which were undergoing the CCI operation, were tested one day before the operation with the purpose to make sure that the 2 groups were not significantly different. The rats were tested 4 times on both hind paws using average measurements as the withdrawal latency of the individual rat.

The difference score expressed as percent difference between the withdrawal latency of left and right hind paws was used as an index for hyperalgesia in this model.

$$hyperalgesia ( difference score ) = \frac{moul - pom}{moul} \times 100 \%$$

(moul: measurement of unoperated leg; pom: postoperative measurement)
Statistics

Group averages appear with the standard error of the mean. The significance level of difference between means of various groups at certain postoperative times was examined by Student's t-test. The difference between the same group but at different days was examined by ANOVA.

### Example 1

Verifying Induction of Thermal Hyperalgesia by the Nerve Ligation in the PSNI Model

6 rats were used in the shame group and 10 in the PSNL group. The rats were operated in accordance with the operation procedure but without ligating the nerve in the shame group.

Rats were tested days 2, 4, 6, 8 and 10 postoperatively.

Nociceptive threshold for latency to hind paw withdrawal in the PSNI model and in a sham operated group is shown in FIG. 1: Data are expressed as the difference score in percent between the basic level and the postoperative measurement. Each point represents mean±SEM, and the number of determinations are indicated in parenthesis. Significant difference between the two groups P<0.05 (Student's t-test) is marked with “*”.

There is significant difference between the sham and PSNL operated groups at all days measured. (t-test): day 2 (sham 6.2±3.9: control 25.9±6.5, P<0.05); day 4 (sham −0.5±5.1: control 32.5±3.2, P<0.001); day 6 (sham 1.7±6.0: control 23.3±3.6, P<0.01); day 8 (sham 5.5±4.1: control 32.7±4.5, P<0.01); day 10 (sham −2.0±8.0: control 21.2±4.7, P<0.05).

There is no statistically significant difference (P>0.05) within the same groups (control and sham, respectively) at the measured days (ANOVA).

### Example 2

Assessment of Effect of Peripheral mAB Administration on Thermal Hyperalgesia

In order to clarify whether infliximab is able to reduce thermal hyperalgesia when administrated i.v. peripherally, this experiment was conducted. There is no previously conducted study where the PSNI model has been used. A previous study has shown a beneficial effect in reducing thermal hyperalgesia of peripherally administrated TFN-α antibody in the CCI model, cf. above.

12 rats (200-250 g) received Remicade (infliximab) 5 mg/kg i.v. 2 hours prior to PSNL operation. 12 rats were used in the control group.

Rats were tested at days 2, 4, 6 and 7 postoperatively.

FIG. 2 shows the nociceptive threshold expressed as latency to hind paw withdrawal in the group receiving infliximab (5 mg/kg) and in the group receiving saline. Data are expressed as the difference score in percent between the basic level and the postoperative measurement. Each point represents mean±SEM, and the number of rats is indicated in the parenthesis

No significant differences in difference score were found between the two groups at any of the measured days. There is no statistically significant difference (P>0.05) within the same groups both in the control and infliximab groups at the measured days (ANOVA).

### Example 3

Effect of Thalidomide on Hyperalgesia in the PSNI Model

A previous study has shown that Thalidomide administrated to rats is able to reduce thermal hyperalgesia induced by CCI model. It is therefore investigated if this is also the case with use of the PSNI model.

2 groups of 10 rats (200-250 g) received 50 and 100 mg/kg thalidomide, respectively. Thalidomide was administrated orally in a sesame oil suspension containing 10 mg/ml starting two hours prior to the operation and continued with one administration each of the following days in the experiment. The control group (n=10) only received the sesame oil. Rats were tested at days 2, 4, 6, 8 and 10 postoperatively. The experiment was blinded in a way so the surgeon did not know the composition administered to each individual rat.

FIG. 3 shows nociceptive threshold for latency to hind paw withdrawal in a control group and to thalidomide administrated groups. Data are expressed as the difference score in percent between the basic level and the postoperative measurements. Each point represents mean±S.E.M, and the number of rats in each is indicated in the parenthesis. day 2 (control 24.6±7.1%: 50 mg/kg 33.3±4.1%: 100 mg/kg 22.0±7.2%, P>0.05); day 4 (control 32.5±3.2%: 50 mg/kg 33.9±5.9%: 100 mg/kg 32.4±6.4%, P>0.05); day 6 (control 23.3±3.6%: 50 mg/kg 40.5±3.2%: 100 mg/kg 38.0±4.1 P<0.05); day 8 (control 32.7±4.1%: 50 mg/kg 41.3±3.4%: 100 mg/kg 34.1±5.4, P>0.05); day 10 (control 21.2±4.7%: 50 mg/kg 30.6±5.3%: 100 mg/kg 29.8±4.6%, P>0.05).

There are significant differences between the control group and both of the thalidomide-administrated groups at day 6 with a lower difference score in the control group.

There is no statistically significant difference (P>0.05) in the mean of the same group both in the control and thalidomide groups at the different days measured (ANOVA).

### Example 4

Effect of Post-Surgical ICV Infusion of Anti-TNF-α with Osmotic Pump on Reducing Pain in the PSNI Model

ICV infusion of TNF-α antibodies with osmotic pump has previously proven successful in reducing pain behaviour in the CCI model. This has never been done with the use of the PSNI model.

9 rats received 5 μl Remicade (infliximab) 10 mg/ml ICV days 2, 3 and 4 postoperatively (PSNI). The control which included 10 rats received 5 μl sterile 0.9% NaCl solution (Sigma) ICV. Both groups were tested at days 2, 3, 4, 6 and 8 postoperatively. The experiment was blinded in a way so the surgeon did not know the administration of the individual rat.

FIG. 4 shows the nociceptive threshold for latency to hind paw withdrawal in a control group and in a group receiving infliximab 5 μl (10 mg/ml) icv at days 2, 3 and 4. Data are expressed as the difference score in percent between the basic level and the postoperative measurement. Each point represents mean±SEM, and the number of rats in each group is indicated in the parenthesis.
day 2 (infliximab 41.3±4.4%: control 41.1±7.8, P>0.05);
day 3 (infliximab 39.2±5.6%: control 37.7±4.1%, P>0.05);
day 4 (infliximab 43.4±3.8%: control 40.2±3.9%, P>0.05);
day 6 (infliximab 47.2±4.0%: control 40.1±4.1% P>0.05);
day 8 (infliximab 30.6±6.1%: control 34.0±5.4% P>0.05) (t-test).

There is no significant difference in nociceptive threshold between the infliximab administrated group compared to the control group.

There is no statistically significant difference (P>0.05) within the same group both in the control and infliximab groups at the different days measured. (ANOVA)

### Example 5

Effect of Pre-Surgical ICV Infusion of Anti-TNF-α with Osmotic Pump on Reducing Pain in the PSNI Model

8 rats received Remicade (infliximab) 10 mg/ml and the control group of 8 rats received sterile 0.9% NaCl solution (Sigma) delivered with osmotic pumps ICV. The osmotic pumps were inserted surgically day 3 prior to the PSNL operation. The animals were then tested postoperatively at days 2, 4, 6, 8 and 9.

FIG. 5 shows the nociceptive threshold for latency to hind paw withdrawal in a control group and in a group receiving infliximab (10 mg/ml) icv continuously 0.5 μl/h started day 3 preoperatively. Data are expressed as the difference score in percent between the basic level and the postoperative measurement. Each point represents mean±SEM and the number of rats in each group is indicated in the parenthesis. A significant difference between the two groups (P<0.05, Student's t-test) is marked with “*”.
day 2 (infliximab 34.2±5.4%: control 49.2±2.8, P<0.05);
day 4 (infliximab 34.6±5.4%: control 53.1±3.9%, P<0.05);
day 6 (infliximab 30.3±8.2%: control 44.5±5.6%, P>0.05);
day 8 (infliximab 27.5±5.2%: control 43.5±6.7% P>0.05). (t-test)

There is no statistically significant difference (P>0.05) within the same group, both in the control and infliximab groups at the different days (ANOVA).

### Example 6

Effect of Pre-Surgical Oral Administration of Clonidine on Neuropathic Pain in PSNI Model

This Experiments 6 and 7 are conducted in order to clarify whether p.o administration of the α2-receptor agonist clonidine started pre or post PSNI displays an effect in reducing neuropathic pain behaviours in rats when administrated subcutaneously.

9 rats (250-300 g) received clonidine (0.6 mg/kg) orally starting 2 hours pre PSNI operation, and the same dose was administrated again 6 hours after the operation. Day 1 after PSNI, the rats received 0.6 mg/kg twice. Day 2 post PSNI, the rats received 0.6 mg/kg once. The rats were tested at day 2 after operation. The control group consisting of 9 rats (250-300 g) only received water.

0.2 mg/ml clonidine solution with ordinary water as solvent was used in this experiment.

FIG. 6. shows the nociceptive threshold tested at day 2 in a group receiving clonidine started prior to PSNI compared to a control group. Data are expressed as the difference score in percent between the basic level and the postoperative measurement. Each point represents mean±SEM, and the number of determinations are indicated in parenthesis. Significant difference between the two groups P<0.05 (Student's t-test) is marked with *. (day 2: clonidine 2.6±3.1%: control 36.4±3.8%, P<0.001).

### Example 7

Effect of Post-Surgical Oral Administration of Clonidine on Neuropathic Pain in PSNI Model

6 control animals from experiment 6 received clonidine (0.6 mg/kg) once at day 4 postoperatively after the thermal hyperalgesia testing. The rats were in addition tested at days 4 and 5 after operation.

A paired t-test was used to validate the different treatments within the same group.

FIG. 7. shows the nociceptive threshold tested at days 2, 4 and 5 in a group receiving clonidine 0.6 mg/kg at day 4 postoperatively. Data are expressed as the difference score in percent between the basic level and the postoperative measurement. Each point represents mean±SEM, and the number determinations are indicated in parenthesis. Significant difference (P<0.05 in Student's t-test) is marked with *.

Comparison between pre and post administration of clonidine (day4 versus day5).

Day 4 (36.7±5.0%), day 5 (−0.2±8.5%) P<0.05; paired t-test.

There is a statistically significant difference (P<0.05) within the same group at the different measured days. Day 2 (40.3±3.8%) versus day 5 P<0.001. Day 2 versus day 4 no significance. Day 4 versus day 5, P<0.002 (ANOVA).

### Example 8

Active Vaccination with a TNF-α Variant in the PSNI Model

10 rats (400-600 g) were vaccinated according to the vaccination procedure mentioned, and 10 rats (400-600 g) were used as control group. The rats were tested at days 2, 4, 6, 8, 10, 12, 14, 19 and 25 after PSNI.

FIG. 8 shows the nociceptive threshold expressed as latency to hind paw withdrawal in a group receiving the TNF-α vaccine described above compared to a control group. Data are expressed as the difference score in percent between the basic level and the postoperative measurement. Each point represents mean±SEM, and the number of rats is indicated in the parenthesis.

No significant differences in difference score (P>0.05, t-test) were found between the two groups at any of the measured days and there is no statistically significant difference (P>0.05) in the mean among the same group both in the control and infliximab groups at the different days measured (ANOVA).

### Example 9

Active Vaccination with a TFN-α Variant in the CCI Model

10 rats (400-600 g) were vaccinated according to the vaccination procedure mentioned, and 10 rats (400-600 g) were used as control group. The rats were tested for thermal hyperalgesia at days 3, 4, 6, 8, 10, 14, 19 and 26 post CCI.

FIG. 9 shows the nociceptive threshold expressed as latency to hind paw withdrawal in a group receiving the TNF-α vaccine compared to a control group. Data are expressed as the difference score in percent between the operated left leg and the unoperated right leg. Each point represents mean±SEM, and the number of rats is indicated in the parenthesis. Significant differences (Student's t-test p<0.05) are indicated with “*”.

Day 3(vaccine 32.1±5.9%: control 41.3±7.2%, P>0.05); day 4 (vaccine 36.9±4.5%: control 50.2±3.3%, P<0.05); day 6 (vaccine 39.0±2.5%: control 49.7±3.2%, P<0.05); day 8 (vaccine 38.7±3.0%: control 51.0±3.8%, P>0.05); day 10 (vaccine 39.3±4.8%: control 50.3±3.4%, P>0.05); day 12 (vaccine 43.1±3.5%: control 49.2±4.6%, P>0.05); day 14 (vaccine 43.3±3.7%: control 50.4±4.0%, P>0.05); day 19 (vaccine 36.6±3.0%: control 49.4±3.1%, P<0.05); day 26 (vaccine 35.9±4.2%: control 46.6±4.4%, P>0.05). (t-test).

### References

Aley K O, Reichling D B, Levine J D (1996). Vincristine hyperalgesia in the rat: a model of painful Vincristine neuropathy in humans. Neuroscience, 73: 259-65.
Ali Z, Meyer R A, Campbell J N (1996). Secondary hyperalgesia to mechanical but not heat stimuli following a capsaicin injection in hairy skin.Pain, 68: 401-411.
Amir R, Devor M (2000). Functional cross-excitation between afferent A- and C-neurons in dorsal root ganglia. Neuroscience, 95: 1: 189-195.
Attal N, Bouhassira D (1999). Mechanisms of pain in peripheral neuropathy. Acta Neurologica Scandinavia, Supplement 173: 12-24.
Baldwin R L, Stolowitz M L, Hood L, Wisnieski B J (1996). Structural changes of tumor necrosis factor α associated with membrane insertion and channel formation. Proc. Natn. Acad. Sci. U.S.A., 93: 1021-1026.
Barnes P F, Chatterjee D, Brennan P J, Rea Th, Modlin R L (1992). Tumor necrosis factor production in patients with leprosy. Infect. Immun., 60: 1441-1446.
Bennett G J (2000). Update on the neurophysiology of pain transmission and modulation: Focus on the NMDA-Receptor. Journal of Pain and Symptom Management, 19: 1 Supplement.
Bennet G J (1999). Does a neuroimmune interaction contribute to the genesis of painful peripheral neuropathies? Proc. Natl. Acad. Sci. USA, 96: 7737-7738.
Bennett G J (1998). Neuropathic pain: New insights, new inteventions. Hospital Practise, issue 10.
Bennett G J, Xie Y-K (1988). A peripheral mononeuropathy in rat that produces disorders of pain sensation like those seen in man. Pain, 33: 87-107.
Besson J M, Chaouch A (1987). Peripheral and spinal mechanisms of nociception. Physiol Rev 67: 67-186.
Besson J M (1999). The neurobiology of pain. The Lancet, 353: 1610-15.
Castro-Lopez J M, Tavares I, Coimbra A (1993). GABA decreases in the spinal cord dorsal horn after peripheral neurectomy. Brain Res, 620: 287-91.
Cervero F, Meyer R A, Campbell J N (1994). A psycophysical study of secondary hyperalgesia: evidence for increased pain due to input from nociceptors. Pain 58: 21-28.
Chaplan S R, Bach F W, Pogrel J D, Chung J M, Yaksh T L (1994). Quantitative assessment of tactile allodynia in the rat paw. Journal of Neuroscience Methods, 53: 55-63.
Chi S I, Levine J D, Basbaum A I (1993). Peripheral and central contributions to the persistent expression of spinal cord fos-like immunoreactivity produced by sciatic nerve transection in the rat. Brain Res, 617: 225-37.
Clathworthy A L, Illich P A, Castro G A, Walters E T (1995). Role of periaxonal inflammationin the development of thermal hyperalgesiaand guarding behavior in a rat model of neuropathic pain. Neuroscience letters, 184: 5-8.
Covey W C, Ignatowski T A, Knight P R, Spengler R N (2000). Brain-derived TNFα: involvement in neuroplastic changes implicated in the conscious perception of persistent pain. Brain Research, 859: 113-122.
Cui J G, Holmin S, Mathiesen T, Meyerson A B, Linderoth B (2000). Possible role of inflammatory mediators in tactile hypersensitivity in rat models of mononeuropathy. Pain, 88: 3: 239-48
Cunha F Q, Poole S, Lorenzetti B, Ferreira S H (1992). The pivotal role of tumor necrosis factor α in the development of inflammatory hyperalgesia. Br. J. Pharmac., 107: 660-664.
Devor M, Govrin-Lippmann R, Angelides K (1993). Na+ channel immunolocalization in peripheral mammalian axons and changes following nerve injury and neuroma formation. J. Neurosci, 13: 1976-1992.
Ding M, Hart R P, Jonakait G M (1995) Tumor necrosis factor-alpha induces substance P in sympathetic ganglia through sequential induction of interleukin-1 and leukemia inhibitory factor. J Neurbiol, 28: 445-454.
Dixon W R, Mosimann W F, Weiner N (1979). The role of presynaptic feedback mechanisms in regulation of norepinephrine release by nerve stimulation. J Pharmacol. Exp. Ther., 209: 196-204
Dixon W J (1980). Efficient analysis of experimental observations. Ann. Rev. Pharmacol. Toxicol., 20: 441-462.
Elenkov I J, Kovacs K, Duda E, Stark E, Vizi E S (1992). Presynaptic inhibitory effect of TNF-α on the release of noradrenaline in isolated median eminence. J Neuroimmunol, 41: 117-120.
Eliav E, Herzberg U, Ruda M A, Bennett G J (1999). Neuropathic pain from an experimental neuritis of the rat sciatic nerve. Pain, 83: 169-182.
Empl M, Renaud S, Erne B, Fuhr P, Straube A, Schaeren-Wiemers N, Steck A J (2001). TNF-alpha expression in painful and nonpainful neuropathies. Neurology, 56:1371-1377.
George A, Schmidt C, Weishaupt A, Toyka K V, Sommer C (1999). Serial determination of tumor necrosis factor-alpha content in rat sciatic nerve after chronic constriction injury. Exp Neurol, 160: 124-32.
George A, Marziniak M, Schäfers M, Toyka K V, Sommer C (2000). Thalidomide treatment in chronic constrictive neuropathy decreases endoneurial tumor necrosis factor-a, increases interleukin-10 and has long-term effects on spinal cord dorsal horn met-enkephalin. Pain, 88: 267-275.
Grant S J, Redmond D E (1981). The neuroanatomy and pharmacology of the nucleus locus coeruleus. Prog Clin Biol Res, 71: 5-27.
Hattori A, Hayashi K, Kohno M (1996). Tumor necrosis factor (TNF) stimulates the production of nerve growth factor in fibroblasts via the 55 kDa type 1 TNF receptor. FEBS Lett, 379:157-160.
Ignatowski T A, Chou R C, Spengler R N (1996) Changes in noradrenergic sensitivity to tumor necrosis factor-α in brains of rats administered clonidine. Journal of Neuroimmunology, 70: 55-63.
Ignatowski T A, Covey W C, Knight P R, Severin C M, Nickola T J, Spengler R N (1999). Brain derived TNFa mediates neuropathic pain. Brain Research, 841: 70-77.
Junger H, Sorkin L S (2000). Nociceptive and inflammatory effects of subcutaneous TNFα. Pain, 85: 145-151
Kagan B L, Baldwin R L, Munoz D, Wisnieski B J (1992). Formation of ion-permeable channels by tumor necrosis factor-α. Science, 255: 1427-1430.
Kajander K C, Bennett G J (1992). The onset of a painful peripheral neuropathy in rat: a partial and differential deafferentation and spontaneous discharge in Ab and Ad primary afferent neurons. J Neurophysiol, 68: 734-44.
Kajander K C, Wakisaka S, Bennett G J (1992b). Spontaneous discharge originates in the dorsal root ganglion at the onset of a painful peripheral neuropathy in the rat. Neuroscience Letters, 138: 225-228.
Khanna S (1997). Dorsal hippocampus field CA1 pyramidal cell responses to a persistent versus an acute nociceptive stimulus and their spatial modulation. Neuroscience, 77: 713-721.
Khanna S, Sinclair J G (1989). Noxious stimuli produces prolonged changes in the CA1 region of the rat hippocampus. Pain 39: 337-343.
Kim K J, Yoon Y W, Chung J M (1997). Comparison of three rodent neuropathic pain models. Experimental Brain Research, 113: 200-6.
Kim S H, Chung J M (1992). An experimental model for peripheral neuropathy produced by segmental spinal nerve ligation in the rat. Pain, 50: 355-363.
Laird J M, Bennett G J (1993). An electrophysiological study of dorsal horn neurons in the spinal cord of rats with an experimental neuropathy. J neurophysiol, 69: 2072-85.
Lindenlaub T, Sommer C (2000a). Partial sciatic nerve transection as a model of neuropathic pain: a qualitative and quantitative neuropathological study. Pain, 89: 97-106.
Lindenlaub T, Teuteberg P, Hartung T, Sommer C (2000b). Effects of neutralizing antibodies to TNF-alpha on pain-related behavior and nerve regeneration in mice with chronic constriction injury. Brain res, 866: 15-22.
Lindholm D, Heumann R, Meyer M, Thoenen H (1987). Interleukin-1 regulates synthesis of nerve growth factor in non-neuronal cells of rat sciatic nerve. Nature, 330: 658-659.
Lisney S J W, Devor M (1987). Afterdischarge and interactions among fibers in damaged peripheral nerve in the rat. Brain Res 415: 122-36.
Ma W, Bisby M A (1998). Partial and complete sciatic nerve injuries induce similar increases of neuropeptide Y and vasoactive intestinal peptide immunoreactivities in primary sensory neurons and their central projections. Neuroscience, 86: 1217-1234.
MacFarlane B V, Wright A, O'Callaghan J, Benson H A E (1997). Chronic neuropathic pain and its control by drugs. Pharmacol Ther, 75: 1-19.
Margalit D, Segal M (1979). A pharmacologic study of analgesia produced by stimulation of the locus coeruleus. Psychopharmacology, 62: 169-173.
Maves T, Pechman P, Gebhart G, Melter S (1993). Possible chemical contribution from chromic gut sutures produces disorders of pain sensation like those seen in man. Pain, 54: 57-69.
McLachlan E M, Jänig W, Devor M, Michaelis M (1993). Peripheral nerve injury triggers noradrenergic sprouting within dorsal root ganglia. Nature 363: 543-6
Miki K, Fukuoka T, Tokunaga A, Noguchi K (1998). Calcitonin gene-related peptide increase in the rat spinal horn and dorsal column nucleus following peripheral nerve injury: up-regulation in a subpopulation of primary afferent sensory neurons. Neuroscience, 82: 1243-52.
Millan M J (1999). The induction of pain: an integrative review. Progress in Neurobiology, 57: 1-164.
Moreira A L, Sampaio E P, Zmuidzinas A, Frindt P, Smith K A, Kaplan G (1993). Thalidomide exerts its inhibitory action on tumor necrosis factor a by enhancing mRNA degradation. J Exp. Med, 177: 1675-1680.
Myers R R, Heckman H M, Galbraith J A, Powell H C (1991). Subperineurial demyelination associated with reduced nerve blood flow and oxygen tension after epineurial vascular stripping. Lab invest, 65: 41-50.
Nickola T J, Ignatowski T A, Spengler R N (2000). Antidepressant drug administration modifies the interactive relationship between α2-adrenergic sensitivity and the levels of TNF in the rat brain. Journal of Neuroimmunology, 107: 50-58.
Nickola T J, Ignatowski T A, Reynolds J L, Spengler R N (2001). Antidepressant drug-induced alterations in neuron-localized tumor necrosis factor-α mRNA and a2-adrenergic sensitivity. The Journal of Pharmacology and Experimental Therapeutics, 297: 680-687.
Nicol G D, Lopshire J C, Pafford C M (1997) Tumor necrosis factor enhances the capsaicin sensitivity of rat sensory neurons. J Neurosci, 17: 975-982.
Nicholson B (2000). Gabapentin use in neuropathic pain syndromes. Acta Neurologica Scandinavia, 101: 359-371.
Perry V H, Brown M C, Andersson P B (1993). Machrophage responses to central and peripheral nerve injury. Adv Neurol, 59: 309-314.
Pitcher G M, Ritchie J, Henry J L (1999). Nerve constriction in the rat: model of neuropathic, surgical and central pain. Pain, 83: 1: 37-46
Prado W A, Roberts M H T (1985). An assessment of the antinociceptive and aversive effects of stimulating identified sites in the rat brain. Brain Research, 340: 219-228.
Proudfit H K (1998). Pharmacological evidence for the modulation of nociception by noradrenergic neurons. Prog Brain Res, 77: 357-370.
Redford E J, Hall S M, Smith K J (1995). Vascular changes and demyelination induced by intraneural injection of tumor necosis factor. Brain, 118: 869-878.
Reinhard J F, Roth R H (1982). Noradrenergic modulation of serotonin synthesis and metabolism. Inhibition by clondine in vivo. J Pharmacol Exp Ther, 221: 541-546.
Sampaio E P, Moreira A L, Sarno E N, Malta A M, Kaplan G (1991). Thalidomide selectively inhibits tumor necrosis factor α production by stimulated human monocytes. J Exp Med, 173: 699.
Scadding J W (1981). Development of ongoing activity, mechanosensitivity, and adrenalin sensitivity in severed peripheral nerve axons. Exp Neurol, 73: 345-64.
Schmalbruch H (1986). Fiber composition of the rat sciatic nerve. The anatomical record, 215: 71-81.
Seltzer Z, Dubner R, Shir Y (1990). A novel behavioral model of neuropathic pain disorders produced in rats by partial sciatic nerve injury. Pain, 43: 228-240.
Shir Y, Seltzer Z (1991). Effects of sympathectomy in a model of causalgiform pain produced by partial sciatic nerve injury in rats. Pain, 45: 309-20.
Sindrup S H, Jensen T S (1999). Efficacy of pharmacological treatments of neuropathic pain: an update and effect related to mechanisms of drug action. Pain, 83: 389-400.
Soliven B, Albert J (1992). Tumor necrosis factor modulates the inactivation of catecholamine secretion in cultured sympathetic neurons. J Neurochem, 58: 1073-1078.
Sommer C, Galbraith J A, Heckman H M, Myers R R (1993). Pathology of experimental compression neuropathy producing hyperestesia. J Neuropathol Exp Neurol; 52: 223-233.
Sommer C, Marziniak M, Myers R R (1998a) The effect of thalidomide treatment on vascular pathology and hyperalgesia caused by chronic constriction injury of rat nerve. Pain, 74: 83-91
Sommer C, Schmidt C, George A (1998b). Hyperalgesia in experimental neuropathy is dependent on the TNF Receptor 1. Experimental Neurology, 151: 138-142.
Sommer C (2001). Zytokine bei neuropatischen Schmerzen. Der Anaesthesist, 50: 416-426.
Sommer C, Lindenlaub T, Teuteberg P, Schäfers M, Hartung T, Toyka K V (2001a). Anti-TNF-neutralizing antibodies reduce pain-related behavior in two different mouse modelss of painful mononeuropathy. Brain Research, 913: 86-89.
Sommer C, Schäfers M, Marziniak M, Toyka K V (2001b). Etanercept reduces hyperalgesia in experimental painful neuropathy. J Peripher Nerv Syst, 6: 67-72.
Sorkin L S, Xiao W H, Wagner R, Myers R R (1997). Tumor necrosis factor-α induces ectopic activity in nociceptive primary afferent fibres. Neuroscience, 81: 255-262.
Swanson L W, Hartman B K (1975). The central adrenergic system. An immunofluorescence study of the location of cell bodies and their efferent connections in the rat utilizing dopamine-beta-hydroxylase as a marker. J Comp Neurol, 163: 467-505.
Treede R D, Meyer R A, Raja S N, Campbell J N (1992). Peripheral and central mechanisms of cutaneous hyperalgesia. Progress in neurobiology, 38: 397-421.
Vogel C, Lindenlaub T, Tiegs G, Toyka K V, Sommer C (2000). Pain related behavior in TNF-receptor deficient mice. Progress in Pain Research and Management, 16: 249-257.
Wagner R, Myers R R (1996). Schwann cells produce tumor necrosis factor alpha: expression in injured and non-injured nerves. Neuroscience, 73: 625-629.
Wagner R, Janjigian M, Myers R R (1998). Anti-inflammatory interleukin-10 therapy in CCI neuropathy decreases thermal hyperalgesia, macrophage recruitment and endoneurial TNF-α lpha expression. Pain, 74: 35-42.
Walker K, Fox A J, Urban L A (1999). Animal models for pain research. Molecular medicine today, July: 5: 319-321.
Wall P D, Gutnick M (1974). Properties of afferent nerve impulses originating from a neuroma. Nature, 248: 740-743.
Wall P D, Gutnick M (1975). Ongoing activity in peripheral nerves: the physiology and pharmacology of impulses originating from a neuroma. Exp Neurol, 43: 580-93
Wall P D, Devor M (1981). The effect of peripheral nerve injury on dorsal root potentials and on transmission of afferent signals to the spinal cord. Brain Res, 209: 95-111.
Waxman S G, Cummins T R, Dib-Hajj S D, Black J A (2000). Voltage-gated sodium channels and the molecular pathogenesis of pain: a review. Journal of Rehabilitation Research and Development, 37: September/October 5.
Woolf C J, Wall P D (1992). Chronic peripheral nerve section diminishesthe primary A-fibre mediated inhibition of rat dorsal horn neurons. Brain Res, 242: 77-85.
Woolf C J, Allchorne A, Safieh-Garabedian B, Poole S (1997). Cytokines, nerve growth factor and inflammatory hyperalgesia: the contribution of tumor necrosis factor alpha. Br J Pharmacol, 121: 417-424.
Woolf C J, Mannion R J (1999). Neuropathic pain: aetiology, symptoms,mechanisms, and management. The Lancet, 353: June 5: 1959-64.
Wuarin-Bierman L, Zahnd G R, Kaufman F, Burcklen L, Adler J (1987). Hyperalgesia in spontaneous and experimental models of diabetic neuropathy. Diabetologia, 30: 653-8.
Yaksh T L (1985). Pharmacology of spinal adrenergic systems which modulate spinal nociceptive processing. Pharmacol Biochem Behav, 22: 845-858.
Yamamoto T, Nozaki-Taguchi N (1996). Clonidine but not morphine delays the development of thermal hyperesthesia induced by sciatic nerve constriction injury in the rat. Anesthesiology, 85: 835-845.
Zhang C E, Yang S W, Guo Y G, Qiao J T, Dafny N (1997). Locus coeruleus stimulation modulates the nociceptive response in parafascicular neurons: an analysis of descending and ascending pathways. Brain Res Bull, 42: 273-278.
Ziegler E A, Magerl W, Meyer R A, Treede R D (1999). Secondary hyperalgesia to punctate mechanical stimuli. Central sensitization to A-fibre nociceptor input. Brain, 122: 2245-57.

The invention will now be further described by the following numbered paragraphs:

1. A method for reducing pain or increasing the threshold for nociception in an individual in need thereof, the method comprising administering an effective amount of an agent capable of inducing an active immune response that targets said indivual's autologous tumour necrosis a (TNFα).

2. The method according to paragraph 1, wherein the agent is selected from the group consisting of an immunogenic TNFα variant, a nucleic acid fragment encoding an immunogenic TNFα variant, and a non-pathogenic bacterium or virus that harbours a nucleic acid fragment encoding an immunogenic TNFα variant.

3. The method according to paragraph 1 or 2, wherein the pain is neuropathic pain.

4. The method according to any of the preceding paragraphs, wherein the agent is an immunogenic TNFα variant selected from the group consisting of
a substantially complete TNFα monomer, dimer or trimer that is conjugated to an immunogenic carrier molecule,
a fragment of a TNFα monomer, that is conjugated to an immunogenic carrier molecule,
a TNFα monomer, dimer or trimer wherein is introduced at least one foreign T_{H} epitope by means of insertion addition or substitution,
a monomer that mimics a TNFα multimer structure and that includes at least one foreign T_{H} epitope, and
a chimeric carrier construct that comprises an inert carrier moiety to which is coupled at least one B-cell epitope of TNFα and at least one foreign T_{H} epitope.

5. The method according to any of the preceding paragraphs, wherein the TNFα is human TNFα.

6. The method according any one of the preceding paragraphs, wherein immunogenic TNFα variant is selected from the group consisting of
two or three complete TNFα monomers joined end-to-end by a peptide linker, wherein at least one peptide linker includes at least one MHC Class II binding amino acid sequence,
two or three complete TNFα monomers joined end-to-end by an inert peptide linker, wherein at least one of the monomers include at least one foreign MHC Class II binding amino acid sequence or wherein at least one foreign MHC Class II binding amino acid sequence is fused to the N- or C-terminal monomer, optionally via an inert linker.

7. The method according to any one of paragraphs 1-5, wherein immunogenic TNFα variant includes at least one foreign MHC Class II binding amino acid sequence and further has the characteristic of being
a human TNFα monomer or an immunogenic TNFα variant as defined in any one of paragraphs 4-6, wherein has been inserted or in-substituted at least one foreign MHC Class II binding amino acid sequence into flexible loop 3, and/or
a human TNFα or an immunogenic TNFα variant as defined in any one of paragraphs 4-6, wherein has been introduced at least one disulfide bridge that stabilises the TNFα monomer 3D structure, and/or
a human TNFα monomer or an immunogenic TNFα variant as defined in any one of paragraphs 4-6, wherein any one of amino acids 1, 2, 3, 4, 5, 6, 7, 8, and 9 in the amino terminus have been deleted, and/or
a human TNFα monomer or an immunogenic TNFα variant as defined in any one of paragraphs 4-6, wherein an inserted or in-substituted at least one foreign MHC Class II binding amino acid sequence into loop 1 in an intron position, and/or
a human TNFα monomer or an immunogenic TNFα variant as defined in any one of paragraphs 4-6, wherein at least one foreign MHC Class II binding amino acid sequence is introduced as part of an artificial stalk region in the N-terminus of human TNFα, and/or
a human TNFα monomer or an immunogenic TNFα variant as defined in any one of paragraphs 4-6, wherein at least one foreign MHC Class II binding amino acid sequence is introduced so as to stabilize the monomer structure by increasing the hydrophobicity of the trimeric interaction interface, and/or
a human TNFα monomer or an immunogenic TNFα variant as defined in any one of paragraphs 4-6, wherein at least one foreign MHC Class II binding amino acid sequence flanked by glycine residues is inserted or in-substituted in the TNFα amino acid sequence, and/or
a human TNFα monomer or an immunogenic TNFα variant as defined in any one of paragraphs 4-6, wherein at least one foreign MHC Class II binding amino acid sequence is inserted or in-substituted in the D-E loop, and/or
a human TNFα monomer or an immunogenic TNFα variant as defined in any one of paragraphs 4-6, wherein at least one foreign MHC Class II binding amino acid sequence is inserted or in-substituted between two identical subsequences of human TNFα, and/or
a human TNFα monomer or an immunogenic TNFα variant as defined in any one of paragraphs 4-6, wherein at least one salt bridge in human TNFα has been strengthened or substituted with a disulphide bridge, and/or
a human TNFα monomer or an immunogenic TNFα variant as defined in any one of paragraphs 4-6, wherein solubility and/or stability towards proteolysis is enhanced by introducing mutations that mimic murine TNFα crystalline structure, and/or
a human TNFα monomer or an immunogenic TNFα variant as defined in any one of paragraphs 4-6, wherein potential toxicity is reduced or abolished by introduction of at least one point mutation.

8. The method according to any one of the preceding paragraphs, wherein the amino acid sequence of the immunogenic TNFα variant is selected from the group consisting of SEQ ID NO: 18, 23, 24, 25, 26, 27, 28, 29, 30, 31, 32, 33, 34, 35, 36, 37, 38, 39, 40, 41, 42, 43, 44, 45, 46, 47, 49, 51, 53, 55, 57, and 59 from the sequence listing of PCT/DK02/00764, and any amino acid sequence that only include conservative amino acid changes thereof.

1. A method for reducing pain or increasing the threshold for nociception in an individual in need thereof, the method comprising administering an effective amount of an agent capable of inducing an active immune response that targets said indivual's autologous tumour necrosis a (TNFα).

2. The method according to claim 1, wherein the agent is selected from the group consisting of an immunogenic TNFα variant, a nucleic acid fragment encoding an immunogenic TNFα variant, and a non-pathogenic bacterium or virus that harbours a nucleic acid fragment encoding an immunogenic TNFα variant.

3. The method according to claim 1, wherein the pain is neuropathic pain.

4. The method according to claim 1, wherein the agent is an immunogenic TNFα variant selected from the group consisting of
a substantially complete TNFα monomer, dimer or trimer that is conjugated to an immunogenic carrier molecule,
a fragment of a TNFα monomer, that is conjugated to an immunogenic carrier molecule,
a TNFα monomer, dimer or trimer wherein is introduced at least one foreign T_{H} epitope by means of insertion addition or substitution,
a monomer that mimics a TNFα multimer structure and that includes at least one foreign T_{H} epitope, and
a chimeric carrier construct that comprises an inert carrier moiety to which is coupled at least one B-cell epitope of TNFα and at least one foreign T_{H} epitope.

5. The method according to claim 1, wherein the TNFα is human TNFα.

6. The method according to claim 1, wherein immunogenic TNFα variant is selected from the group consisting of
two or three complete TNFα monomers joined end-to-end by a peptide linker, wherein at least one peptide linker includes at least one MHC Class II binding amino acid sequence; and,
two or three complete TNFα monomers joined end-to-end by an inert peptide linker, wherein at least one of the monomers include at least one foreign MHC Class II binding amino acid sequence or wherein at least one foreign MHC Class II binding amino acid sequence is fused to the N- or C-terminal monomer, optionally via an inert linker.

7. The method according to claim 1, wherein immunogenic TNFα variant includes at least one foreign MHC Class II binding amino acid sequence and further has the characteristic of being a human TNFα monomer or an immunogenic TNFα variant selected from the group consisting of
a substantially complete TNFα monomer, dimer or trimer that is conjugated to an immunogenic carrier molecule,
a fragment of a TNFα monomer, that is conjugated to an immunogenic carrier molecule,
a TNFα monomer, dimer or trimer wherein is introduced at least one foreign T_{H} epitope by means of insertion addition or substitution,
a monomer that mimics a TNFα multimer structure and that includes at least one foreign T_{H} epitope, and
a chimeric carrier construct that comprises an inert carrier moiety to which is coupled at least one B-cell epitope of TNFα and at least one foreign T_{H} epitope;
wherein
at least one foreign MHC Class II binding amino acid sequence has been inserted or in-substituted into flexible loop 3, and/or
at least one disulfide bridge that stabilises the TNFα monomer 3D structure has been introduced, and/or
any one of amino acids 1, 2, 3, 4, 5, 6, 7, 8, and 9 in the amino terminus have been deleted, and/or
at least one foreign MHC Class II binding amino acid sequence has been inserted or in-substituted into loop 1 in an intron position, and/or
at least one foreign MHC Class II binding amino acid sequence is introduced as part of an artificial stalk region in the N-terminus of human TNFα, and/or
at least one foreign MHC Class II binding amino acid sequence is introduced so as to stabilize the monomer structure by increasing the hydrophobicity of the trimeric interaction interface, and/or
at least one foreign MHC Class II binding amino acid sequence flanked by glycine residues is inserted or in-substituted in the TNFα amino acid sequence, and/or
at least one foreign MHC Class II binding amino acid sequence is inserted or in-substituted in the D-E loop, and/or
at least one foreign MHC Class II binding amino acid sequence is inserted or in-substituted between two identical subsequences of human TNFα, and/or
at least one salt bridge in human TNFα has been strengthened or substituted with a disulphide bridge, and/or
solubility and/or stability towards proteolysis is enhanced by introducing mutations that mimic murine TNFα crystalline structure, and/or
potential toxicity is reduced or abolished by introduction of at least one point mutation.

8. The method according to claim 1, wherein the amino acid sequence of the immunogenic TNFα variant is selected from the group consisting of SEQ ID NO: 18, 23, 24, 25, 26, 27, 28, 29, 30, 31, 32, 33, 34, 35, 36, 37, 38, 39, 40, 41, 42, 43, 44, 45, 46, 47, 49, 51, 53, 55, 57, and 59 from the sequence listing of PCT/DK02/00764, and any amino acid sequence that only includes conservative amino acid changes thereof.
